(19) United States
(12) Patent Application Publication
(10) Pub. No.: US 2025/0368611 A1
(43) Pub. Date: Dec. 4, 2025

(54) METHOD FOR PREPARING BERAPROST 314-D SODIUM

(71) Applicants: YS Life Science Co., Ltd., Hwaseong-si (KR); CytoAgents, Inc., Pittsburgh, PA (US)

(72) Inventors: Chang-Young OH, Hwaseong-si (KR); Kee-Young LEE, Hwaseong-si (KR); Seung Jong LEE, Hwaseong-si (KR); Ganganna BOGONDA, Hwaseong-si (KR); Areum BAEK, Hwaseong-si (KR); Eunim JEONG, Hwaseong-si (KR); Jodi CRAIGO, Cranberry Township, PA (US); Todd J. LORENZ, Orinda, CA (US); Arthur P. BERTOLINO, Holland, MI (US); Teresa WHALEN, Pittsburgh, PA (US); Heather Lynn NOTTINGHAM, Carmel Valley, CA (US)

(73) Assignees: YS Life Science Co., Ltd., Hwaseong-si (KR); CytoAgents, Inc., Pittsburgh, PA (US)

(21) Appl. No.: 19/222,873

(22) Filed: May 29, 2025

Related U.S. Application Data
(60) Provisional application No. 63/653,539, filed on May 30, 2024.

(51) Int. Cl. C07D 307/93; A61K 31/343; A61P 29/00; A61P 37/06; C07C 67/30; C07C 67/317; C07C 67/32; C07C 68/02; C07C 69/734

(52) U.S. Cl. CPC C07D 307/93; A61K 31/343; A61P 29/00; A61P 37/06; C07C 67/30; C07C 67/317; C07C 67/32; C07C 68/02; C07C 69/734; C07C 2601/10

(57) ABSTRACT

The present disclosure provides methods of synthesizing a compound of Formula I. The method proceeds through several different pathways including a radical cyclization. Also disclosed are compositions the compound of Formula I as well as methods of using the compound of Formula I in the treatment several conditions or disorders.

### CROSS-REFERENCE TO RELATED APPLICATIONS

This application claims priority to U.S. Provisional Application No. 63/653,539, which was filed on May 30, 2024, and is hereby incorporated by reference herein in its entirety.

### SUMMARY

The present disclosure includes embodiments directed to methods of preparing a compound of Formula I having the structure:

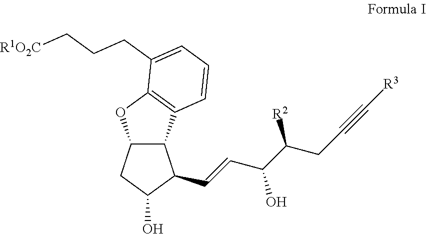

The present disclosure also relates to formulations comprising Formula I that has been prepared by the methods disclosed herein.

### BACKGROUND

Beraprost 314-d sodium (esuberaprost sodium, BPS-314d, CTO1681, or sodium 4-((1R,2R,3aS,8bS)-2-hydroxy-1-((3S,4S,E)-3-hydroxy-4-methyloct-1-en-6-yn-1-yl)-2,3,3a,8b-tetrahydro-1H-cyclopenta[b]benzofuran-5-yl)butanoate), is a useful compound for the prevention and treatment of cytokine release syndrome (CRS) for CAR-T therapy.

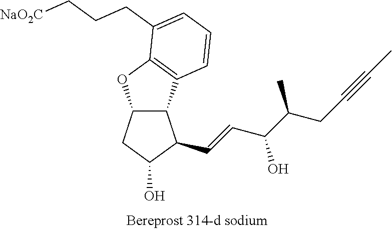

To synthesize a biologically active isomer of beraprost, such as beraprost 314-d, conventional synthetic procedures need resolution process of one or more intermediates. Current pharmaceutical formulations of beraprost or a similar benzoprostacyclin analogue may contain several isomers of the active pharmaceutical ingredient, of which only one is accountable for the desired pharmacologic activity. Isolation of isomers of beraprost compounds using existing synthetic methods necessitates numerous preparative HPLC or chromatographic purification operations or multiple recrystallizations, none of which are desirable or commercially practical. Enantiomeric separation can prove difficult as chiral columns may degrade over time thus reducing the efficiency of the enantiomeric separation. The loss of efficiency in the enantiomeric separation results in material containing isomers other than beraprost 314-d. Further, uniformity of formulations is particularly important for low-dose formulations, but challenging to obtain. Therefore, it is sought to develop a commercially viable, efficient synthetic approach for the single isomer of beraprost 314-d sodium with sufficient formulation uniformity for all dose-ranges, particularly low-dose formulations.

One object of the present invention is to provide a suitable methodology for high yielding construction of the tricyclic intermediate which is highly diastereomerically pure.

Another object of the present invention is to prove a cost-effective process for preparing beraprost 314-d sodium with high yields.

Another object of the present invention is to provide an effective and enantioselective synthesis of beraprost 314-d in a substantially pure form.

Another object of the present invention is to provide impurity control to synthesize a highly pure active pharmaceutical ingredient.

Another object of the present invention is to provide fewer steps than the prior synthesis.

The above outlined objectives are accomplished via the embodiments disclosed herein.

### BRIEF DESCRIPTION OF THE FIGURES

FIG. 1 depicts the UPLC chart of beraprost 314-d sodium, achiral UPLC method.

FIG. 2 depicts the HPLC chart of beraprost 314-d sodium, chiral HPLC method.

FIG. 3A shows that CTO1681 does not interfere with the tumor-killing efficacy of CD19-targeting CAR T-cells in vitro (see line connected by square points) and dose-dependently lowers the levels of IL-6, a pro-inflammatory and CRS-inducing cytokine (see bar graph) when CAR T-cells and Raji cells are present at a 10:1 ratio.

FIG. 3B shows that CTO1681 does not interfere with the tumor-killing efficacy of CD19-targeting CAR T-cells in vitro (see line connected by square points) and dose-dependently lowers the levels of TNF-α, a pro-inflammatory and CRS-inducing cytokine (see bar graph) when CAR T-cells and Raji cells are present at a 10:1 ratio.

FIG. 3C shows that CTO1681 does not interfere with the tumor-killing efficacy of CD19-targeting CAR T-cells in vitro (see line connected by square points) and dose-dependently lowers the levels of IFN-7, a pro-inflammatory and CRS-inducing cytokine (see bar graph) when CAR T-cells and Raji cells are present at a 10:1 ratio.

FIG. 3D shows IL-6 reduction upon treatment with increasing concentrations of CTO1681, across three different concentrations of CAR-T cells to Raji cells.

FIG. 3E shows TNF-α reduction upon treatment with increasing concentrations of CTO1681, across three different concentrations of CAR-T cells to Raji cells.

FIG. 3F shows IFN-7 reduction upon treatment with increasing concentrations of CTO1681, across three different concentrations of CAR-T cells to Raji cells.

FIG. 3G shows the absence of an effect of CTO1681 on CAR-T tumor killing efficacy.

FIG. 4 shows that CTO1681 does not interfere with CAR T-cell efficacy in the reduction of lymphoma tumor burden in vivo.

### DEFINITIONS

Before the present compositions and methods are described, it is to be understood that this invention is not limited to the particular processes, formulations, compositions, or methodologies described, as these may vary. It is also to be understood that the terminology used in the description is for the purpose of describing the particular versions or embodiments only, and is not intended to limit the scope of embodiments herein which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of embodiments herein, the preferred methods, devices, and materials are now described. All publications mentioned herein are incorporated by reference in their entirety. Nothing herein is to be construed as an admission that embodiments herein are not entitled to antedate such disclosure by virtue of prior invention.

It must also be noted that as used herein and in the appended claims, the singular forms “a,” “an,” and “the” include plural reference unless the context clearly dictates otherwise.

The transitional term “comprising,” which is synonymous with “including,” “containing,” or “characterized by,” is inclusive or open-ended and does not exclude additional, unrecited elements or method steps.

In embodiments or claims where the term “comprising” is used as the transition phrase, such embodiments can also be envisioned with replacement of the term “comprising” with the terms “consisting of” or “consisting essentially of.”

As used herein, the term “consists of” or “consisting of” means that the composition, formulation or the method includes only the elements, steps, or ingredients specifically recited in the particular claimed embodiment or claim.

As used herein, the term “consisting essentially of” or “consists essentially of” means that the composition, formulation or the method includes only the elements, steps or ingredients specifically recited in the particular claimed embodiment or claim and may optionally include additional elements, steps or ingredients that do not materially affect the basic and novel characteristics of the particular embodiment or claim. For example, the only active ingredient(s) in the formulation or method that treats the specified condition (e.g., nutrient depletion) is the specifically recited therapeutic(s) in the particular embodiment or claim.

As used herein, two embodiments are “mutually exclusive” when one is defined to be something which is different from the other. For example, an embodiment wherein two groups combine to form a cycloalkyl is mutually exclusive with an embodiment in which one group is ethyl the other group is hydrogen. Similarly, an embodiment wherein one group is CH₂ is mutually exclusive with an embodiment wherein the same group is NH.

When ranges of values are disclosed, and the notation “from n1 . . . to n2” or “between n1 . . . and n2” is used, where n1 and n2 are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean plus or minus 10% of the numerical value of the number with which it is being used. Therefore, “about 50” means in the range of 45-55, “about 25,000” means 22,500 to 27,500, etc., unless the context of the disclosure indicates otherwise, or is inconsistent with such an interpretation.

The phrase “pharmaceutically acceptable salt(s)”, as used herein, includes those salts of compounds of the application that are safe and effective for use in mammals and that possess the desired biological activity. Pharmaceutically acceptable salts include salts of acidic or basic groups present in compounds of the application or in compounds identified pursuant to the methods of the application. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (that is, 1,1′-methylene-bis-(2-hydroxy-3-naphthoate)) salts. Certain compounds of the application can form pharmaceutically acceptable salts with various amino acids. Suitable base salts include, but are not limited to, aluminum, calcium, lithium, magnesium, potassium, sodium, zinc, iron and diethanolamine salts. Pharmaceutically acceptable base addition salts are also formed with amines, such as organic amines. Examples of suitable amines are N,N′-dibenzylethylenediamine, chloroprocaine, choline, diethanolamine, dicyclohexylamine, ethylenediamine, N-methylglucamine, and procaine.

The term “pure” as used herein, is used interchangeably with, the term “chemically pure”, refers to the measurement of the amount of impurities, i.e. any other kind of chemical species including isomers, found in a sample as measured by any means including, but not limited to, nuclear magnetic resonance (NMR), gas chromatography/mass spectroscopy (GC/MS), or liquid chromatography/mass spectroscopy (LC/MS).

The term “substantially free” as used herein, is used interchangeably with, the term “substantially pure”, refers to a compound which is free from all other compounds within the limits of detection as measured by any means including nuclear magnetic resonance (NMR), gas chromatography/mass spectroscopy (GC/MS), or liquid chromatography/mass spectroscopy (LC/MS). In some embodiments, substantially free may be less than about 1.0%, less than about 0.5%, less than about 0.4%, less than about 0.3%, less than about 0.2%, less than about 0.15%, less than about 0.1%, less than about 0.05%, or less than about 0.01%.

As used herein the term “immediate release” refers to compositions that release the active ingredient within a short period of time.

As used herein the term “modified release” refers to compositions that does not otherwise release the active ingredient immediately, for example it may release the active ingredient at a sustained or controlled rate over an extended period of time, or may release the active ingredient after a lag time after administration, or may be used optionally in combination with an immediate release composition. Modified release includes extended release, sustained release, and delayed release. The term “extended release” or “sustained release” as used herein is a dosage form that makes a drug available over an extended period of time after administration. The term “delayed release” as used herein is a dosage form that releases a drug at a time other than immediately upon administration.

The “weight percent” disclosed herein may be weight-to-weight percent or weight-to-volume percent, depending upon the composition.

Also provided are embodiments wherein any embodiment herein may be combined with any one or more of the other embodiments, unless otherwise stated and provided the combination is not mutually exclusive.

### DETAILED DESCRIPTION

### Methods for Preparing Formula I

The present disclosure includes embodiments directed to a method of preparing a product comprising a compound of Formula I, having the structure:

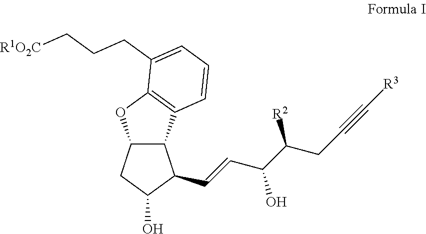

wherein
R¹ is selected from H and a cation;
R² and R³ are independently C₁₋₆ alkyl;
the method comprising the steps of:
(a) performing a condensation reaction on the compound

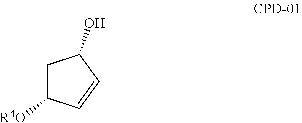

to form the compound

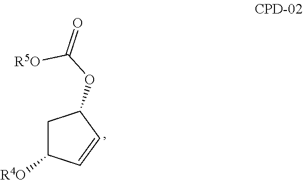

wherein R⁴ is C(O)C₁₋₆ alkyl and R⁵ is C₁₋₆ alkyl;
(b) coupling the carbonate of CPD-02 with

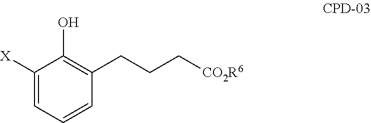

to form the compound

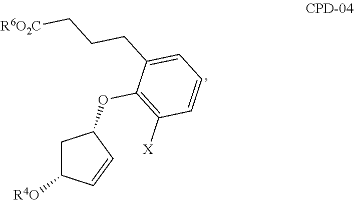

wherein is R⁶ is C₁₋₆ alkyl and X is halide;
(c) hydrolyzing CPD-04 to form the compound

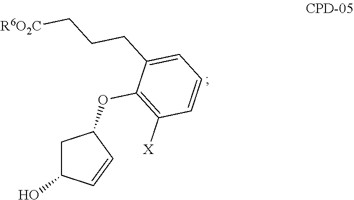

(d) protecting the alcohol of CPD-05 to form the compound

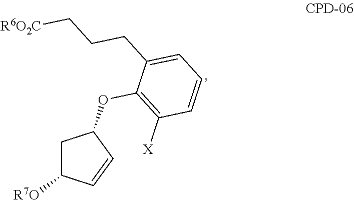

wherein R⁷ is a hydroxy protecting group;
(e) performing a radical cyclization and trapping reaction on CPD-06 with

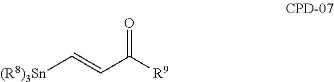

to form the compound

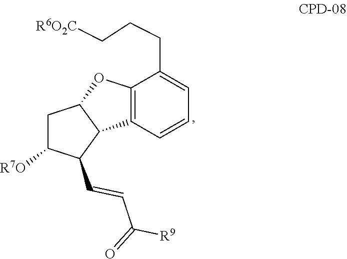

wherein R⁸ and R⁹ are independently C₁₋₆ alkyl; and
(f) converting CPD-08 to a compound of Formula I;
wherein the compound of Formula I is at least about 90% pure;
wherein the total amount of the impurities is in an amount of less than about 10.0%; and
wherein any individual impurity is present in an amount of less than about 1.0%.

In some embodiments of the method, the method further comprises cleaving the double bond of CPD-08 to from the compound

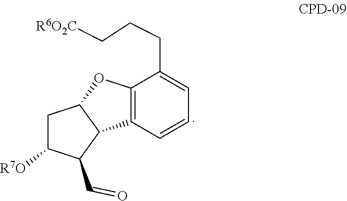

In some embodiments of the method, the method further comprises coupling CPD-09 with

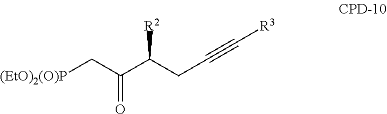

to form the compound

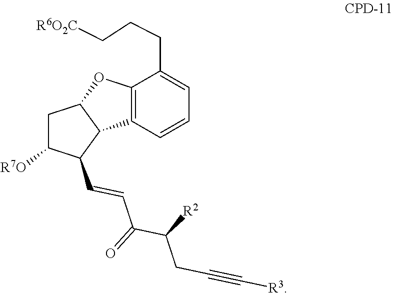

In some embodiments of the method, the method further comprises reduction of the ketone of CPD-11 to form the compound

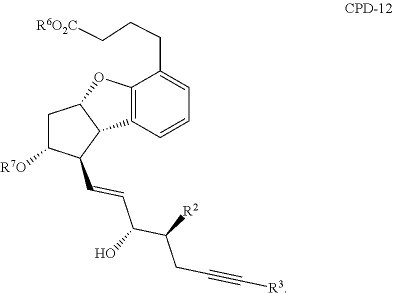

In some embodiments of the method, the method further comprises the deprotection of CPD-12 to form the compound

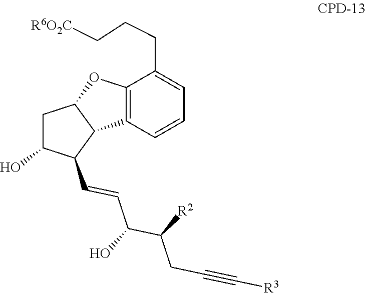

In some embodiments of the method, the method further comprises hydrolyzing CPD-13 to form the compound Formula I.

In some embodiments of the method, R¹ is Na⁺.

In some embodiments of the method, R², R³, and R⁶ are methyl.

In some embodiments of the method, R⁴ is C(O)Me.

In some embodiments of the method, R⁵ is ethyl.

In some embodiments of the method, R⁷ is tert-butyldimethylsilyl.

In some embodiments of the method, R⁸ is butyl.

In some embodiments of the method, R⁹ is pentyl.

In some embodiments of the method, the compound of Formula I is:

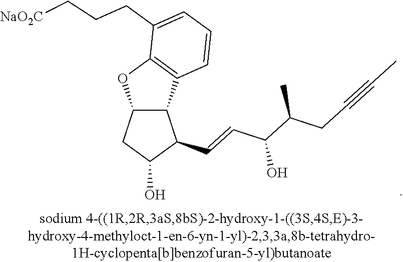

In some embodiments of the method, the method does not produce more than about 5.0% of an isomer other than the compound of Formula I, about 4.0% of an isomer other than the compound of Formula I, about 3.0% of an isomer other than the compound of Formula I, about 2.0% of an isomer other than the compound of Formula I, about 1.0% of an isomer other than the compound of Formula I, about 0.5% of an isomer other than the compound of Formula I, about 0.25% of an isomer other than the compound of Formula I, about 0.2% of an isomer other than the compound of Formula I, about 0.15% of an isomer other than the compound of Formula I, about 0.1% of an isomer other than the compound of Formula I, or about 0.05% of an isomer other than the compound of Formula I.

In some embodiments of the method, the method does not produce more than about 1.0% of an isomer other than the compound of Formula I.

In some embodiments of the method, the method does not produce more than about 0.15% of an isomer other than the compound of Formula I.

In some embodiments of the method, the method does not produce more than about 0.1% of an isomer other than the compound of Formula I.

In some embodiments of the method, the method does not produce more than about 0.05% of an isomer other than the compound of Formula I.

In some embodiments of the method, the compound of Formula I is at least about 91% pure, at least about 92% pure, at least about 93% pure, at least about 94% pure, at least about 95% pure, at least about 96% pure, at least about 97% pure, at least about 98% pure, at least about 99% pure, at least about 99.1% pure, at least about 99.2% pure, at least about 99.3% pure, at least about 99.4% pure, at least about 99.5% pure, at least about 99.6% pure, at least about 99.6% pure, at least about 99.7% pure, at least about 99.8% pure, or at least about 99.9% pure.

In some embodiments of the method, the compound of Formula I is at least about 95% pure.

In some embodiments of the method, the compound of Formula I is at least about 99% pure.

In some embodiments of the method, the compound of Formula I is at least about 99.8% pure.

In some embodiments of the method, any individual impurity is present in an amount of less than about 0.95%, less than about 0.9%, less than about 0.85%, less than about 0.8%, less than about 0.75%, less than about 0.7%, less than about 0.65%, less than about 0.6%, less than about 0.55%, less than about 0.5%, less than about 0.45%, less than about 0.4%, less than about 0.35%, less than about 0.3%, less than about 0.25%, less than about 0.2%, less than about 0.15%, less than about 0.1%, less than about 0.05%, or less than about 0.01%.

In some embodiments of the method, any individual impurity is present in an amount of less than about 0.15%.

In some embodiments of the method, any individual impurity is present in an amount of less than about 0.1%.

In some embodiments of the method, any individual impurity is present in an amount of less than about 0.05%.

In some embodiments of the method, the compound of Formula I is prepared in an overall yield of at least about 25%, at least about 24%, at least about 23%, at least about 22%, at least about 21%, at least about 20%, at least about 19%, at least about 18%, at least about 17%, at least about 16%, at least about 15%, at least about 14%, at least about 13%, at least about 12%, at least about 11%, at least about 10%, at least about 9%, at least about 8%, at least about 7%, at least about 6%, at least about 5%, at least about 4%, at least about 3%, at least about 2%, or at least about 1%.

In some embodiments of the method, the compound of Formula I is prepared in an overall yield of at least about 5%.

In some embodiments of the method, the compound of Formula I is prepared in an overall yield of at least about 10%.

In some embodiments of the method, the total amount of the impurities is in an amount of less than about 10.0%, less than about 9.0%, less than about 8.0%, less than about 7.0%, less than about 6.0%, less than about 5.0%, less than about 4.0%, less than about 3.0%, less than about 2.0%, less than about 1.0%, less than about 0.9%, less than about 0.85%, less than about 0.8%, less than about 0.75%, less than about 0.7%, less than about 0.65%, less than about 0.6%, less than about 0.55%, less than about 0.5%, less than about 0.45%, less than about 0.4%, less than about 0.35%, less than about 0.3%, less than about 0.25%, less than about 0.2%, less than about 0.15%, less than about 0.1%, less than about 0.05%, or less than about 0.01%.

In some embodiments of the method, the total amount of the impurities is in an amount of less than about 10.0%.

In some embodiments of the method, the total amount of the impurities is in an amount of less than about 5.0%.

In some embodiments of the method, the total amount of the impurities is in an amount of less than about 1.0%.

In some embodiments of the method, the total amount of the impurities is in an amount of less than about 1.0%.

In some embodiments of the method, the total amount of the impurities is in an amount of less than about 0.2%.

In some embodiments of the method, the impurity is selected from:

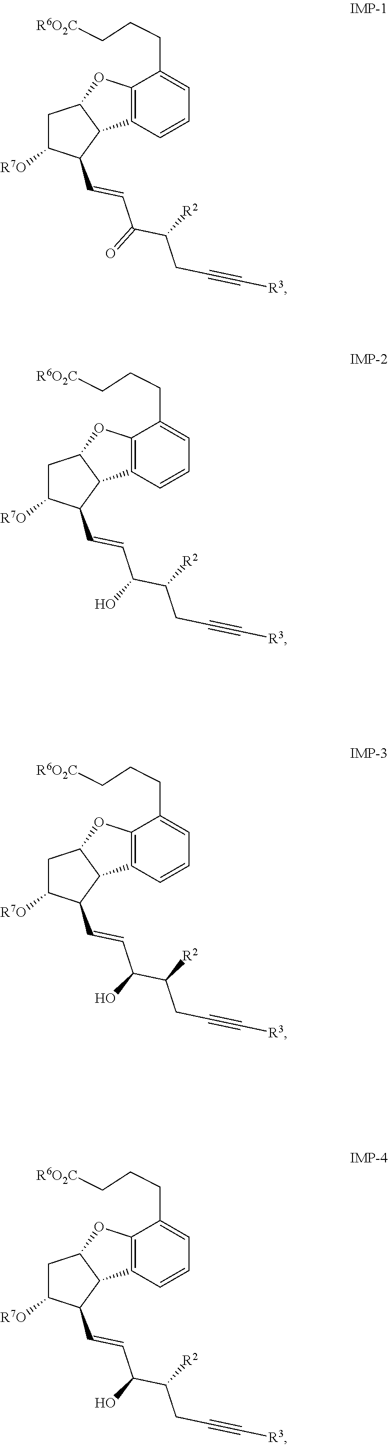

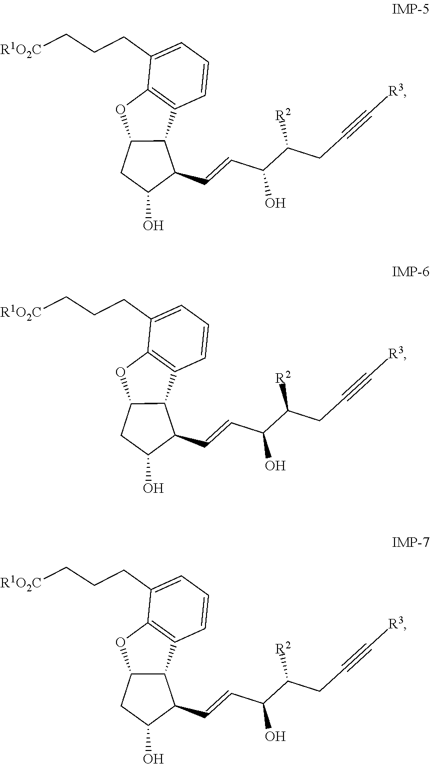

and a combination thereof.

In some embodiments of the method, the impurity is selected from:

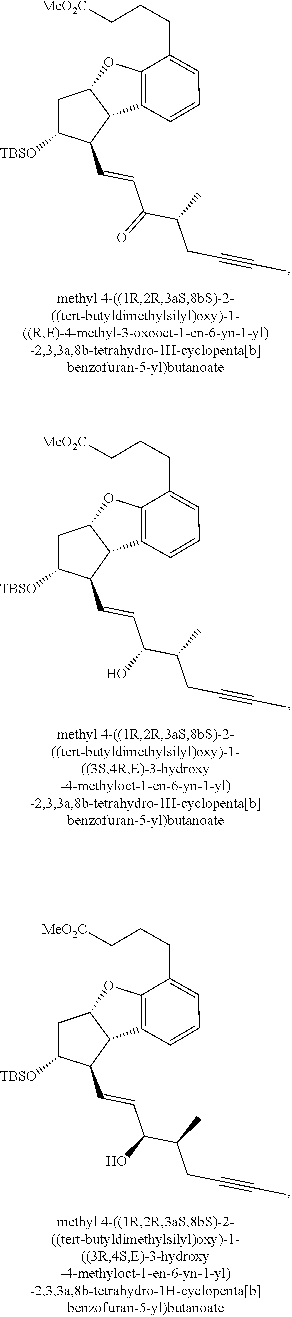 [SMILES: CC#CC[C@@H](C)C(=O)/C=C/[C@@H]1[C@H]2c3cccc(CCCC(=O)OC)c3O[C@H]2C[C@H]1O[Si](C)(C)C(C)(C)C ; CC#CC[C@@H](C)[C@H](O)/C=C/[C@@H]1[C@H]2c3cccc(CCCC(=O)OC)c3O[C@H]2C[C@H]1O[Si](C)(C)C(C)(C)C ; CC#CC[C@H](C)[C@@H](O)/C=C/[C@@H]1[C@H]2c3cccc(CCCC(=O)OC)c3O[C@H]2C[C@H]1O[Si](C)(C)C(C)(C)C]

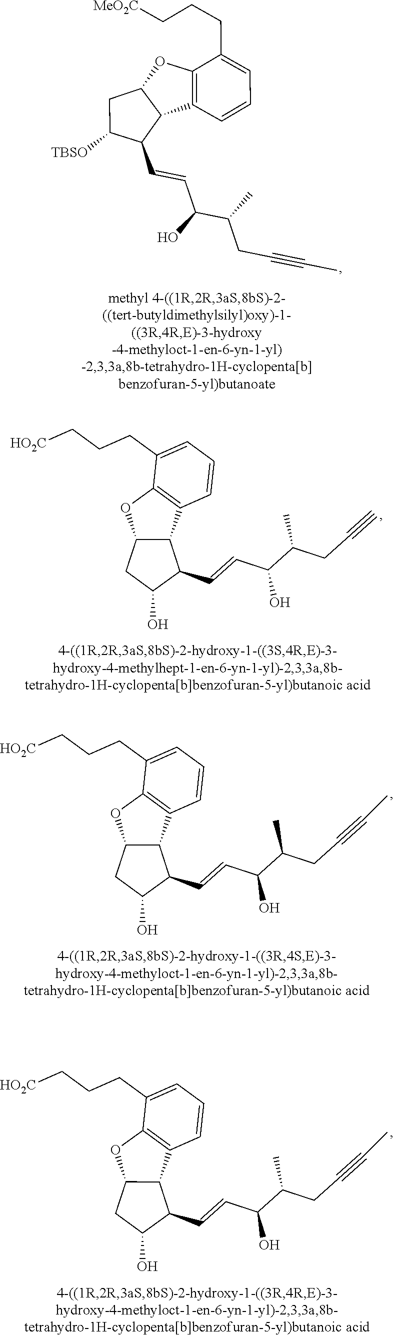 [SMILES: CC#CC[C@@H](C)[C@@H](O)/C=C/[C@@H]1[C@H]2c3cccc(CCCC(=O)OC)c3O[C@H]2C[C@H]1O[Si](C)(C)C(C)(C)C ; C#CC[C@@H](C)[C@H](O)/C=C/[C@@H]1[C@H]2c3cccc(CCCC(=O)O)c3O[C@H]2C[C@H]1O ; CC#CC[C@H](C)[C@@H](O)/C=C/[C@@H]1[C@H]2c3cccc(CCCC(=O)O)c3O[C@H]2C[C@H]1O ; CC#CC[C@@H](C)[C@@H](O)/C=C/[C@@H]1[C@H]2c3cccc(CCCC(=O)O)c3O[C@H]2C[C@H]1O]

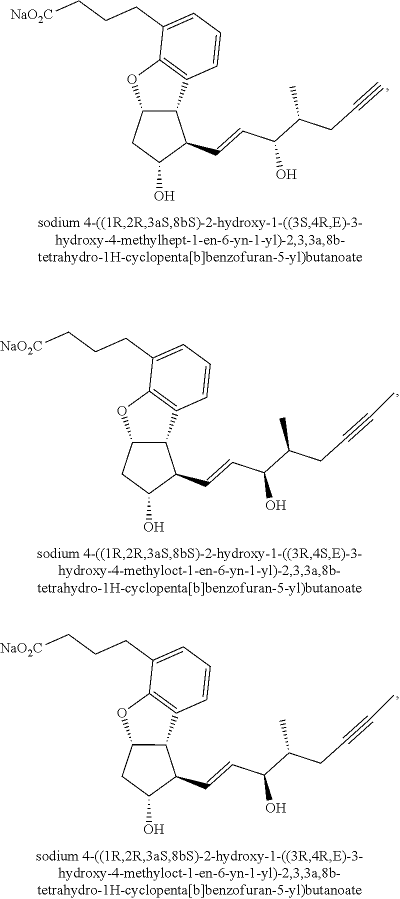

and a combination thereof.

The present disclosure also includes embodiments directed towards a composition comprising a compound of Formula I, or a pharmaceutically acceptable salt thereof, prepared by a process comprising the steps of any methods for preparing a compound of Formula I as disclosed herein.

The present disclosure also includes embodiments directed to a compound of the formula:

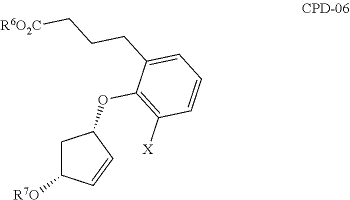

wherein
R⁶ is C₁₋₆ alkyl;
R⁷ is a hydroxy protecting group; and
X is halide.

In some embodiments, the compound of CPD-06 is:

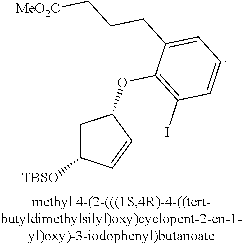 [SMILES: COC(=O)CCCc1cccc(I)c1O[C@@H]1C=C[C@H](O[Si](C)(C)C(C)(C)C)C1]

In some embodiments, Formula I is prepared without the use of chiral chromatography.

In some embodiments, sodium 4-((1R,2R,3aS,8bS)-2-hydroxy-1-((3S,4S,E)-3-hydroxy-4-methyloct-1-en-6-yn-1-yl)-2,3,3a,8b-tetrahydro-1H-cyclopenta[b]benzofuran-5-yl)butanoate is prepared without the use of chiral chromatography.

### Compositions

The present disclosure also includes embodiments directed to a composition comprising a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0%.

In some embodiments of the composition, the compound of Formula I is:

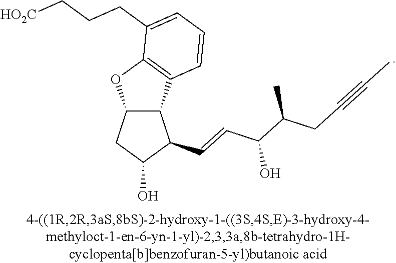 [SMILES: CC#CC[C@H](C)[C@H](O)/C=C/[C@@H]1[C@H]2c3cccc(CCCC(=O)O)c3O[C@H]2C[C@H]1O]

In some embodiments of the composition, the pharmaceutically acceptable salt of a compound of Formula I is:

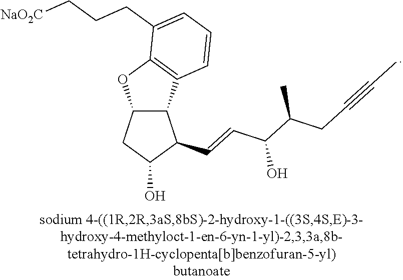

In some embodiments of the composition, the composition does not contain more than about 5.0% of an isomer other than the compound of Formula I, about 4.0% of an isomer other than the compound of Formula I, about 3.0% of an isomer other than the compound of Formula I, about 2.0% of an isomer other than the compound of Formula I, about 1.0% of an isomer other than the compound of Formula I, about 0.5% of an isomer other than the compound of Formula I, about 0.25% of an isomer other than the compound of Formula I, about 0.2% of an isomer other than the compound of Formula I, about 0.15% of an isomer other than the compound of Formula I, about 0.1% of an isomer other than the compound of Formula I, or about 0.05% of an isomer other than the compound of Formula I.

In some embodiments of the composition, the composition does not contain more than about 1.0% of an isomer other than the compound of Formula I.

In some embodiments of the composition, the composition does not contain more than about 0.15% of an isomer other than the compound of Formula I.

In some embodiments of the composition, the composition does not contain more than about 0.1% of an isomer other than the compound of Formula I.

In some embodiments of the composition, the composition does not contain more than about 0.05% of an isomer other than the compound of Formula I.

In some embodiments of the composition, the compound of Formula I is at least about 91% pure, at least about 92% pure, at least about 93% pure, at least about 94% pure, at least about 95% pure, at least about 96% pure, at least about 97% pure, at least about 98% pure, at least about 99% pure, at least about 99.1% pure, at least about 99.2% pure, at least about 99.3% pure, at least about 99.4% pure, at least about 99.5% pure, at least about 99.6% pure, at least about 99.6% pure, at least about 99.7% pure, at least about 99.8% pure, or at least about 99.9% pure.

In some embodiments of the composition, the compound of Formula I is at least about 95% pure.

In some embodiments of the composition, the compound of Formula I is at least about 99% pure.

In some embodiments of the composition, the compound of Formula I is at least about 99.8% pure.

In some embodiments of the composition, any individual impurity is present in an amount of less than about 0.95%, less than about 0.9%, less than about 0.85%, less than about 0.8%, less than about 0.75%, less than about 0.7%, less than about 0.65%, less than about 0.6%, less than about 0.55%, less than about 0.5%, less than about 0.45%, less than about 0.4%, less than about 0.35%, less than about 0.3%, less than about 0.25%, less than about 0.2%, less than about 0.15%, less than about 0.1%, less than about 0.05%, or less than about 0.01%.

In some embodiments of the composition, any individual impurity is present in an amount of less than about 0.15%.

In some embodiments of the composition, any individual impurity is present in an amount of less than about 0.1%.

In some embodiments of the composition, any individual impurity is present in an amount of less than about 0.05%.

In some embodiments of the composition, the total amount of the impurities is in an amount of less than about 10.0%, less than about 9.0%, less than about 8.0%, less than about 7.0%, less than about 6.0%, less than about 5.0%, less than about 4.0%, less than about 3.0%, less than about 2.0%, less than about 1.0%, less than about 0.9%, less than about 0.85%, less than about 0.8%, less than about 0.75%, less than about 0.7%, less than about 0.65%, less than about 0.6%, less than about 0.55%, less than about 0.5%, less than about 0.45%, less than about 0.4%, less than about 0.35%, less than about 0.3%, less than about 0.25%, less than about 0.2%, less than about 0.15%, less than about 0.1%, less than about 0.05%, or less than about 0.01%.

In some embodiments of the composition, the total amount of the impurities is in an amount of less than about 10.0%.

In some embodiments of the composition, the total amount of the impurities is in an amount of less than about 5.0%.

In some embodiments of the composition, the total amount of the impurities is in an amount of less than about 1.0%.

In some embodiments of the composition, the total amount of the impurities is in an amount of less than about 1.0%.

In some embodiments of the composition, the total amount of the impurities is in an amount of less than about 0.2%.

In some embodiments of the composition, the impurity is selected from:

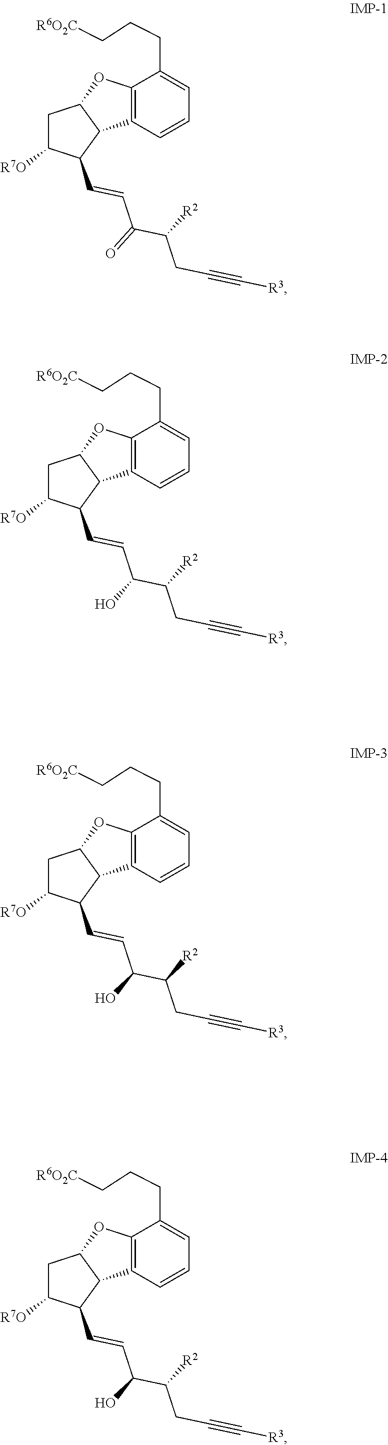

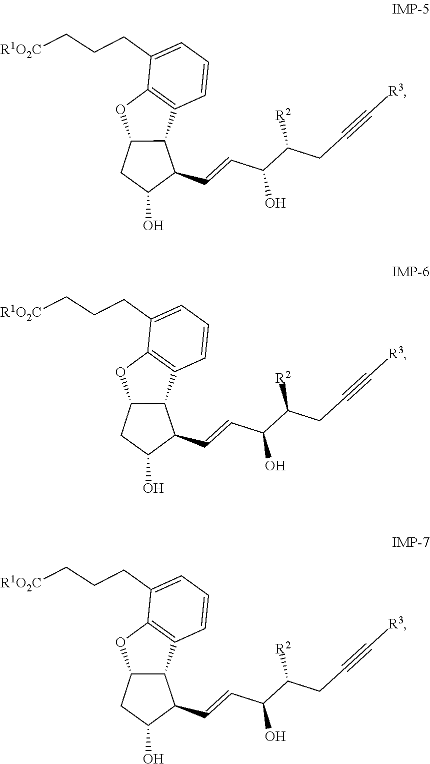

and a combination thereof.

In some embodiments of the composition, the impurity is selected from:

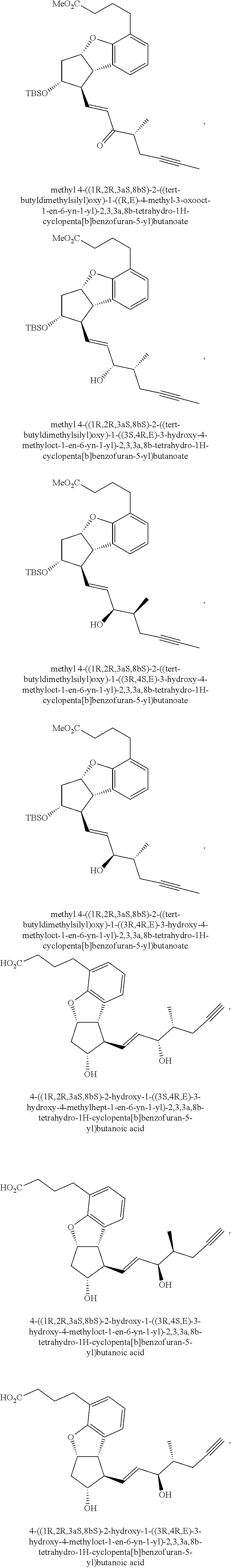

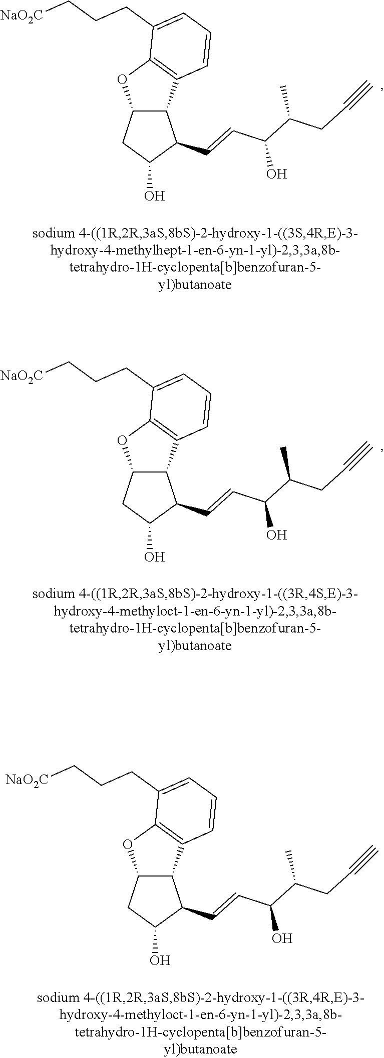

and a combination thereof.

In some embodiments of the composition, the composition contains not more than about 1.0 wt % degradation product, not more than about 0.9 wt % degradation product, not more than about 0.8 wt % degradation product, not more than about 0.7 wt % degradation product, not more than about 0.6 wt % degradation product, not more than about 0.5 wt % degradation product, not more than about 0.4 wt % degradation product, not more than about 0.3 wt % degradation product, not more than about 0.25 wt % degradation product, not more than about 0.2 wt % degradation product, not more than about 0.15 wt % degradation product, not more than about 0.1 wt % degradation product, not more than about 0.05 wt % degradation product, or not more than about 0.01 wt % degradation product.

In some embodiments of the composition, the composition contains not more than about 0.5 wt % degradation product.

In some embodiments of the composition, the composition contains not more than about 0.25 wt % degradation product.

In some embodiments of the composition, the composition contains not more than about 0.1 wt % degradation product.

A degradation product is an impurity resulting from a chemical change in the drug substance (i.e. a compound of Formula I) brought about during manufacture and/or storage of the new drug product by the effect of, for example, light, temperature, pH, water, or by reaction with an excipient and/or the immediate container closure system.

Guidelines for degradation products is disclosed in the Food and Drug Administration's Guidance for Industry Q3B® Impurities in New Drug Products (Revision 3) August 2006, which is hereby incorporated by reference in its entirety for all purposes.

The compositions can generally be in any physical form suitable for use in treating a subject. These forms can be referred to as a unit dosage form, such as an individual pill or tablet. In some embodiments, the compositions can be formulated as tablets, capsules, granules, powders, liquids, suspensions, gels, syrups, slurries, suppositories, patches, nasal sprays, aerosols, injectables, implantable sustained-release formulations, or mucoadherent films. In some embodiments, the composition may be formed as a tablet, a bi-layer tablet, a capsule, a multiparticulate, a drug coated sphere, a matrix tablet, or a multicore tablet. A physical form can be selected according to the desired method of treatment. In some embodiments, the physical form can be a liquid, for example for oral or IV, IP, IM, or IT administration.

Compositions can be manufactured by various conventional methods such as conventional mixing, dissolving, granulating, dragee-making, levigating, emulsifying, encapsulating, entrapping, or lyophilizing processes. Compositions can be formulated in a conventional manner using one or more physiologically acceptable carriers, diluents, excipients, or auxiliaries that facilitate processing of the active agent into preparations that can be used pharmaceutically. Proper formulation can be selected upon the route of administration chosen.

For oral administration, the compositions can combine the compound of Formula I, or a pharmaceutically acceptable salt thereof, with one or more pharmaceutically acceptable carriers well known in the art. Such carriers facilitate formulation as tablets, pills, dragees, capsules, liquids, gels, syrups, slurries, suspensions, and the like, for oral ingestion by a patient to be treated. For oral solid formulations such as, for example, powders, capsules and tablets, suitable excipients include fillers such as sugars, such as lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; and binding agents. If desired, disintegrating agents may be added, such as the cross-linked polyvinylpyrrolidone, agar, or alginic acid or a salt thereof such as sodium alginate. If desired, solid dosage forms may be sugar-coated or enteric-coated using standard techniques.

For oral liquid preparations such as, for example, suspensions, elixirs and solutions, suitable carriers, excipients, or diluents include water, glycols, oils, alcohols, etc. Additionally, flavoring agents, preservatives, coloring agents and the like can be added. For buccal administration, the compositions may take the form of tablets, lozenges, etc. formulated in conventional manner.

For administration by inhalation, the compositions can be delivered in the form of an aerosol spray from pressurized packs or a nebulizer, with the use of a suitable propellant, for example, dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, for example, gelatin for use in an inhaler or insufflator may be formulated containing a powder mix of the compound and a suitable powder base such as lactose or starch.

In some embodiments, the compositions are immediate release compositions, modified release compositions, or a combination thereof. In some embodiments, the immediate release composition releases the compound of Formula I, or a pharmaceutically acceptable salt thereof, within a short period of time after administration, typically less than about 4 hours, less than about 3.5 hours, less than about 3 hours, less than about 2.5 hours, less than about 2 hours, less than about 90 minutes, less than about 60 minutes, less than about 45 minutes, less than about 30 minutes, less than about 20 minutes, or less than about 10 minutes.

In some embodiments, the modified release composition may release the compound of Formula I, or a pharmaceutically acceptable salt thereof, at a sustained or controlled rate over an extended period of time or may release it after a lag time after administration. For example, it may be released from the composition 4 hours after administration, 8 hours after administration, 12 hours after administration, 16 hours after administration, or 24 hours after administration. Modified release compositions include extended release, sustained release, and delayed release compositions. In some embodiments, the modified release compositions may release about 10% in about 2 hours, about 20% in 2 hours, about 40% in about 2 hours, about 50% in about 2 hours, about 10% in about 3 hours, about 20% in 3 hours, about 40% in about 3 hours, about 50% in about 3 hours, about 10% in about 4 hours, about 20% in 4 hours, about 40% in about 4 hours, about 50% in about 4 hours, about 10% in about 6 hours, about 20% in 6 hours, about 40% in about 6 hours, or about 50% in about 6 hours.

In some embodiments, modified release compositions may comprise a matrix selected from microcrystalline cellulose, sodium carboxymethylcellulose, hydroxyalkylcelluloses such as hydroxy propyl methylcellulose and hydroxypropylcellulose, polyethylene oxide, alkylcelluloses such as methylcellulose and ethylcellulose, polyethylene glycol, polyvinylpyrrolidone, cellulose acetate, cellulose acetate butyrate, cellulose acetate phthalate, cellulose acetate trimellitate, polyvinyl acetate phthalate, polyalkylmethacrylates, polyvinyl acetate and mixtures thereof.

The modified release compositions can also be formulated as a depot preparation. Such long-acting formulations can be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

In some embodiments of the composition, the composition is formulated as a tablet, capsule, granule, powder, liquid, suspension, gel, syrup, slurry, suppository, patch, nasal spray, aerosol, injectable, implantable sustained-release formulation, or mucoadherent film.

In some embodiments of the composition, the composition is formulated as a tablet, a bi-layer tablet, a capsule, a multiparticulate, a drug coated sphere, a matrix tablet, or a multicore tablet.

In some embodiments of the composition, the composition is formulated as a tablet.

In some embodiments of the composition, the composition is formulated as a capsule.

In some embodiments of the composition, the composition comprises about 1 μg to about 500 μg of a compound of Formula I, about 1 μg to about 450 μg of a compound of Formula I, about 1 μg to about 400 μg of a compound of Formula I, about 1 μg to about 350 μg of a compound of Formula I, about 1 μg to about 300 μg of a compound of Formula I, about 1 μg to about 250 μg of a compound of Formula I, about 1 μg to about 200 μg of a compound of Formula I, about 1 μg to about 150 μg of a compound of Formula I, about 1 μg to about 100 μg of a compound of Formula I, about 1 μg to about 50 μg of a compound of Formula I, about 1 μg to about 25 μg of a compound of Formula I, about 1 μg to about 20 μg of a compound of Formula I, about 1 μg to about 15 μg of a compound of Formula I, about 1 μg to about 10 μg of a compound of Formula I, about 1 μg to about 5 μg of a compound of Formula I. In some embodiments of the composition, the composition comprises about 500 μg of a compound of Formula I, about 450 μg of a compound of Formula I, about 400 μg of a compound of Formula I, about 350 μg of a compound of Formula I, about 300 μg of a compound of Formula I, about 250 μg of a compound of Formula I, about 200 μg of a compound of Formula I, about 150 μg of a compound of Formula I, about 100 μg of a compound of Formula I, about 50 μg of a compound of Formula I, about 25 μg of a compound of Formula I, about 20 μg of a compound of Formula I, about 15 μg of a compound of Formula I, about 10 μg of a compound of Formula I, about 5 μg of a compound of Formula I, or a range between any two of these values.

In some embodiments of the composition, the composition comprises about 1 μg to about 500 μg of a compound of Formula I.

In some embodiments of the composition, the composition comprises about 20 μg of a compound of Formula I.

In some embodiments of the composition, the composition comprises about 10 μg of a compound of Formula I.

In some embodiments of the composition, the composition comprises about 5 μg of a compound of Formula I.

In some embodiments, the composition further comprises one or more pharmaceutically acceptable excipients. Examples of pharmaceutically acceptable excipients that may be present in the composition include but not limited to fillers/vehicles, solvents/co-solvents, preservatives, antioxidants, suspending agents, surfactants, antifoaming agents, buffering agents, chelating agents, sweeteners, flavoring agents, binders, extenders, disintegrants, diluents, lubricants, fillers, wetting agents, glidants, and combinations thereof.

In some embodiments, the composition further comprises lactose hydrate and corn starch. In some embodiments, hydroxypropyl cellulose, a water-soluble polymer, is used as a binder so that the main component is homogeneously combined with the additive during wet granulation. In some embodiments, the composition is a mixed solution comprising beraprost 314-d sodium salt with a water-soluble polymer in a solvent. In some embodiments, a mixed solution with only beraprost 314-d sodium dissolved is prepared by attaching or adsorbing the mixed solution to an additive. In some embodiments, the binder is a water-soluble polymer, such as hydroxypropylmethyl cellulose, polyoxyethylene polyoxypropylene glycol, polyethylene glycol, polyvinyl caprolactams, polyvinyl acetates, ethylcellulose, hydroxypropyl cellulose, polyethylene glycol graft polymer, aminoalkyl methacrylate copolymer, hydroxylpropyl cellulose, ethyl cellulose, polyvinylpyrrolidone, copovidone, polyethylene glycol hydrogenated castor oil, D-alpha-tocopheryl polyethylene glycol, dextrin, arabic gum, hydroxypropylmethylcellulose acetate succinate. In some embodiments, the composition is a coated tablet coated with water-soluble polymer such as a hydroxypropyl cellulose film, a polyvinyl alcohol, a hydroxypropyl methylcellulose, or combinations thereof.

In some embodiments, the composition can further comprise one or more exemplary fillers. Examples of exemplary fillers include cellulose and cellulose derivatives such as microcrystalline cellulose; starches such as dry starch, hydrolyzed starch, and starch derivatives such as corn starch; cyclodextrin; sugars such as powdered sugar and sugar alcohols such as lactose, mannitol, sucrose and sorbitol; inorganic fillers such as aluminum hydroxide gel, precipitated calcium carbonate, carbonate, magnesium aluminometasilicate, dibasic calcium phosphate; and sodium chloride, silicon dioxide, titanium dioxide, titanium oxide, dicalcium phosphate dihydrate, calcium sulfate, alumina, kaolin, talc, or combinations thereof. Fillers may be present in the composition from about 20 wt % to about 65 wt %, about 20 wt % to about 50 wt %, about 20 wt % to about 40 wt %, about 45 wt % to about 65 wt %, about 50 wt % to about 65 wt %, or about 55 wt % to about 65 wt % of the total weight of the composition, or any value between these ranges.

In some embodiments, the composition further comprises one or more disintegrants. Examples of disintegrants include starches, alginic acid, crosslinked polymers such as crosslinked polyvinylpyrrolidone, croscarmellose sodium, potassium starch glycolate, sodium starch glycolate, clays, celluloses, starches, gums, or combinations thereof. Disintegrants may be present in the composition from about 1 wt % to about 10 wt %, about 1 wt % to about 9 wt %, about 1 wt % to about 8 wt %, about 1 wt % to about 7 wt %, about 1 wt % to about 6 wt %, or about 1 wt % to about 5 wt % of the total weight of the composition, or any value between these ranges.

In some embodiments, the composition further comprises one or more binders, including but not limited to celluloses such as hydroxypropylcellulose, methyl cellulose, and hydroxypropylmethylcellulose; starches such as corn starch, pregelatinized starch, and hydroxpropyl starch; waxes and natural and synthetic gums such as acacia, tragacanth, sodium alginate; synthetic polymers such as polymethacrylates and polyvinylpyrrolidone; and povidone, dextrin, pullulane, agar, gelatin, tragacanth, macrogol, or combinations thereof. Binders may be present in the composition from about 0.5 wt % to about 5 wt %, about 0.5 wt % to about 4 wt %, about 0.5 wt % to about 3 wt %, about 0.5 wt % to about 2 wt %, or about 0.5 wt % to about 1 wt % of the total weight of the composition, or any value between these ranges.

In some embodiments, the composition further comprises one or more wetting agents, including but not limited to oleic acid, glyceryl monostearate, sorbitan mono-oleate, sorbitan monolaurate, triethanolamine oleate, polyoxyethylene sorbitan mono-oleate, polyoxyethylene sorbitan monolaurate, sodium oleate, sodium lauryl sulfate, poloxamers, poloxamer 188, polyoxyethylene ethers, polyoxyethylene sorbitan fatty acid esters, polyoxyethylene fatty acid esters, polyethylene glycol fatty acid esters, polyoxyethylene hardened castor oil, polyoxyethylene alkyl ethers, polysorbates, cetyl alcohol, glycerol fatty acid esters (for example, triacetin, glycerol monostearate, etc.), polyoxymethylene stearate, sodium lauryl sulfate, sorbitan fatty acid esters, sucrose fatty acid esters, benzalkonium chloride, polyethoxylated castor oil, and combinations thereof. Wetting agents may be present in the composition from about 0.1 wt % to about 1 wt %, about 0.1 wt % to about 2 wt %, about 0.1 wt % to about 3 wt %, about 0.1 wt % to about 4 wt %, or about 0.1 wt % to about 5 wt % of the total weight of the composition, or any value between these ranges.

In some embodiments, the composition further comprises one or more lubricants, including but not limited to stearic acid, magnesium stearate, calcium hydroxide, talc, corn starch, sodium stearyl fumarate, alkali-metal and alkaline earth metal salts, waxes, boric acid, sodium benzoate, sodium acetate, sodium chloride, leucine, polyethylene glycol (PEG), a methoxypolyethylene glycol, propylene glycol, sodium oleate, glyceryl behenate, glyceryl palmitostearate, glyceryl benzoate, magnesium lauryl sulfate, sodium lauryl sulfate, and combinations thereof. Lubricants may be present in the composition from about 0.1 wt % to about 5 wt %, about 0.1 wt % to about 4 wt %, about 0.1 wt % to about 3 wt %, about 0.1 wt % to about 2 wt %, or about 0.1 wt % to about 1 wt % of the total weight of the composition, or any value between these ranges.

In some embodiments, the composition further comprises one or more glidants, including but not limited to colloidal silicon dioxide, talc, sodium lauryl sulfate, native starch, and combinations thereof. Glidants may be present in the composition from about 0.05 wt % to about 1 wt %, about 0.05 wt % to about 0.9 wt %, about 0.05 wt % to about 0.8 wt %, about 0.05 wt % to about 0.5 wt %, or about 0.05 wt % to about 0.1 wt % of the total weight of the composition, or any value between these ranges.

In some embodiments, the composition is a tablet and further comprises a top coat, such as hydroxypropyl-methylcellulose coating or polyvinyl alcohol coating, and are available under the trade name Opadry, such as Opadry White, Opadry II (Opadry is a registered trademark of BPSI Holdings LLC, Wilmington, DE, USA). Top coats may be present in the composition from about 1 wt % to about 10 wt %, about 1 wt % to about 9 wt %, about 1 wt % to about 8 wt %, about 1 wt % to about 7 wt %, about 1 wt % to about 6 wt %, or about 1 wt % to about 5 wt % of the total weight of the composition, or any value between these ranges.

In some embodiments, the composition can further comprise one or more preservative agents. Examples of preservative agents include sodium benzoate, paraoxybenzoic acid esters, methyl, ethyl, butyl, and propyl parabens, chlorobutanol, benzyl alcohol, phenylethylalcohol, dehydroacetic acid, sorbic acid, benzalkonium chloride (BKC), benzethonium chloride, phenol, phenylmercuric nitrate, thimerosal, or combinations thereof. Preservative agents can be included in the liquid dosage form. The preservative agents can be in an amount sufficient to extend the shelf-life or storage stability, or both, of the liquid dosage form. Preservatives may be present in the composition from about 0.05 wt % to about 1 wt %, about 0.05 wt % to about 0.9 wt %, about 0.05 wt % to about 0.8 wt %, about 0.05 wt % to about 0.5 wt %, or about 0.05 wt % to about 0.1 wt % of the total weight of the composition, or any value between these ranges.

In some embodiments, the composition can further comprise one or more flavoring agents. Examples of flavoring agents include synthetic flavor oils and flavoring aromatics and/or natural oils, extracts from plants leaves, flowers, fruits, and so forth and the like or any combinations thereof. Additional examples include cinnamon oil, oil of wintergreen, peppermint oils, clove oil, bay oil, anise oil, eucalyptus, thyme oil, cedar leaf oil, oil of nutmeg, oil of sage, oil of bitter almonds, and cassia oil and the like or any combinations thereof. Also useful as flavors are vanilla, citrus oil, including lemon, orange, grape, lime and grapefruit, and fruit essences, including apple, banana, pear, peach, strawberry, raspberry, cherry, plum, pineapple, apricot, strawberry flavor, tutti-fruity flavor, mint flavor, or any combinations thereof. Flavoring agents may be present in the composition from about 0.1 wt % to about 5 wt %, about 0.1 wt % to about 4 wt %, about 0.1 wt % to about 3 wt %, about 0.1 wt % to about 2 wt %, or about 0.1 wt % to about 1 wt % of the total weight of the composition, or any value between these ranges.

In some embodiments, the composition can further comprise one or more antioxidants. Examples of antioxidants include flavonoids, anthocyanidins, anthocyanins, proanthocyanidins, or combinations thereof. Antioxidants may be present in the composition from about 0.05 wt % to about 1 wt %, about 0.05 wt % to about 0.9 wt %, about 0.05 wt % to about 0.8 wt %, about 0.05 wt % to about 0.5 wt %, or about 0.05 wt % to about 0.1 wt % of the total weight of the composition, or any value between these ranges.

### Methods of Use

The present disclosure also includes embodiments directed to a method of treating cancer in a subject, the method comprising administering at least one bispecific antibody and a first composition to the subject, wherein the first composition comprises at least an effective amount of a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0%.

In some embodiments of the method of treating cancer, the first composition does not contain more than about 5.0% of an isomer other than the compound of Formula I, about 4.0% of an isomer other than the compound of Formula I, about 3.0% of an isomer other than the compound of Formula I, about 2.0% of an isomer other than the compound of Formula I, about 1.0% of an isomer other than the compound of Formula I, about 0.5% of an isomer other than the compound of Formula I, about 0.25% of an isomer other than the compound of Formula I, about 0.2% of an isomer other than the compound of Formula I, about 0.15% of an isomer other than the compound of Formula I, about 0.1% of an isomer other than the compound of Formula I, or about 0.05% of an isomer other than the compound of Formula I.

In some embodiments of the method of treating cancer, the first composition does not contain more than about 1.0% of an isomer other than the compound of Formula I.

In some embodiments of the method of treating cancer, the first composition does not contain more than about 0.15% of an isomer other than the compound of Formula I.

In some embodiments of the method of treating cancer, the first composition does not contain more than about 0.1% of an isomer other than the compound of Formula I.

In some embodiments of the method of treating cancer, the first composition does not contain more than about 0.05% of an isomer other than the compound of Formula I.

In some embodiments of the method of treating cancer, the compound of Formula I is at least about 91% pure, at least about 92% pure, at least about 93% pure, at least about 94% pure, at least about 95% pure, at least about 96% pure, at least about 97% pure, at least about 98% pure, at least about 99% pure, at least about 99.1% pure, at least about 99.2% pure, at least about 99.3% pure, at least about 99.4% pure, at least about 99.5% pure, at least about 99.6% pure, at least about 99.6% pure, at least about 99.7% pure, at least about 99.8% pure, or at least about 99.9% pure.

In some embodiments of the method of treating cancer, the compound of Formula I is at least about 95% pure.

In some embodiments of the method of treating cancer, the compound of Formula I is at least about 99% pure.

In some embodiments of the method of treating cancer, the compound of Formula I is at least about 99.8% pure.

In some embodiments of the method of treating cancer, any individual impurity is present in an amount of less than about 0.95%, less than about 0.9%, less than about 0.85%, less than about 0.8%, less than about 0.75%, less than about 0.7%, less than about 0.65%, less than about 0.6%, less than about 0.55%, less than about 0.5%, less than about 0.45%, less than about 0.4%, less than about 0.35%, less than about 0.3%, less than about 0.25%, less than about 0.2%, less than about 0.15%, less than about 0.1%, less than about 0.05%, or less than about 0.01%.

In some embodiments of the method of treating cancer, any individual impurity is present in an amount of less than about 0.15%.

In some embodiments of the method of treating cancer, any individual impurity is present in an amount of less than about 0.1%.

In some embodiments of the method of treating cancer, any individual impurity is present in an amount of less than about 0.05%.

In some embodiments of the method of treating cancer, the first composition contains not more than about 1.0 wt % degradation product, not more than about 0.9 wt % degradation product, not more than about 0.8 wt % degradation product, not more than about 0.7 wt % degradation product, not more than about 0.6 wt % degradation product, not more than about 0.5 wt % degradation product, not more than about 0.4 wt % degradation product, not more than about 0.3 wt % degradation product, not more than about 0.25 wt % degradation product, not more than about 0.2 wt % degradation product, not more than about 0.15 wt % degradation product, not more than about 0.1 wt % degradation product, not more than about 0.05 wt % degradation product, or not more than about 0.01 wt % degradation product.

In some embodiments of the method of treating cancer, the first composition contains not more than about 0.5 wt % degradation product.

In some embodiments of the method of treating cancer, the first composition contains not more than about 0.25 wt % degradation product.

In some embodiments of the method of treating cancer, the first composition contains not more than about 0.1 wt % degradation product.

In some embodiments of the method of treating cancer, the compound of Formula I is:

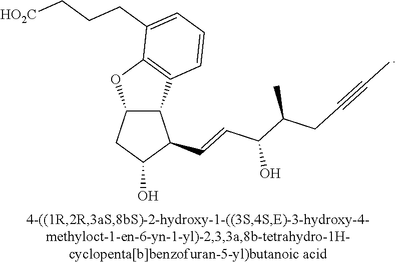 [SMILES: CC#CC[C@H](C)[C@H](O)/C=C/[C@@H]1[C@H]2c3cccc(CCCC(=O)O)c3O[C@H]2C[C@H]1O]

In some embodiments of the method of treating cancer, the pharmaceutically acceptable salt of a compound of Formula I is:

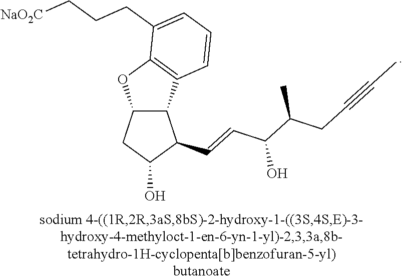

The methods can further comprise administering to the subject a second composition comprising at least one corticosteroid, tocilizumab, IL-6 receptor blocker, or combinations thereof. In some embodiments, the subject requires reduced treatment with the second composition relative to a subject who does not receive the first composition.

In some embodiments, the first composition reduces levels of one or more of cytokines of IL-1α, IL-1β, IL-2, IL-4, IL-6, IL-7, IL-8, IL-10, IL-12, IFN-γ, TNF-α, IP-10, MCP-1, MIP-1, RANTES, and GM-CSF in the subject.

In some embodiments, the first composition does not reduce a T cell-mediated killing of a cancer cell by more than about 5%.

The cancer can generally be any cancer suitable for treatment with bispecific antibody therapy. For example, the cancer can be B-cell lymphoma, aggressive, relapsed, or refractory diffuse large B cell lymphoma, primary mediastinal B-cell lymphoma, high grade B-cell lymphoma, transformed follicular lymphoma, relapsed or refractory mantle cell lymphoma, acute lymphoblastic leukemia, Hodgkin's lymphoma, non-Hodgkin's lymphoma, acute myeloid leukemia, or multiple myeloma. In some embodiments, the cancer is B-cell lymphoma. Other cancers suitable for treatment with bispecific antibody therapy include brain cancer, breast cancer, glioblastoma, lung cancer, non-small-cell lung cancer, multiple myeloma, ovarian cancer, neuroblastoma, colorectal, biliary, pancreatic, mesothelioma, hepatoblastoma, embryonal sarcoma, prostate, sarcoma, and liver metastases.

In some embodiments of the method of treating cancer, the subject is a mammal. In some embodiments of the method of treating cancer, the subject is a non-human primate, cat, dog, pig, cow, goat, horse, sheep, or rabbit. In some embodiments of the method of treating cancer, the subject is a human.

In some embodiments of the method of treating cancer, the at least one bispecific antibody and the first composition are administered to the subject concurrently.

In some embodiments of the method of treating cancer, the composition is administered to the subject after the at least one bispecific antibody is administered to the subject.

In some embodiments of the method of treating cancer, the first composition is administered to the subject starting about 1 day, about 2 days, about 3 days, about 4 days, about 5 days, about 6 days, or about 7 days after the at least one bispecific antibody is administered to the subject.

In some embodiments of the method of treating cancer, the first composition is administered to the subject starting about 3 days to about 7 days after the at least one bispecific antibody is administered to the subject.

In some embodiments of the method of treating cancer, the first composition is administered to the subject after the at least one bispecific antibody is administered to the subject; and once onset of CRS is detected.

In some embodiments of the method of treating cancer, the first is administered to the subject after the at least one bispecific antibody is administered to the subject; and once onset of CRS is detected by an increased level of one or more of cytokine IL-1α, IL-β, IL-2, IL-4, IL-6, IL-7, IL-8, IL-10, IL-12, IFN-γ, TNF-α, IP-10, MCP-1, MIP-1, RANTES, and GM-CSF.

In some embodiments of the method of treating cancer, the first composition is administered to the subject after the at least one bispecific antibody is administered to the subject; and once onset of CRS is detected by an increased level of one or more of cytokine IL-6, IL-10, IFN-γ, and TNF-α.

In some embodiments of the method of treating cancer, the first composition is administered for a period of about 1 day to about 30 days.

In some embodiments of the method of treating cancer, the first composition is administered for a period of about 7 days to about 14 days.

In some embodiments of the method of treating cancer, further comprises administering the first composition to the subject before administration of the at least one bispecific antibody.

In some embodiments of the method of treating cancer, the subject experiences reduced CRS relative to a similar subject receiving administered at least one bispecific antibody but not receiving the administered first composition.

In some embodiments of the method of treating cancer, the subject does not experience CRS.

In some embodiments of the method of treating cancer, the administering comprises oral delivery or intravenous injection (IV) delivery.

In some embodiments of the method of treating cancer, the at least one bispecific antibody has a mechanism of action of recruitment and activation of immune cells, blocking of immune checkpoints, blocking of inflammatory factors, blocking of dual signal pathways, or combinations thereof.

In some embodiments of the method of treating cancer, the at least one bispecific antibody is Blincyto (Blinatumomab), Hemlibra (emicizumab-kxwh), Rybrevant (amivantamab-vmjw), Kimmtrak (tebentafusp-tebn), or combinations thereof.

In some embodiments of the method of treating cancer, the at least one bispecific antibody is a CD19-CD3 bispecific antibody, a Factor IX-Factor X bispecific antibody, an EGFR-MET bispecific antibody, a gp100-CD3 bispecific antibody, a BCMA-CD3 bispecific antibody, a CD20-CD3 bispecific antibody, a GPRC5D-CD3 bispecific antibody, or combinations thereof.

The compounds and compositions described herein may be administered at therapeutically effective dosage levels to treat the recited conditions, disorders, and diseases.

The compounds and compositions described herein may be administered at prophylactically effective dosage levels to mitigate or prevent the recited conditions, disorders, and diseases.

An additional method of treating cytokine release syndrome, ICANS, or both, associated with bispecific antibody administration can include administering one or more bispecific antibodies and a first composition to the subject, wherein: the first composition comprises at least an effective amount of a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0%; and the subject experiences reduced ICANS effects, relative to a similar subject receiving bispecific antibody therapy but not receiving the first composition. ICANS can be assessed and graded using a cognitive assessment tool called the “Immune Effector Cell-associated Encephalopathy (ICE) Assessment” tool, level of consciousness, presence and severity of seizures, motor control impairment, and presence of cerebral edema. In one embodiment, the subject does not experience CRS such as, for example, when the first composition is administered concurrently with or prior to the at least one bispecific antibody.

A further method of treating cytokine release syndrome, ICANS, or both, associated with bispecific antibody administration in a subject can include administering one or more bispecific antibodies and a first composition to the subject, wherein: the first composition comprises at least an effective amount of a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0%; and the subject experiences reduced severity measurements relative to a similar subject receiving the bispecific antibodies but not receiving the first composition. Severity measurements can include event grades, event duration, event incidence, incidence of ICU or hospital stays, duration of ICU or hospital stays, onset timing, mortality, interference with antibiotics or other supportive medications, or combinations thereof. Additional severity measurements include use of supportive therapies, use of medical interventions, and use of intensive medical interventions such as intubation.

An additional method of treating cytokine release syndrome, ICANS, or both, associated with bispecific antibody administration in a subject can include administering one or more bispecific antibodies and a first composition to the subject, wherein: the first composition comprises at least an effective amount of a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0%; and the first composition is administered once onset of CRS is detected by an increased level of one or more of cytokine MIF, IL-5, IL-17A, IL-23, IFN-γ, CXCL9/MIG, GCSF, VEGF-A, and TGF-β. Alternatively or additionally, onset of CRS can be detected by an increased level of one or more of cytokines such as, CCL2, IL-2, IL-6, IL-8, IL-10, IFN-γ, TNF-α, CXCL9, CXCL-10, VEGF, CCL3, GCSF, and GMCSF The onset of CRS can be detected in multiple ways. For example, onset can be detected by measuring an increase of one or more cytokines or one or more inflammatory markers. Alternatively, onset of CRS can be measured by presentation of symptoms. Common symptoms include fever, hypotension, and hypoxia.

An additional method of treating cytokine release syndrome, ICANS, or both, associated with bispecific antibody administration in a subject can include administering one or more bispecific antibodies and a first composition to the subject, wherein: the first composition comprises at least an effective amount of a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0%; the subject experiences CRS, and/or ICANS; and the subject requires reduced treatment with at least one corticosteroid or a second composition relative to a similar subject receiving bispecific antibodies but not receiving the administered first composition. In some examples, the subject does not require treatment with at least one corticosteroid or a second composition.

Use of the described methods and compositions can result in a reduction or elimination of CRS. Reduction can be an improvement or resolution of undesirable physiological symptoms the patient subject is experiencing, a quantifiable reduction in one or more cytokine concentration, or both. The reduction can generally be reduced by any amount. For example, the reduction can be at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, and in an ideal situation, about 100% reduction (complete elimination of disease, symptom, or other undesired property). Reduction can be relative to the effect that would be observed with administration of the at least one bispecific antibody but without administration of the first composition.

Bispecific antibodies are typically delivered by infusion in one single administration, although multiple administrations are also possible. While CRS does not occur in all patients, about 45-95% of patients receiving bispecific antibody therapy do develop CRS. CRS typically has an onset within the first week and can typically occur with a median time of onset of about 24-48 hours (and a range of about 6 hours to about 9 days) post administration of bispecific antibodies. The first composition can be administered starting concurrently with the bispecific antibody (that is, no delay period), or starting after a delay period. In some examples, the first composition can additionally be administered one or more times prior to administration of the bispecific antibody. The delay period can be a predetermined period of time after administration of the bispecific antibody (such as about 1 day, about 2 days, about 3 days, about 4 days, about 5 days, about 6 days, about 7 days, about 8 days, about 9 days, about 10 days, about 11 days, about 12 days, about 13 days, about 14 days, or more, or ranges between any two of these values). Example ranges of the delay period include about 3 days to about 7 days, about 2 days to 5 days, about 3 days to 5 days, about 2 days to 5 days, about 4 days to 8 days, and so on. Alternatively, the delay period can last until onset of CRS is detected.

Various timings and sequences of administration of the first composition are possible. For example, the first composition can be administered prior to administration of the bispecific antibody therapy, on the same day as administration of the bispecific antibody therapy, after administration of the bispecific antibody therapy, and combinations thereof. For example, the first composition can be administered prior to administration of the bispecific antibody therapy, on the same day as administration of the bispecific antibody therapy, and after administration of the bispecific antibody therapy. In one specific example, the first composition can be administered one day prior to administration of the bispecific antibody therapy, and then continues for at least about 14 days.

Onset of CRS can be detected by generally any method, such as detecting fever, headache, anorexia, nausea, fatigue, myalgia, hypoxia, low blood pressure, hypotension, impaired coagulation, capillary leakage, tachycardia, organ system failure and so on. For example, a simple method to detect onset of CRS is detecting fever. Alternatively, onset of CRS can be detected by monitoring increased levels of one or more cytokines such as IL-lIa, IL-1, IL-2, IL-4, IL-6, IL-7, IL-8, IL-10, IL-12, IFN-γ, TNF-α, IP-10, MCP-1, MIP-1, RANTES, and GM-CSF. In some examples, onset of CRS can be detected by monitoring increased levels of one or more cytokines such as IL-6, IFN-γ, TNF-α, and IL-10. Alternatively or additionally, onset of CRS can be detected by an increased level of one or more of cytokine CCL2, IL-2, IL-6, IL-8, IL-10, IFN-γ, TNF-α, CXCL9, CXCL-10, VEGF, CCL3, GCSF, and GMCSF. In some examples, onset of CRS can be detected by monitoring increased levels of one or more cytokines such as IL-6, IFN-γ, TNF-α, and IL-10. Alternatively, or additionally, onset of CRS can be detected by monitoring increased levels of one or more cytokines such as MIF, IL-5, IL-17A, IL-23, IFN-γ, CXCL9/MIG, GCSF, VEGF-A, and TGF-β. The onset of CRS can be detected in multiple ways. For example, onset can be detected by measuring an increase of one or more cytokines or one or more inflammatory markers. Alternatively, onset of CRS can be measured by presentation of symptoms. Common symptoms include fever, hypotension, and hypoxia. In some embodiments, the first compositions can reduce the levels of one or more cytokines such as, but not limited to IL-lIa, IL-1, IL-2, IL-4, IL-6, IL-7, IL-8, IL-10, IL-12, IFN-γ, TNF-α, IP-10, MCP-1, MIP-1, RANTES, and GM-CSF.

For example, after the delay period or upon detecting onset of CRS, the first composition can be administered for about 1 day, about 2 days, about 3 days, about 4 days, about 5 days, about 6 days, about 7 days, about 8 days, about 9 days, about 10 days, about 11 days, about 12 days, about 13 days, about 14 days, or longer, or ranges between any two of these values. CRS typically is resolved in about one week but has been documented to persist for 30 days or beyond. In some examples, the first composition can be administered for more than about 14 days, such as about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or longer, or ranges between any two of these values. Example ranges include about 1 day to about 7 days, about 1 day to about 14 days, about 1 day to about 21 days, about 1 day to about 30 days, about 7 days to about 14 days, about 7 days to about 21 days, about 7 days to about 30 days, about 14 days to about 21 days, and about 21 days to about 30 days.

The treatments can generally be performed at any effective schedule. For example, the first compositions disclosed herein may be administered once, as needed, about once daily, about twice daily, about three times a day, about four times a day, about once a week, about twice a week, about three times a week, about four times a week, about five times a week, about six times a week, about seven times a week, about every other week, about every other day, or the like for one or more dosing cycles. It will be understood that the specific dose level and frequency of dosage for any particular subject can be varied and will depend upon a variety of factors including the species, age, body weight, general health, gender and diet of the subject, the mode and time of administration, rate of excretion, drug combination, and severity of the particular condition.

Administration may be performed by generally any method. Example delivery methods of administering include topical delivery, subcutaneous delivery, intravenous injection (IV) delivery, intramuscular injection (IM) delivery, intrathecal injection (IT) delivery, intraperitoneal injection (IP) delivery, transdermal delivery, subcutaneous delivery, oral delivery, transmucosal oral delivery, pulmonary delivery, inhalation delivery, intranasal delivery, buccal delivery, rectal delivery, vaginal delivery, and combinations thereof. In some examples, the administering comprises oral delivery, subcutaneous, inhalation, IV, or IM. In some examples, the at least one bispecific antibody and the first composition can be administered by the same method or by different methods.

### Bispecific Antibodies

One or more bispecific antibodies can be administered. For example, about 1, about 2, about 3, about 4, about 5, about 6, or more different bispecific antibodies can be used.

In some embodiments, the bispecific antibodies can be T cell-redirecting antibodies (TRABs) also herein referring to as T cell redirecting bispecific T cell Engager (BiTE). In some embodiments, TRABs can be a bispecific antibody consisting of CD3-binding portion that binds to T cells and cancer antigen, B cell or NK cell-binding arms. TRABs can exert a strong toxicity against cancer cells via the activation of T cells.

The at least one bispecific antibody can generally be any bispecific antibody. Specific examples of commercially available bispecific antibodies include Blincyto (Blinatumomab; Amgen; Thousand Oaks, CA, USA), Hemlibra (emicizumab-kxwh; Genentech; South San Francisco, CA, USA), and Rybrevant (amivantamab-vmjw; Janssen Pharmaceuticals; Beerse, Belgium).

In some embodiments, the bispecific antibody is a CD19-CD3 bispecific antibody, a Factor IX-Factor X bispecific antibody, an EGFR-MET bispecific antibody, a gp100-CD3 bispecific antibody, a BCMA-CD3 bispecific antibody, a CD20-CD3 bispecific antibody, a GPRC5D-CD3 bispecific antibody, or combinations thereof.

Bispecific antibodies can have one or more mechanisms of action. Example mechanisms of action include recruitment and activation of immune cells for example, T cells, blocking of immune checkpoints for example, PD1 and PDL1, blocking of inflammatory factors, blocking of dual signal pathways, and others.

In some embodiments, the bispecific antibodies can be immune checkpoints blocking antibodies, inflammatory factors blocking antibodies, dual signaling pathway blocking antibodies, and/or bispecific antibodies that block the recruitment and activation of T-cells.

Examples of clinical bispecific antibodies that recruit and activate immune cells include MGD011 (MacroGenics), AFM11 (Affimed), Blinatumomab (Amgen), AMG562 (Amgen), A-319 (Generon), RG7828 (Roche), REGN1979 (Regeneron), RG6026 (Roche), GEN3013 (Genmab), FBTA05 (Trion), Plamotamab (Xencor), AMG330 (Amgen), AMG673 (Amgen), AMV-564 (Amphivena Therapeutics), GEM333 (GEMoaB Monoclonals), GBR1342 (Glenmark Pharmaceuticals), AMG424 (Amgen), AMG420 (Amgen), AMG701 (Amgen), JNJ-64007957 (Janssen), EM801 (Celgene), PF-06863135 (Pfizer), REGN5458 (Regeneron), TNB-383B (AbbVie), APV0436 (Aptevo Therapeutics), MGD006 (MacroGenics), Xmab14045 (Xencor), JNJ-63709178 (Janssen), MCLA-117 (Merus), RG6160 (Genentech), AMG427 (Amgen), JNJ-64407564 (Janssen), AMG111 (Amgen), RG7802 (Roche), Solitomab (Amgen), Catumaxomab (Trion), Pasotuxizumab (Bayer), HPN-424 (Harpoon), AMG160 (Amgen), MOR209 (Aptevo Therapeutics), BAY2010112 (Bayer), Ertumaxomab (Trion), GBR1302 (Glenmark Pharmaceuticals), M802 (YZYBio), RG6194 (Genentech), PF06671008 (MacroGenics), MGD007 (MacroGenics), MGD009 (MacroGenics), AMG757 (Amgen), REGN4018 (Regeneron), AMG596 (Amgen), ERY974 (Chugai), Tidutamab (Xencor), huGD2-BsAb (Y-mAbs), MGD014 (Macrogenics), AFM13 (Affimed), GTB-3550 (GT Biopharma), Teclistamab-cqyv (Janssen), Mosunetuzumab-axgb (Genentech/Roche), Blinatumomab (Amgen), Elranatamab (Pfizer), Epcoritamab (GenMab/Abbvie), Glofitamab (Roche), Talquetamab (Janssen), Odronextamab (Regeneron), and TG-1801 (TG Therapeutics).

Examples of clinical bispecific antibodies that block immune checkpoints include XmAb23104 (Xencor), AK104 (Akesobio AU), MGD019 (Macrogenics), XmAb20717 (Xencor), MEDI5752 (AstraZeneca), MGD013 (Macrogenics), RG7769 (Roche), LY3434172 (Eli Lilly), FS118 (F-Star), KN046 (Alphamab), and LY3415244 (Eli Lilly).

Examples of clinical bispecific antibodies that block inflammatory factors include ATN103 (Ablynx), ALX0061 (Ablynx), ALX0761 (Ablynx), ALX0141 (Ablynx), SAR156597 (Sanofi), GSK2434735 (GlaxoSmithKline), RG7990 (Genentech), AMG570 (Amgen), LY3090106 (Eli), and MEDI7352 (Medimmune).

Examples of clinical bispecific antibodies that block dual signal pathways include ABT165 (AbbVie), ABL-001 (ABL Bio), Navicixizumab (Celgene/Oncomed), RG7221 (Roche), BI836880 (Ablynx), RO5520985 (Roche), JNJ-61186372 (Janssen R&D), EMB01 (Epimab Biotherapeutics), MCLA-158 (Merus), MCLA-128 (Merus), KN026 (Alphamab), MBS301 (Beijing Mabworks Biotech), ZW25 (Zymeworks), MP0274 (Molecular Partners AG), RG7386 (Roche), MGD010 (MacroGenics), RG7992 (Genentech), MEDI3902 (Medimmune), BI1034020 (Boehringer Ingelheim), and Emicizumab (Roche). While aspects of the technology are described in terms of bispecific antibodies, additional examples of the technology can include the use of multi-specific antibodies. Multi-specific antibodies can include bispecific, trispecific, tetraspecific, and so on, as opposed to traditional monospecific antibodies.

In some embodiments, administration of a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0% can reduce or eliminate hospitalization associated with the development of CRS, ICANS or a combination thereof. In some embodiments, methods of administering a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0% can transform bispecific antibody therapy from primarily an in-patient to outpatient treatment.

The present disclosure also includes embodiments directed to a method of treating cytokine release syndrome (CRS), immune effector cell-associated neurotoxicity syndrome (ICANS), or both associated with administration of at least one bispecific antibody in a subject, the method comprising administering at least one bispecific antibody to the subject, and administering a first composition to the subject; wherein the first composition comprises at least an effective amount of a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0%.

In some embodiments of the method of treating cytokine release syndrome (CRS), immune effector cell-associated neurotoxicity syndrome (ICANS), or both associated with administration of at least one bispecific antibody, the first composition does not contain more than about 1.0% of an isomer other than the compound of Formula I.

In some embodiments of the method of treating cytokine release syndrome (CRS), immune effector cell-associated neurotoxicity syndrome (ICANS), or both associated with administration of at least one bispecific antibody, the first composition does not contain more than about 0.15% of an isomer other than the compound of Formula I.

In some embodiments of the method of treating cytokine release syndrome (CRS), immune effector cell-associated neurotoxicity syndrome (ICANS), or both associated with administration of at least one bispecific antibody, the first composition does not contain more than about 0.1% of an isomer other than the compound of Formula I.

In some embodiments of the method of treating cytokine release syndrome (CRS), immune effector cell-associated neurotoxicity syndrome (ICANS), or both associated with administration of at least one bispecific antibody, the first composition does not contain more than about 0.05% of an isomer other than the compound of Formula I.

In some embodiments of the method of treating cytokine release syndrome (CRS), immune effector cell-associated neurotoxicity syndrome (ICANS), or both associated with administration of at least one bispecific antibody, the compound of Formula I is at least about 91% pure, at least about 92% pure, at least about 93% pure, at least about 94% pure, at least about 95% pure, at least about 96% pure, at least about 97% pure, at least about 98% pure, at least about 99% pure, at least about 99.1% pure, at least about 99.2% pure, at least about 99.3% pure, at least about 99.4% pure, at least about 99.5% pure, at least about 99.6% pure, at least about 99.6% pure, at least about 99.7% pure, at least about 99.8% pure, or at least about 99.9% pure.

In some embodiments of the method of treating cytokine release syndrome (CRS), immune effector cell-associated neurotoxicity syndrome (ICANS), or both associated with administration of at least one bispecific antibody, the compound of Formula I is at least about 95% pure.

In some embodiments of the method of treating cytokine release syndrome (CRS), immune effector cell-associated neurotoxicity syndrome (ICANS), or both associated with administration of at least one bispecific antibody, the compound of Formula I is at least about 99% pure.

In some embodiments of the method of treating cytokine release syndrome (CRS), immune effector cell-associated neurotoxicity syndrome (ICANS), or both associated with administration of at least one bispecific antibody, the compound of Formula I is at least about 99.8% pure.

In some embodiments of the method of treating cytokine release syndrome (CRS), immune effector cell-associated neurotoxicity syndrome (ICANS), or both associated with administration of at least one bispecific antibody, any individual impurity is present in an amount of less than about 0.95%, less than about 0.9%, less than about 0.85%, less than about 0.8%, less than about 0.75%, less than about 0.7%, less than about 0.65%, less than about 0.6%, less than about 0.55%, less than about 0.5%, less than about 0.45%, less than about 0.4%, less than about 0.35%, less than about 0.3%, less than about 0.25%, less than about 0.2%, less than about 0.15%, less than about 0.1%, less than about 0.05%, or less than about 0.01%.

In some embodiments of the method of treating cytokine release syndrome (CRS), immune effector cell-associated neurotoxicity syndrome (ICANS), or both associated with administration of at least one bispecific antibody, any individual impurity is present in an amount of less than about 0.15%.

In some embodiments of the method of treating cytokine release syndrome (CRS), immune effector cell-associated neurotoxicity syndrome (ICANS), or both associated with administration of at least one bispecific antibody, any individual impurity is present in an amount of less than about 0.1%.

In some embodiments of the method of treating cytokine release syndrome (CRS), immune effector cell-associated neurotoxicity syndrome (ICANS), or both associated with administration of at least one bispecific antibody, any individual impurity is present in an amount of less than about 0.05%.

In some embodiments of the method of treating cytokine release syndrome (CRS), immune effector cell-associated neurotoxicity syndrome (ICANS), or both associated with administration of at least one bispecific antibody, the first composition contains not more than about 1.0 wt % degradation product, not more than about 0.9 wt % degradation product, not more than about 0.8 wt % degradation product, not more than about 0.7 wt % degradation product, not more than about 0.6 wt % degradation product, not more than about 0.5 wt % degradation product, not more than about 0.4 wt % degradation product, not more than about 0.3 wt % degradation product, not more than about 0.25 wt % degradation product, not more than about 0.2 wt % degradation product, not more than about 0.15 wt % degradation product, not more than about 0.1 wt % degradation product, not more than about 0.05 wt % degradation product, or not more than about 0.01 wt % degradation product.

In some embodiments of the method of treating cytokine release syndrome (CRS), immune effector cell-associated neurotoxicity syndrome (ICANS), or both associated with administration of at least one bispecific antibody, the first composition contains not more than about 0.5 wt % degradation product.

In some embodiments of the method of treating cytokine release syndrome (CRS), immune effector cell-associated neurotoxicity syndrome (ICANS), or both associated with administration of at least one bispecific antibody, the first composition contains not more than about 0.25 wt % degradation product.

In some embodiments of the method of treating cytokine release syndrome (CRS), immune effector cell-associated neurotoxicity syndrome (ICANS), or both associated with administration of at least one bispecific antibody, the first composition contains not more than about 0.1 wt % degradation product.

In some embodiments of the method of treating cytokine release syndrome (CRS), immune effector cell-associated neurotoxicity syndrome (ICANS), or both associated with administration of at least one bispecific antibody, the compound of Formula I is:

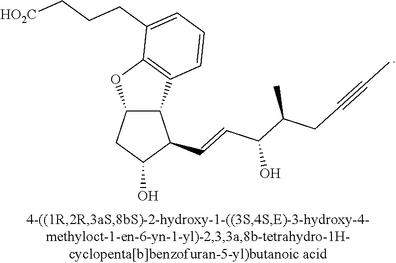 [SMILES: CC#CC[C@H](C)[C@H](O)/C=C/[C@@H]1[C@H]2c3cccc(CCCC(=O)O)c3O[C@H]2C[C@H]1O]

In some embodiments of the method of treating cytokine release syndrome (CRS), immune effector cell-associated neurotoxicity syndrome (ICANS), or both associated with administration of at least one bispecific antibody, the pharmaceutically acceptable salt of a compound of Formula I is:

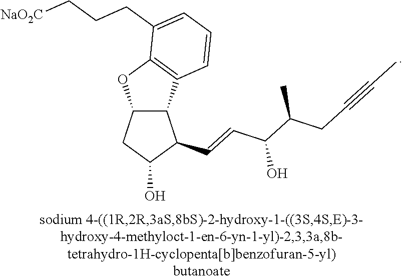

In some embodiments of the method of treating cytokine release syndrome (CRS), immune effector cell-associated neurotoxicity syndrome (ICANS), or both associated with administration of at least one bispecific antibody, further comprises administering to the subject a second composition comprising at least one corticosteroid, tocilizumab, IL-6 receptor blocker, or combinations thereof.

In some embodiments of the method of treating cytokine release syndrome (CRS), immune effector cell-associated neurotoxicity syndrome (ICANS), or both associated with administration of at least one bispecific antibody, further comprises administering to the subject a second composition comprising at least one corticosteroid, tocilizumab, IL-6 receptor blocker, or combinations thereof; wherein the subject requires reduced treatment with the second composition relative to a subject who does not receive the first composition.

In some embodiments of the method of treating cytokine release syndrome (CRS), immune effector cell-associated neurotoxicity syndrome (ICANS), or both, the first composition reduces levels of one or more of cytokines of IL-1α, IL-1β, IL-2, IL-4, IL-6, IL-7, IL-8, IL-10, IL-12, IFN-γ, TNF-α, IP-10, MCP-1, MIP-1, RANTES, and GM-CSF in the subject.

In some embodiments of the method of treating cytokine release syndrome (CRS), immune effector cell-associated neurotoxicity syndrome (ICANS), or both associated with administration of at least one bispecific antibody, the CRS is a grade 1 CRS, a grade 2 CRS, a grade 3 CRS or a grade 4 CRS.

In some embodiments of the method of treating cytokine release syndrome (CRS), immune effector cell-associated neurotoxicity syndrome (ICANS), or both associated with administration of at least one bispecific antibody, the ICANS is a grade 1 ICANS, a grade 2 ICANS, a grade 3 ICANS or a grade 4 ICANS.

In some embodiments of the method of treating cytokine release syndrome (CRS), immune effector cell-associated neurotoxicity syndrome (ICANS), or both associated with administration of at least one bispecific antibody, the subject experiences reduced severity measurements associated with CRS, ICANS or both upon receiving the first composition relative to subject who does not receive the first composition.

In some embodiments of the method of treating cytokine release syndrome (CRS), immune effector cell-associated neurotoxicity syndrome (ICANS), or both associated with administration of at least one bispecific antibody, the subject is a mammal. In some embodiments of the method of treating cytokine release syndrome (CRS), immune effector cell-associated neurotoxicity syndrome (ICANS), or both associated with administration of at least one bispecific antibody, the subject is a non-human primate, cat, dog, pig, cow, goat, horse, sheep, or rabbit. In some embodiments of the method of treating cytokine release syndrome (CRS), immune effector cell-associated neurotoxicity syndrome (ICANS), or both associated with administration of at least one bispecific antibody, the subject is a human.

In some embodiments of the method of treating cytokine release syndrome (CRS), immune effector cell-associated neurotoxicity syndrome (ICANS), or both associated with administration of at least one bispecific antibody, the at least one bispecific antibody and the first composition are administered to the subject concurrently.

In some embodiments of the method of treating cytokine release syndrome (CRS), immune effector cell-associated neurotoxicity syndrome (ICANS), or both associated with administration of at least one bispecific antibody, the first composition is administered to the subject after the at least one bispecific antibody is administered to the subject.

In some embodiments of the method of treating cytokine release syndrome (CRS), immune effector cell-associated neurotoxicity syndrome (ICANS), or both associated with administration of at least one bispecific antibody, the first composition is administered to the subject starting about 1 day, about 2 days, about 3 days, about 4 days, about 5 days, about 6 days, or about 7 days after the at least one bispecific antibody is administered to the subject.

In some embodiments of the method of treating cytokine release syndrome (CRS), immune effector cell-associated neurotoxicity syndrome (ICANS), or both associated with administration of at least one bispecific antibody, the first composition is administered to the subject starting about 3 days to about 7 days after the at least one bispecific antibody is administered to the subject.

In some embodiments of the method of treating cytokine release syndrome (CRS), immune effector cell-associated neurotoxicity syndrome (ICANS), or both associated with administration of at least one bispecific antibody, the first composition is administered to the subject after the at least one bispecific antibody is administered to the subject; and once onset of CRS is detected.

In some embodiments of the method of treating cytokine release syndrome (CRS), immune effector cell-associated neurotoxicity syndrome (ICANS), or both associated with administration of at least one bispecific antibody, the first composition is administered to the subject after the at least one bispecific antibody is administered to the subject; and once onset of CRS is detected by an increased level of one or more of cytokine IL-1α, IL-1β, IL-2, IL-4, IL-6, IL-7, IL-8, IL-10, IL-12, IFN-γ, TNF-α, IP-10, MCP-1, MIP-1, RANTES, and GM-CSF.

In some embodiments of the method of treating cytokine release syndrome (CRS), immune effector cell-associated neurotoxicity syndrome (ICANS), or both associated with administration of at least one bispecific antibody, the first composition is administered to the subject after the at least one bispecific antibody is administered to the subject; and once onset of CRS is detected by an increased level of one or more of cytokine IL-6, IL-10, IFN-γ, and TNF-α.

In some embodiments of the method of treating cytokine release syndrome (CRS), immune effector cell-associated neurotoxicity syndrome (ICANS), or both associated with administration of at least one bispecific antibody, once onset of CRS is detected by an increased level of one or more of cytokine IL-6, IL-10, IFN-γ, and TNF-α.

In some embodiments of the method of treating cytokine release syndrome (CRS), immune effector cell-associated neurotoxicity syndrome (ICANS), or both associated with administration of at least one bispecific antibody, the first composition is administered for a period of about 7 days to about 14 days.

In some embodiments of the method of treating cytokine release syndrome (CRS), immune effector cell-associated neurotoxicity syndrome (ICANS), or both associated with administration of at least one bispecific antibody, further comprises administering the first composition to the subject before administration of the at least one bispecific antibody.

In some embodiments of the method of treating cytokine release syndrome (CRS), immune effector cell-associated neurotoxicity syndrome (ICANS), or both associated with administration of at least one bispecific antibody, the subject experiences reduced CRS relative to a similar subject receiving administered at least one bispecific antibody but not receiving the administered first composition.

In some embodiments of the method of treating cytokine release syndrome (CRS), immune effector cell-associated neurotoxicity syndrome (ICANS), or both associated with administration of at least one bispecific antibody, the subject does not experience CRS.

In some embodiments of the method of treating cytokine release syndrome (CRS), immune effector cell-associated neurotoxicity syndrome (ICANS), or both associated with administration of at least one bispecific antibody, the administering comprises oral delivery or intravenous injection (IV) delivery.

In some embodiments of the method of treating cytokine release syndrome (CRS), immune effector cell-associated neurotoxicity syndrome (ICANS), or both associated with administration of at least one bispecific antibody, the at least one bispecific antibody has a mechanism of action of recruitment and activation of immune cells, blocking of immune checkpoints, blocking of inflammatory factors, blocking of dual signal pathways, or combinations thereof.

In some embodiments of the method of treating cytokine release syndrome (CRS), immune effector cell-associated neurotoxicity syndrome (ICANS), or both associated with administration of at least one bispecific antibody, the at least one bispecific antibody is Blincyto (Blinatumomab), Hemlibra (emicizumab-kxwh), Rybrevant (amivantamab-vmjw), Kimmtrak (tebentafusp-tebn), or combinations thereof.

In some embodiments of the method of treating cytokine release syndrome (CRS), immune effector cell-associated neurotoxicity syndrome (ICANS), or both associated with administration of at least one bispecific antibody, the at least one bispecific antibody is a CD19-CD3 bispecific antibody, a Factor IX-Factor X bispecific antibody, an EGFR-MET bispecific antibody, a gp100-CD3 bispecific antibody, a BCMA-CD3 bispecific antibody, a CD20-CD3 bispecific antibody, a GPRC5D-CD3 bispecific antibody or combinations thereof.

The present disclosure also includes embodiments directed to a method of treating cytokine release syndrome (CRS), immune effector cell-associated neurotoxicity syndrome (ICANS), or both associated with CAR T-cell administration in a subject, the method comprising administering to the subject a population of CAR T-cells, and a first composition; wherein the first composition comprises at least an effective amount of a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0%; and the first composition does not reduce a cell killing mediated by the population of CAR T-cells by more than about 5%.

In some embodiments of the method of treating cytokine release syndrome (CRS), immune effector cell-associated neurotoxicity syndrome (ICANS), or both associated with CAR T-cell administration, the first composition does not contain more than about 5.0% of an isomer other than the compound of Formula I, about 4.0% of an isomer other than the compound of Formula I, about 3.0% of an isomer other than the compound of Formula I, about 2.0% of an isomer other than the compound of Formula I, about 1.0% of an isomer other than the compound of Formula I, about 0.5% of an isomer other than the compound of Formula I, about 0.25% of an isomer other than the compound of Formula I, about 0.2% of an isomer other than the compound of Formula I, about 0.15% of an isomer other than the compound of Formula I, about 0.1% of an isomer other than the compound of Formula I, or about 0.05% of an isomer other than the compound of Formula I.

In some embodiments of the method of treating cytokine release syndrome (CRS), immune effector cell-associated neurotoxicity syndrome (ICANS), or both associated with CAR T-cell administration, the first composition does not contain more than about 1.0% of an isomer other than the compound of Formula I.

In some embodiments of the method of treating cytokine release syndrome (CRS), immune effector cell-associated neurotoxicity syndrome (ICANS), or both associated with CAR T-cell administration, the first composition does not contain more than about 0.15% of an isomer other than the compound of Formula I.

In some embodiments of the method of treating cytokine release syndrome (CRS), immune effector cell-associated neurotoxicity syndrome (ICANS), or both associated with CAR T-cell administration, the first composition does not contain more than about 0.1% of an isomer other than the compound of Formula I.

In some embodiments of the method of treating cytokine release syndrome (CRS), immune effector cell-associated neurotoxicity syndrome (ICANS), or both associated with CAR T-cell administration, the first composition does not contain more than about 0.05% of an isomer other than the compound of Formula I.

In some embodiments of the method of treating cytokine release syndrome (CRS), immune effector cell-associated neurotoxicity syndrome (ICANS), or both associated with CAR T-cell administration, the compound of Formula I is at least about 91% pure, at least about 92% pure, at least about 93% pure, at least about 94% pure, at least about 95% pure, at least about 96% pure, at least about 97% pure, at least about 98% pure, at least about 99% pure, at least about 99.1% pure, at least about 99.2% pure, at least about 99.3% pure, at least about 99.4% pure, at least about 99.5% pure, at least about 99.6% pure, at least about 99.6% pure, at least about 99.7% pure, at least about 99.8% pure, or at least about 99.9% pure.

In some embodiments of the method of treating cytokine release syndrome (CRS), immune effector cell-associated neurotoxicity syndrome (ICANS), or both associated with CAR T-cell administration, the compound of Formula I is at least about 95% pure.

In some embodiments of the method of treating cytokine release syndrome (CRS), immune effector cell-associated neurotoxicity syndrome (ICANS), or both associated with CAR T-cell administration, the compound of Formula I is at least about 99% pure.

In some embodiments of the method of treating cytokine release syndrome (CRS), immune effector cell-associated neurotoxicity syndrome (ICANS), or both associated with CAR T-cell administration, the compound of Formula I is at least about 99.8% pure.

In some embodiments of the method of treating cytokine release syndrome (CRS), immune effector cell-associated neurotoxicity syndrome (ICANS), or both associated with CAR T-cell administration, any individual impurity is present in an amount of less than about 0.95%, less than about 0.9%, less than about 0.85%, less than about 0.8%, less than about 0.75%, less than about 0.7%, less than about 0.65%, less than about 0.6%, less than about 0.55%, less than about 0.5%, less than about 0.45%, less than about 0.4%, less than about 0.35%, less than about 0.3%, less than about 0.25%, less than about 0.2%, less than about 0.15%, less than about 0.1%, less than about 0.05%, or less than about 0.01%.

In some embodiments of the method of treating cytokine release syndrome (CRS), immune effector cell-associated neurotoxicity syndrome (ICANS), or both associated with CAR T-cell administration, any individual impurity is present in an amount of less than about 0.15%.

In some embodiments of the method of treating cytokine release syndrome (CRS), immune effector cell-associated neurotoxicity syndrome (ICANS), or both associated with CAR T-cell administration, any individual impurity is present in an amount of less than about 0.1%.

In some embodiments of the method of treating cytokine release syndrome (CRS), immune effector cell-associated neurotoxicity syndrome (ICANS), or both associated with CAR T-cell administration, any individual impurity is present in an amount of less than about 0.05%.

In some embodiments of the method of treating cytokine release syndrome (CRS), immune effector cell-associated neurotoxicity syndrome (ICANS), or both associated with CAR T-cell administration, the first composition contains not more than about 1.0 wt % degradation product, not more than about 0.9 wt % degradation product, not more than about 0.8 wt % degradation product, not more than about 0.7 wt % degradation product, not more than about 0.6 wt % degradation product, not more than about 0.5 wt % degradation product, not more than about 0.4 wt % degradation product, not more than about 0.3 wt % degradation product, not more than about 0.25 wt % degradation product, not more than about 0.2 wt % degradation product, not more than about 0.15 wt % degradation product, not more than about 0.1 wt % degradation product, not more than about 0.05 wt % degradation product, or not more than about 0.01 wt % degradation product.

In some embodiments of the method of treating cytokine release syndrome (CRS), immune effector cell-associated neurotoxicity syndrome (ICANS), or both associated with CAR T-cell administration, the first composition contains not more than about 0.5 wt % degradation product.

In some embodiments of the method of treating cytokine release syndrome (CRS), immune effector cell-associated neurotoxicity syndrome (ICANS), or both associated with CAR T-cell administration, the first composition contains not more than about 0.25 wt % degradation product.

In some embodiments of the method of treating cytokine release syndrome (CRS), immune effector cell-associated neurotoxicity syndrome (ICANS), or both associated with CAR T-cell administration, the first composition contains not more than about 0.1 wt % degradation product.

In some embodiments of the method of treating cytokine release syndrome (CRS), immune effector cell-associated neurotoxicity syndrome (ICANS), or both associated with CAR T-cell administration, the compound of Formula I is:

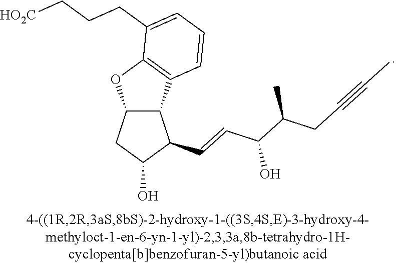 [SMILES: CC#CC[C@H](C)[C@H](O)/C=C/[C@@H]1[C@H]2c3cccc(CCCC(=O)O)c3O[C@H]2C[C@H]1O]

In some embodiments of the method of treating cytokine release syndrome (CRS), immune effector cell-associated neurotoxicity syndrome (ICANS), or both associated with CAR T-cell administration, the pharmaceutically acceptable salt of a compound of Formula I is:

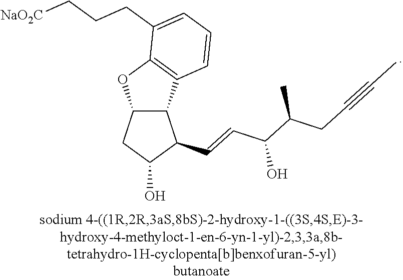

In some embodiments of the method of treating cytokine release syndrome (CRS), immune effector cell-associated neurotoxicity syndrome (ICANS), or both associated with CAR T-cell administration, the CRS is a grade 1 CRS, a grade 2 CRS, a grade 3 CRS or a grade 4 CRS.

In some embodiments of the method of treating cytokine release syndrome (CRS), immune effector cell-associated neurotoxicity syndrome (ICANS), or both associated with CAR T-cell administration, the ICANS is a grade 1 ICANS, a grade 2 ICANS, a grade 3 ICANS or a grade 4 ICANS.

In some embodiments of the method of treating cytokine release syndrome (CRS), immune effector cell-associated neurotoxicity syndrome (ICANS), or both associated with CAR T-cell administration, the first composition reduces levels of one or more of cytokines of IL-1α, IL-1β, IL-2, IL-4, IL-6, IL-7, IL-8, IL-10, IL-12, IFN-γ, TNF-α, IP-10, MCP-1, MIP-1, RANTES, and GM-CSF in the subject.

In some embodiments of the method of treating cytokine release syndrome (CRS), immune effector cell-associated neurotoxicity syndrome (ICANS), or both associated with CAR T-cell administration, further comprises administering a second composition comprising at least one corticosteroid, tocilizumab, IL-6 receptor blocker, or combinations thereof.

In some embodiments of the method of treating cytokine release syndrome (CRS), immune effector cell-associated neurotoxicity syndrome (ICANS), or both associated with CAR T-cell administration, the subject requires reduced treatment with the second composition relative to a subject who does not receive the first composition.

In some embodiments of the method of treating cytokine release syndrome (CRS), immune effector cell-associated neurotoxicity syndrome (ICANS), or both associated with CAR T-cell administration, the subject experiences reduced Parkinsonism effects upon receiving the first composition relative to a subject who does not receive the first composition.

In some embodiments of the method of treating cytokine release syndrome (CRS), immune effector cell-associated neurotoxicity syndrome (ICANS), or both associated with CAR T-cell administration, the subject experiences reduced severity measurements associated with CRS, ICANS or both upon receiving the first composition relative to subject who does not receive the first composition.

In some embodiments of the method of treating cytokine release syndrome (CRS), immune effector cell-associated neurotoxicity syndrome (ICANS), or both associated with CAR T-cell administration, the first composition is administered once onset of CRS is detected by an increased level of one or more cytokines of IL-6, IL-10, IFN-γ, TNF-α, MIF, IL-5, IL-17A, IL-23, CXCL9/MIG, GCSF, VEGF-A, and TGF-β, or one or more of inflammatory biomarker C-reactive protein (CRP) and ferritin.

In some embodiments of the method of treating cytokine release syndrome (CRS), immune effector cell-associated neurotoxicity syndrome (ICANS), or both associated with CAR T-cell administration, the CAR T-cell administration is performed to treat cancer; and the cancer is B-cell lymphoma, aggressive, relapsed or refractory diffuse large B cell lymphoma, primary mediastinal B-cell lymphoma, high grade B-cell lymphoma, transformed follicular lymphoma, relapsed or refractory mantle cell lymphoma, acute lymphoblastic leukemia, Hodgkin lymphoma, non-Hodgkin lymphoma, acute myeloid leukemia, or multiple myeloma. In some embodiments the cancer is B-cell lymphoma.

In some embodiments of the method of treating cytokine release syndrome (CRS), immune effector cell-associated neurotoxicity syndrome (ICANS), or both associated with CAR T-cell administration, the CAR T-cell administration is performed to treat cancer; and the cancer is brain cancer, breast cancer, glioblastoma, lung cancer, non-small-cell lung cancer, multiple myeloma, ovarian cancer, neuroblastoma, colorectal, biliary, pancreatic, mesothelioma, hepatoblastoma, embryonal sarcoma, prostate, sarcoma, or liver metastases.

In some embodiments of the method of treating cytokine release syndrome (CRS), immune effector cell-associated neurotoxicity syndrome (ICANS), or both associated with CAR T-cell administration, the subject is a mammal.

In some embodiments of the method of treating cytokine release syndrome (CRS), immune effector cell-associated neurotoxicity syndrome (ICANS), or both associated with CAR T-cell administration, the subject is a non-human primate, cat, dog, pig, cow, goat, horse, sheep, or rabbit.

In some embodiments of the method of treating cytokine release syndrome (CRS), immune effector cell-associated neurotoxicity syndrome (ICANS), or both associated with CAR T-cell administration, the subject is a human.

In some embodiments of the method of treating cytokine release syndrome (CRS), immune effector cell-associated neurotoxicity syndrome (ICANS), or both associated with CAR T-cell administration, the first composition is administered to the subject before the population of CAR T-cells are administered to the subject.

In some embodiments of the method of treating cytokine release syndrome (CRS), immune effector cell-associated neurotoxicity syndrome (ICANS), or both associated with CAR T-cell administration, the second composition is administered to the subject after the population of CAR T-cells are administered to the subject.

In some embodiments of the method of treating cytokine release syndrome (CRS), immune effector cell-associated neurotoxicity syndrome (ICANS), or both associated with CAR T-cell administration, the first composition and the second composition are administered to the subject after the population of CAR T-cells are administered to the subject.

In some embodiments of the method of treating cytokine release syndrome (CRS), immune effector cell-associated neurotoxicity syndrome (ICANS), or both associated with CAR T-cell administration, the first composition and the second composition are administered concurrently.

In some embodiments of the method of treating cytokine release syndrome (CRS), immune effector cell-associated neurotoxicity syndrome (ICANS), or both associated with CAR T-cell administration, the first composition and the second composition are administered to the subject before the population of CAR T-cells are administered to the subject.

In some embodiments of the method of treating cytokine release syndrome (CRS), immune effector cell-associated neurotoxicity syndrome (ICANS), or both associated with CAR T-cell administration, the first composition is administered to the subject starting about 1 day, about 2 days, about 3 days, about 4 days, about 5 days, about 6 days, or about 7 days after the CAR T-cells are administered to the subject.

In some embodiments of the method of treating cytokine release syndrome (CRS), immune effector cell-associated neurotoxicity syndrome (ICANS), or both associated with CAR T-cell administration, the first composition is administered to the subject starting about 3 days to about 7 days after the population of CAR T-cells are administered to the subject.

In some embodiments of the method of treating cytokine release syndrome (CRS), immune effector cell-associated neurotoxicity syndrome (ICANS), or both associated with CAR T-cell administration, the first composition is administered for a period of about 1 day to about 30 days.

In some embodiments of the method of treating cytokine release syndrome (CRS), immune effector cell-associated neurotoxicity syndrome (ICANS), or both associated with CAR T-cell administration, the first composition is administered for a period of about 7 days to about 14 days.

In some embodiments of the method of treating cytokine release syndrome (CRS), immune effector cell-associated neurotoxicity syndrome (ICANS), or both associated with CAR T-cell administration, the first composition is administered starting one day before administration of the CAR T-cells and continued for a period of at least about 14 days.

In some embodiments of the method of treating cytokine release syndrome (CRS), immune effector cell-associated neurotoxicity syndrome (ICANS), or both associated with CAR T-cell administration, the CAR T-cells are autologous CAR T-cells.

In some embodiments of the method of treating cytokine release syndrome (CRS), immune effector cell-associated neurotoxicity syndrome (ICANS), or both associated with CAR T-cell administration, the CAR T-cells are allogeneic CAR T-cells.

In some embodiments of the method of treating cytokine release syndrome (CRS), immune effector cell-associated neurotoxicity syndrome (ICANS), or both associated with CAR T-cell administration, the first composition further comprises at least one excipient, at least one filler, at least one disintegrant, at least one binder, at least one wetting agent, at least one lubricant, at least one glidant, at least one preservative agent, at least one flavoring agent, at least one antioxidant, or combinations thereof.

In some embodiments of the method of treating cytokine release syndrome (CRS), immune effector cell-associated neurotoxicity syndrome (ICANS), or both associated with CAR T-cell administration, the first composition is formulated as a tablet, capsule, granule, powder, liquid, suspension, gel, syrup, slurry, suppository, patch, nasal spray, aerosol, injectable, implantable sustained-release formulation, or mucoadherent film.

In some embodiments of the method of treating cytokine release syndrome (CRS), immune effector cell-associated neurotoxicity syndrome (ICANS), or both associated with CAR T-cell administration, the administering comprises topical delivery, subcutaneous delivery, intravenous injection (IV) delivery, intramuscular injection (IM) delivery, intrathecal injection (IT) delivery, intraperitoneal injection (IP) delivery, transdermal delivery, subcutaneous delivery, oral delivery, transmucosal oral delivery, pulmonary delivery, inhalation delivery, intranasal delivery, buccal delivery, rectal delivery, vaginal delivery, or combinations thereof.

In some embodiments of the method of treating cytokine release syndrome (CRS), immune effector cell-associated neurotoxicity syndrome (ICANS), or both associated with CAR T-cell administration, the administering comprises oral delivery or intravenous injection (IV) delivery.

In some embodiments of the method of treating cytokine release syndrome (CRS), immune effector cell-associated neurotoxicity syndrome (ICANS), or both associated with CAR T-cell administration, the compound of Formula I, or pharmaceutically acceptable salt thereof is present in a unit dose of the first composition in an amount of about 1 microgram to about 100 micrograms.

In some embodiments of the method of treating cytokine release syndrome (CRS), immune effector cell-associated neurotoxicity syndrome (ICANS), or both associated with CAR T-cell administration, the administering comprises delivering the first composition to the subject at an amount of a compound of Formula I, or a pharmaceutically acceptable salt thereof, at least about 0.1 microgram.

In some embodiments of the method of treating cytokine release syndrome (CRS), immune effector cell-associated neurotoxicity syndrome (ICANS), or both associated with CAR T-cell administration, the administering comprises delivering the first composition to the subject at an amount of a compound of Formula I, or a pharmaceutically acceptable salt thereof at about 0.1 microgram to about 5000 micrograms.

In some embodiments of the method of treating cytokine release syndrome (CRS), immune effector cell-associated neurotoxicity syndrome (ICANS), or both associated with CAR T-cell administration, the administering comprises delivering the first composition to the subject at an amount of a compound of Formula I, or a pharmaceutically acceptable salt thereof at about 15 micrograms to about 90 micrograms.

In some embodiments of the method of treating cytokine release syndrome (CRS), immune effector cell-associated neurotoxicity syndrome (ICANS), or both associated with CAR T-cell administration, the administering comprises delivering the first composition to the subject at an amount of a compound of Formula I, or a pharmaceutically acceptable salt thereof at about 60 micrograms to about 360 micrograms.

In some embodiments of the method of treating cytokine release syndrome (CRS), immune effector cell-associated neurotoxicity syndrome (ICANS), or both associated with CAR T-cell administration, the population of CAR T-cells comprises a population of BCMA CAR-T cells, a population of CD19 CAR-T cells, a population of CD19-CD3 bispecific CAR-T cells, or combinations thereof.

In some embodiments, a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0%, can be used to treat CRS, ICANS or both. In some embodiments, the CRS or ICANS can be associated with CAR T-cell administration.

In some embodiments, a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0% does not interfere with cell killing mediated by the CAR T-cells. The cell killing can occur when CAR T-cells engage or interact with their corresponding antigen on a cancer which can result in the death of the cancer cells. In some embodiments, a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0% does not reduce or inhibit CAR T-cell mediated cell killing. In some embodiments, a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0% does not reduce CAR T-cell mediated killing by more than about 1%, about 2%, about 3%, about 4% about 5%, about 6%, about 7%, about 8%, about 9%, about 10%, about 0.01% to about 0.1%, about 0.1% to about 1%, about 1% to about 10%, about 10% to about 20%, about 0.5% to about 5%, about 5% to about 15%, about 15% to about 25% or more. In some embodiments, a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0% thereof does not inhibit or reduce CAR T-cell activation or proliferation.

In some embodiments, the CAR-T cell therapy are administered to a patient with cancer. The cancer can generally be any cancer suitable for treatment with CAR T-cell therapy. In some embodiments, the cancer can be a hematological malignancy. For example, the cancer can be B-cell lymphoma, aggressive, relapsed or refractory diffuse large B cell lymphoma (DLBCL), primary mediastinal B-cell lymphoma, high grade B-cell lymphoma, transformed follicular lymphoma, relapsed or refractory mantle cell lymphoma, acute lymphoblastic leukemia, Hodgkin lymphoma, non-Hodgkin lymphoma, acute myeloid leukemia, or multiple myeloma. In some examples, the cancer is B-cell lymphoma. In some embodiments, the cancer can be a solid tumor. Cancers suitable for treatment with CAR T-cell therapy include brain cancer, breast cancer, glioblastoma, lung cancer, non-small-cell lung cancer, multiple myeloma, ovarian cancer, neuroblastoma, colorectal, biliary, pancreatic, mesothelioma, hepatoblastoma, embryonal sarcoma, prostate, sarcoma, and liver metastases.

In some embodiments, a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0% can be used to treat grade 1 CRS, a grade 2 CRS, a grade 3 CRS or a grade 4 CRS. Treatment with a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0% can reduce the severity of CRS such that treatment can result in a higher grade CRS, for example, grade 4 CRS becoming a lower grade CRS, for example, grade 1. In some embodiments, the treatment with a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0% thereof can eliminate or prevent CRS. In some embodiments, grade 1 CRS can include fever of about 38° C. or more. In some embodiments, grade 1 can include nausea, fatigue, headache and can require hospitalization. In some embodiments, grade 2 CRS can include fever of about 38° C. or more and hypotension not requiring vasopressors. Grade 2 CRS can further include hypoxia or decreased oxygen requiring low-flow nasal cannula or blow-by oxygen. In some embodiments, grade 2 CRS can include hypotension, and/or organ toxicity. In some embodiments, grade 3 can include fever of about 38° C. or more and hypotension requiring vasopressors with or without vasopressin treatment. Grade 3 can further include, hypoxia requiring high-flow nasal cannula, facemask, nonrebreather mask, or Venturi mask. In some embodiments, grade 4 can include fever of about 38° C. or more and hypotension requiring multiple vasopressors (excluding vasopressin) and/or organ toxicity. Grade 4 can further include, hypoxia requiring positive pressure (CPAP, BiPAP, intubation, mechanical ventilation) and/or organ toxicity.

In some embodiments, a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0% can be used to treat grade 1 ICANS, a grade 2 ICANS, a grade 3 ICANS or a grade 4 ICANS. Treatment with a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0% can reduce the severity of CRS such that treatment can result in a higher grade ICANS, for example, grade 4 ICANS becoming a lower grade ICANS, for example, grade 1 ICANS. In some embodiments, the treatment with a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0% can eliminate or prevent ICANS. In some embodiments, grade 1 ICANS can have an immune effector cell-associated encephalopathy (ICE) score: about 7-9. In some embodiments, grade 1 ICANS can include the following: consciousness: awakens spontaneously; seizure: none; motor findings: none; elevated ICP/cerebral edema: none. In some embodiments, grade 2 ICANS can include an ICE score of about 3-6. In some embodiments, grade 2 ICANS can include the following: consciousness: awakens to voice; seizure: none; motor findings: none; elevated ICP/cerebral edema: none. In some embodiments, grade 3 ICANS can include an ICE score of about 0-2. In some embodiments, grade 3 ICANS can include the following: consciousness: awakens only to tactile stimulus; seizure: any clinical seizure that resolves rapidly or nonconvulsive seizures on EEG that resolve with intervention; motor findings: none; elevated ICP/cerebral edema: focal/local edema on neuroimaging. In some embodiments, grade 4 ICANS can include an ICE score of about 0 (that is, patient or subject is unable to perform ICE). Grade 4 ICANS can include the following parameters: consciousness: subject is unarousable or requires vigorous or repetitive tactile stimuli to arouse, stupor or coma; seizure: life-threatening prolonged seizure (>5 min); or repetitive clinical or electrical seizures without return to baseline in between; motor findings: deep focal motor weakness such as hemiparesis or paraparesis; elevated ICP/cerebral edema: diffuse cerebral edema on neuroimaging; decerebrate or decorticate posturing; or cranial nerve VI palsy; or papilledema; or Cushing's triad.

In some embodiments, a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0%, can reduce the levels of one or more cytokines. Non-limiting examples of cytokines whose levels can be reduced, include, IL-1α, IL-1β, IL-2, IL-4, IL-6, IL-7, IL-8, IL-10, IL-12, IFN-γ, TNF-α, IP-10, MCP-1, MIP-1, RANTES, and/or GM-CSF. In some embodiments, the levels of one or more cytokines can be reduced by about 5%, about 10%, about 15%, about 20%, about 25%, about 30%, about 40%, about 45%, about 50%, about 55%, about 60%, about 65%, about 70%, about 75%, about 80%, about 85%, about 90%, about 95%, or more. In some embodiments, the levels of one or more cytokines can be reduced by about 1-10%, about 5-15%, about 10-20%, about 15-25%, about 20-30%, about 25-35%, about 30-40%, about 35-45%, about 40-50%, about 45-55%, about 50-60%, about 55-65%, about 60-70%, about 65-75%, about 70-80%, about 75-85%, about 80-90%, about 85-95%, or about 90-100%.

A compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0%, or a pharmaceutically acceptable salt thereof can be used to reduce the levels of one or more cytokines that are known to increase from about 0-36 hours after CAR T-cell therapy. For example, a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0% can be used to reduce the levels of IL-2, IL-15 and/or MCP-1. In some embodiments, a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0% can be used to reduce the levels of one or more cytokines that are known to increase from about 2 days to 5 days after CAR T-cell therapy. For example, the cytokines can be IL-6, IL-8, IL-10, IFN-γ, or TNF-α.

In some methods, a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0% can be used in a combination therapy approach with other active pharmaceutical ingredients. For example, a method of treating cytokine release syndrome, ICANS, or both, associated with CAR T-cell administration in a subject can include administering CAR T-cells, a first composition, and a second composition to the subject, wherein: the first composition comprises at least an effective amount of a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0%; and the second composition comprises at least one corticosteroid, tocilizumab, IL-6 receptor blocker, or combinations thereof.

An additional method of treating cytokine release syndrome, ICANS, or both, associated with CAR T-cell administration can include administering CAR T-cells and a first composition to the subject, wherein: the first composition comprises at least an effective amount of a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0%; and the subject experiences reduced ICANS effects, Parkinsonism effects, or both, relative to a similar subject receiving administered CAR T-cells but not receiving the administered first composition. ICANS can be assessed and graded using a cognitive assessment tool called the “Immune Effector Cell-associated Encephalopathy (ICE) Assessment” tool, level of consciousness, presence and severity of seizures, motor control impairment, and presence of cerebral edema. Parkinsonism effects can be measured by various metrics including tremors, muscle stiffness, neurologic issues, psychomotor retardation, handwriting changes, and gait changes.

A further method of treating cytokine release syndrome, ICANS, or both, associated with CAR T-cell administration in a subject can include administering CAR T-cells and a first composition to the subject, wherein: the first composition comprises at least an effective amount of a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0%; and the subject experiences reduced severity measurements relative to a similar subject receiving administered CAR T-cells but not receiving the administered first composition. Severity measurements can include event grades, event duration, event incidence, incidence of ICU or hospital stays, duration of ICU or hospital stays, onset timing, mortality, interference with antibiotics or other supportive medications, or combinations thereof. Additional severity measurements include use of supportive therapies, use of medical interventions, and use of intensive medical interventions such as intubation.

An additional method of treating cytokine release syndrome, ICANS, or both, associated with CAR T-cell administration in a subject can include administering CAR T-cells and a first composition to the subject, wherein: the first composition comprises at least an effective amount of a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0%; and the first composition is administered once onset of CRS is detected by an increased level of one or more of cytokine MIF, IL-5, IL-17A, IL-23, IFN-γ, CXCL9/MIG, GCSF, VEGF-A, and TGF-β. Alternatively or additionally, onset of CRS can be detected by an increased level of one or more of cytokines such as, CCL2, IL-2, IL-6, IL-8, IL-10, IFN-γ, TNF-α, CXCL9, CXCL-10, VEGF, CCL3, GCSF, and GMCSF The onset of CRS can be detected in multiple ways. For example, onset can be detected by measuring an increase of one or more cytokines or one or more inflammatory markers. Alternatively, onset of CRS can be measured by presentation of symptoms. Common symptoms include fever, hypotension, and hypoxia.

An additional method of treating cytokine release syndrome, ICANS, or both, associated with CAR T-cell administration in a subject can include administering CAR T-cells and a first composition to the subject, wherein: the first composition comprises at least an effective amount of a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0%; the subject experiences CRS, ICANS, Parkinsonism effects, or a combination thereof; and the subject requires reduced treatment with at least one corticosteroid relative to a similar subject receiving administered CAR T-cells but not receiving the administered first composition. In some examples, the subject does not require treatment with at least one corticosteroid.

Use of the described methods, kits, and compositions can result in a reduction or elimination of CRS, ICANS, Parkinsonism, or combinations thereof. Reduction can be an improvement or resolution of undesirable physiological symptoms the patient subject is experiencing, a quantifiable reduction in one or more cytokine concentration, or both. The reduction can generally be reduced by any amount. For example, the reduction can be at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, and in an ideal situation, about 100% reduction (complete elimination of disease, symptom, or other undesired property). Reduction can be relative to the effect observed with administration of the CAR T-cells but without administration of the first composition.

CAR T-cells are typically delivered by infusion in one single administration, although multiple administrations are also possible. While CRS does not occur in all patients, about 50-100% of patients receiving CAR T-cell therapy develop CRS. CRS typically has an onset within the first week and can typically occur over the first two weeks post administration of CAR T-cells. The first composition can be administered starting concurrently with the CAR T-cells (that is, no delay period), or starting after a delay period. In some examples, the first composition can additionally be administered one or more times prior to administration of the CAR T-cells. The delay period can be a predetermined period of time after administration of the CAR T-cells (such as about 1 day, about 2 days, about 3 days, about 4 days, about 5 days, about 6 days, about 7 days, about 8 days, about 9 days, about 10 days, about 11 days, about 12 days, about 13 days, about 14 days, or more, or ranges between any two of these values). Example ranges of the delay period include about 3 days to 7 days, about 2 days to 5 days, about 3 days to 5 days, about 2 days to 5 days, about 4 days to 8 days, and so on. Alternatively, the delay period can last until onset of CRS is detected.

Various timings and sequences of administration of the first composition are possible. For example, the first composition can be administered prior to administration of the CAR T-cells, on the same day as administration of the CAR T-cells, after administration of the CAR T-cells, and combinations thereof. For example, the first composition can be administered prior to administration of the CAR T-cells, on the same day as administration of the CAR T-cells, and after administration of the CAR T-cells. In one specific example, the first composition can be administered one day prior to administration of the CAR T-cells, and then continues for at least about 14 additional days.

Onset of CRS can be detected by generally any method, such as detecting fever, headache, anorexia, nausea, fatigue, myalgia, hypoxia, low blood pressure (hypotension), impaired coagulation, capillary leakage, tachycardia, organ system failure and so on. For example, a simple method to detect onset of CRS is detecting fever. Alternatively, onset of CRS can be detected by monitoring increased levels of one or more cytokines such as IL-1α, IL-1β, IL-2, IL-4, IL-6, IL-7, IL-8, IL-10, IL-12, IFN-γ, TNF-α, IP-10, MCP-1, MIP-1, RANTES, and GM-CSF. Alternatively or additionally, onset of CRS can be detected by an increased level of one or more of cytokine CCL2, IL-2, IL-6, IL-8, IL-10, IFN-γ, TNF-α, CXCL9, CXCL-10, VEGF, CCL3, GCSF, and GMCSF. In some examples, onset of CRS can be detected by monitoring increased levels of one or more cytokines such as IL-6, IFN-γ, TNF-α, and IL-10. Alternatively or additionally, onset of CRS can be detected by monitoring increased levels of one or more cytokines such as MIF, IL-5, IL-17A, IL-23, IFN-γ, CXCL9/MIG, GCSF, VEGF-A, and TGF-β. The onset of CRS can be detected in multiple ways. For example, onset can be detected by measuring an increase of one or more cytokines or one or more inflammatory markers. Alternatively, onset of CRS can be measured by presentation of symptoms. Common symptoms include fever, hypotension, and hypoxia. In some embodiments, the first composition can reduce the levels of one or more cytokines such as, but not limited to IL-1α, IL-1β, IL-2, IL-4, IL-6, IL-7, IL-8, IL-10, IL-12, IFN-γ, TNF-α, IP-10, MCP-1, MIP-1, RANTES, and GM-CSF.

For example, after the delay period or upon detecting onset of CRS, the first composition can be administered for about 1 day, about 2 days, about 3 days, about 4 days, about 5 days, about 6 days, about 7 days, about 8 days, about 9 days, about 10 days, about 11 days, about 12 days, about 13 days, about 14 days, or longer, or ranges between any two of these values. CRS typically is resolved in about one week but has been documented to persist for about 30 days or beyond. In some examples, the first composition can be administered for more than about 14 days, such as about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or longer, or ranges between any two of these values. Example ranges include about 1 day to 7 days, about 1 day to about 14 days, about 1 day to about 21 days, about 1 day to about 30 days, about 7 days to about 14 days, about 7 days to about 21 days, about 7 days to about 30 days, about 14 days to about 21 days, and about 21 days to about 30 days. In one specific example, the first composition can be administered starting one day before administration of the CAR T-cells and continued for at least about 14 days.

The treatments can generally be performed at any effective schedule. For example, the first compositions disclosed herein may be administered once, as needed, about once daily, about twice daily, about three times a day, about four times a day, about once a week, about twice a week, about three times a week, about four times a week, about five times a week, about six times a week, about seven times a week, about every other week, about every other day, or the like for one or more dosing cycles. It will be understood that the specific dose level and frequency of dosage for any particular subject can be varied and will depend upon a variety of factors including the species, age, body weight, general health, gender and diet of the subject, the mode and time of administration, rate of excretion, drug combination, and severity of the particular condition.

Administration may be performed by generally any method. Example delivery methods of administering include topical delivery, subcutaneous delivery, intravenous injection (IV) delivery, intramuscular injection (IM) delivery, intrathecal injection (IT) delivery, intraperitoneal injection (IP) delivery, transdermal delivery, subcutaneous delivery, oral delivery, transmucosal oral delivery, pulmonary delivery, inhalation delivery, intranasal delivery, buccal delivery, rectal delivery, vaginal delivery, and combinations thereof. In some examples, the administering comprises oral delivery, subcutaneous, inhalation, IV, or IM.

In some examples, administration of at least an effective amount a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0%, or a pharmaceutically acceptable salt thereof can reduce or eliminate the need for treatment with at least one corticosteroid, tocilizumab, IL-6 receptor blocker therapeutic, or combinations thereof, relative to a similar subject receiving administered CAR T-cells but not receiving the administered first composition. In an ideal example, the subject does not require treatment with at least one corticosteroid, tocilizumab, or IL-6 receptor blocker therapeutic. In some examples, the subject does experience CRS, ICANS, Parkinsonism effects, or a combination thereof, but requires a reduced or eliminated dosage or administration of corticosteroid, tocilizumab, IL-6 receptor blocker therapeutic, or combinations thereof to achieve a similar (or superior) clinical effect as compared to a similar subject receiving administered CAR T-cells but not receiving the administered first composition.

The CAR T-cells can generally be any CAR T-cells. Examples of current commercial CAR T-cell preparations include Abecma (idecabtagene vicleucel), Breyanzi (isocabtagene maraleucel), Kymriah (tisagenlecleucel), Tecartus (brexucabtagene autoleucel), Yescarta (axicabtagene ciloleucel), and Carvykti (ciltacabtagene autoleucel).

In some embodiments, a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0% can reduce or eliminate hospitalization associated with the development of CRS, ICANS or a combination thereof. In some embodiments, methods of the disclosure using a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0% can transform CAR-T therapy from primarily an in-patient to outpatient treatment.

### Kits

The present disclosure also includes embodiments directed to a kit for the treatment of cancer, the kit comprising a first container containing a first composition comprising a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0%; a second container containing at least one bispecific antibody; and instructions for the administration of the first composition to the subject. In some embodiments, the kit for the treatment of cancer further comprises a third container containing at least one corticosteroid, tocilizumab, IL-6 receptor blocker, or combinations thereof. In some embodiments, the kit for the treatment of cancer further comprises a fourth container containing at least one solvent.

The present disclosure also includes embodiments directed to a kit for the treatment of cytokine release syndrome (CRS), immune effector cell-associated neurotoxicity syndrome (ICANS), or both in a subject, the kit comprising a first container containing a first composition comprising a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0%; a second container containing at least one bispecific antibody; and instructions for the administration of the first composition to the subject. In some embodiments, the kit for the treatment of cytokine release syndrome (CRS), immune effector cell-associated neurotoxicity syndrome (ICANS), or both in a subject further comprises a third container containing at least one corticosteroid, tocilizumab, IL-6 receptor blocker, or combinations thereof. In some embodiments, the kit for the treatment of cytokine release syndrome (CRS), immune effector cell-associated neurotoxicity syndrome (ICANS), or both in a subject further comprises a fourth container containing at least one solvent.

The present disclosure also includes embodiments directed to a kit for treating a subject with CAR T-cells, the kit comprising a first container containing a first composition comprising a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0%; a second container containing a second composition comprising at least one corticosteroid, tocilizumab, IL-6 receptor blocker, or combinations thereof; and instructions for the administration of the first composition, the second composition, and CAR T-cells to a subject. In some embodiments, the kit for treating a subject with CAR T-cells further comprises a third container containing CAR T-cells. In some embodiments, the kit for treating a subject with CAR T-cells further comprises a fourth container containing at least one solvent.

In some embodiments of any of the kits disclosed herein, the first composition does not contain more than about 5.0% of an isomer other than the compound of Formula I, about 4.0% of an isomer other than the compound of Formula I, about 3.0% of an isomer other than the compound of Formula I, about 2.0% of an isomer other than the compound of Formula I, about 1.0% of an isomer other than the compound of Formula I, about 0.5% of an isomer other than the compound of Formula I, about 0.25% of an isomer other than the compound of Formula I, about 0.2% of an isomer other than the compound of Formula I, about 0.15% of an isomer other than the compound of Formula I, about 0.1% of an isomer other than the compound of Formula I, or about 0.05% of an isomer other than the compound of Formula I.

In some embodiments of any of the kits disclosed herein, the first composition does not contain more than about 1.0% of an isomer other than the compound of Formula I.

In some embodiments of any of the kits disclosed herein, the first composition does not contain more than about 0.15% of an isomer other than the compound of Formula I.

In some embodiments of any of the kits disclosed herein, the first composition does not contain more than about 0.1% of an isomer other than the compound of Formula I.

In some embodiments of any of the kits disclosed herein, the first composition does not contain more than about 0.05% of an isomer other than the compound of Formula I.

In some embodiments of any of the kits disclosed herein, the compound of Formula I is at least about 91% pure, at least about 92% pure, at least about 93% pure, at least about 94% pure, at least about 95% pure, at least about 96% pure, at least about 97% pure, at least about 98% pure, at least about 99% pure, at least about 99.1% pure, at least about 99.2% pure, at least about 99.3% pure, at least about 99.4% pure, at least about 99.5% pure, at least about 99.6% pure, at least about 99.6% pure, at least about 99.7% pure, at least about 99.8% pure, or at least about 99.9% pure.

In some embodiments of any of the kits disclosed herein, the compound of Formula I is at least about 95% pure.

In some embodiments of any of the kits disclosed herein, the compound of Formula I is at least about 99% pure.

In some embodiments of any of the kits disclosed herein, the compound of Formula I is at least about 99.8% pure.

In some embodiments of any of the kits disclosed herein, any individual impurity is present in an amount of less than about 0.95%, less than about 0.9%, less than about 0.85%, less than about 0.8%, less than about 0.75%, less than about 0.7%, less than about 0.65%, less than about 0.6%, less than about 0.55%, less than about 0.5%, less than about 0.45%, less than about 0.4%, less than about 0.35%, less than about 0.3%, less than about 0.25%, less than about 0.2%, less than about 0.15%, less than about 0.1%, less than about 0.05%, or less than about 0.01%.

In some embodiments of any of the kits disclosed herein, any individual impurity is present in an amount of less than about 0.15%.

In some embodiments of any of the kits disclosed herein, any individual impurity is present in an amount of less than about 0.1%.

In some embodiments of any of the kits disclosed herein, any individual impurity is present in an amount of less than about 0.05%.

In some embodiments of any of the kits disclosed herein, the first composition contains not more than about 1.0 wt % degradation product, not more than about 0.9 wt % degradation product, not more than about 0.8 wt % degradation product, not more than about 0.7 wt % degradation product, not more than about 0.6 wt % degradation product, not more than about 0.5 wt % degradation product, not more than about 0.4 wt % degradation product, not more than about 0.3 wt % degradation product, not more than about 0.25 wt % degradation product, not more than about 0.2 wt % degradation product, not more than about 0.15 wt % degradation product, not more than about 0.1 wt % degradation product, not more than about 0.05 wt % degradation product, or not more than about 0.01 wt % degradation product.

In some embodiments of any of the kits disclosed herein, the first composition contains not more than about 0.5 wt % degradation product.

In some embodiments of any of the kits disclosed herein, the first composition contains not more than about 0.25 wt % degradation product.

In some embodiments of any of the kits disclosed herein, the first composition contains not more than about 0.1 wt % degradation product.

In some embodiments of any of the kits disclosed herein, the compound of Formula I is:

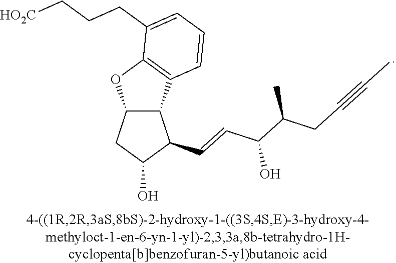 [SMILES: CC#CC[C@H](C)[C@H](O)/C=C/[C@@H]1[C@H]2c3cccc(CCCC(=O)O)c3O[C@H]2C[C@H]1O]

In some embodiments of any of the kits disclosed herein, the pharmaceutically acceptable salt of a compound of Formula I is:

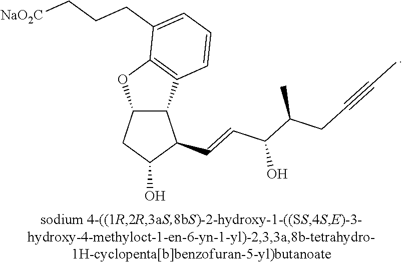

### Further Embodiments

Embodiment 1. A method of preparing a product comprising a compound of Formula I having the structure:

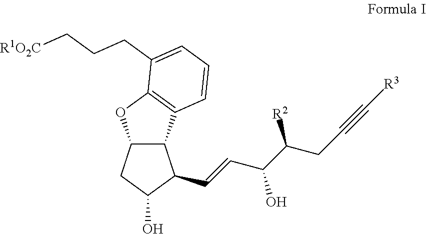

wherein
R¹ is selected from H and a cation;
R² and R³ are independently C₁₋₆ alkyl;
the method comprising the steps of:
(a) performing a condensation reaction on the compound

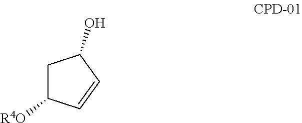

to form the compound

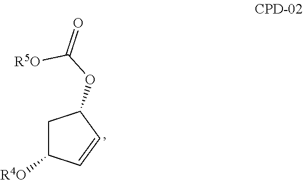

wherein R⁴ is C(O)C₁₋₆ alkyl and R⁵ is C₁₋₆ alkyl;
(b) coupling the carbonate of CPD-02 with

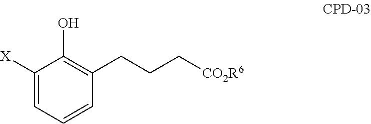

to form the compound

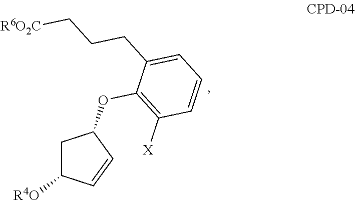

wherein is R⁶ is C₁₋₆ alkyl and X is halide;
(c) hydrolyzing CPD-04 to form the compound

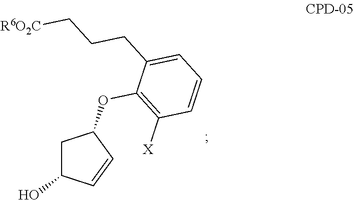

(d) protecting the alcohol of CPD-05 to form the compound

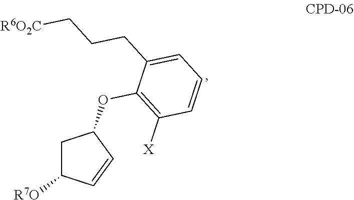

wherein R⁷ is a hydroxy protecting group;
(e) performing a radical cyclization and trapping reaction on CPD-06 with

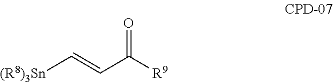

to form the compound

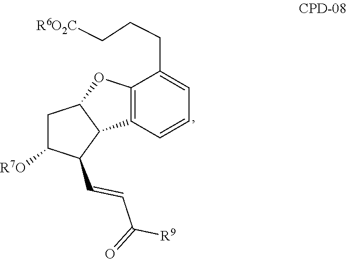

wherein R⁸ and R⁹ are independently C₁₋₆ alkyl; and
(f) converting CPD-08 to a compound of Formula I;
wherein the compound of Formula I is at least about 90% pure;
wherein the total amount of the impurities is in an amount of less than about 10.0%; and
wherein any individual impurity is present in an amount of less than about 1.0%.

Embodiment 2. The method of embodiment 1, further comprising cleaving the double bond of CPD-08 to from the compound

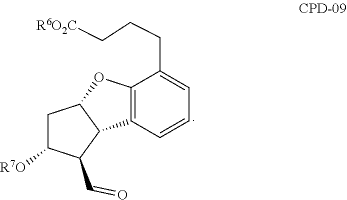

Embodiment 3. The method of embodiment 2, further comprising coupling CPD-09 with

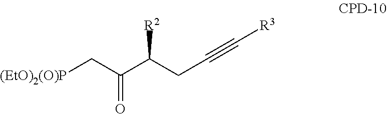

to form the compound

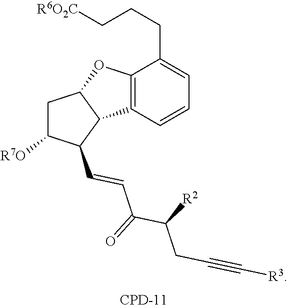

Embodiment 4. The method of embodiment 3, further comprising reduction of the ketone of CPD-11 to form the compound

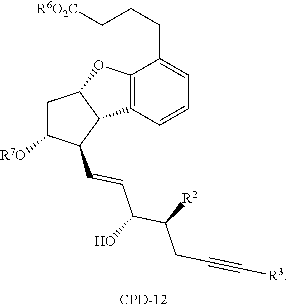

Embodiment 5. The method of embodiment 4, further comprising the deprotection of CPD-12 to form the compound

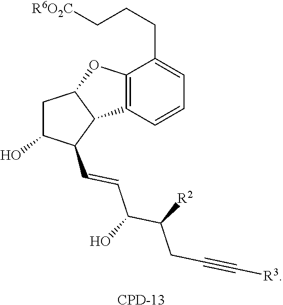

Embodiment 6. The method of embodiment 5, further comprising hydrolyzing CPD-13 to form the compound Formula I.

Embodiment 7. The method of embodiment 1, wherein R¹ is Na⁺.

Embodiment 8. The method of embodiment 1, wherein R², R³, and R⁶ are methyl.

Embodiment 9. The method of embodiment 1, wherein R⁴ is C(O)Me.

Embodiment 10. The method of embodiment 1, wherein R⁵ is ethyl.

Embodiment 11. The method of embodiment 1, wherein R⁷ is tert-butyldimethylsilyl.

Embodiment 12. The method of embodiment 1, wherein R⁸ is butyl.

Embodiment 13. The method of embodiment 1, wherein R⁹ is pentyl.

Embodiment 14. The method of embodiment 1, wherein the compound of Formula I is:

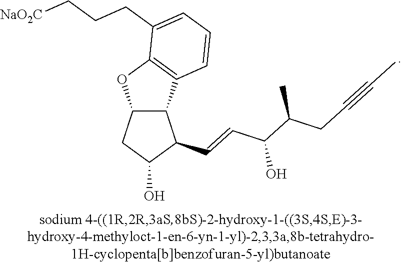

Embodiment 15. The method of embodiment 1, wherein the method does not produce more than about 1.0% of an isomer other than the compound of Formula I.

Embodiment 16. The method of embodiment 1, wherein the method does not produce more than about 0.15% of an isomer other than the compound of Formula I.

Embodiment 17. The method of embodiment 1, wherein the method does not produce more than about 0.1% of an isomer other than the compound of Formula I.

Embodiment 18. The method of embodiment 1, wherein the method does not produce more than about 0.05% of an isomer other than the compound of Formula I.

Embodiment 19. The method of embodiment 1, wherein the compound of Formula I is at least about 95% pure.

Embodiment 20. The method of embodiment 1, wherein the compound of Formula I is at least about 99% pure.

Embodiment 21. The method of embodiment 1, wherein the compound of Formula I is at least about 99.8% pure.

Embodiment 22. The method of embodiment 1, wherein any individual impurity is present in an amount of less than about 0.15%.

Embodiment 23. The method of embodiment 1, wherein any individual impurity is present in an amount of less than about 0.1%.

Embodiment 24. The method of embodiment 1, wherein any individual impurity is present in an amount of less than about 0.05%.

Embodiment 25. The method of embodiment 1, wherein the compound of Formula I is prepared in an overall yield of at least about 5%.

Embodiment 26. The method of embodiment 1, wherein the compound of Formula I is prepared in an overall yield of at least about 10%.

Embodiment 27. The method of embodiment 1, wherein the total amount of the impurities is in an amount of less than about 5.0%.

Embodiment 28. The method of embodiment 1, wherein the total amount of the impurities is in an amount of less than about 1.0%.

Embodiment 29. The method of embodiment 1, wherein the total amount of the impurities is in an amount of less than about 0.2%.

Embodiment 30. The method of embodiment 1, wherein the impurity is selected from:

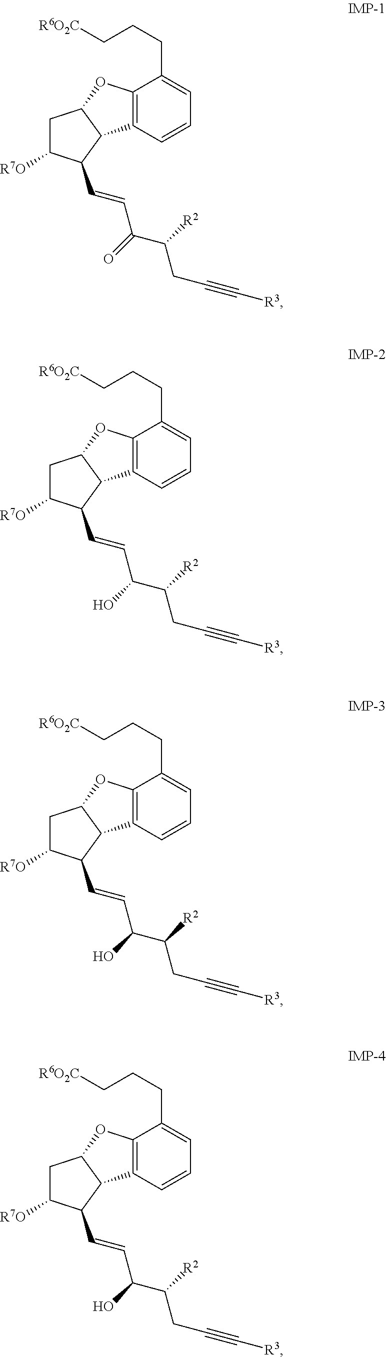

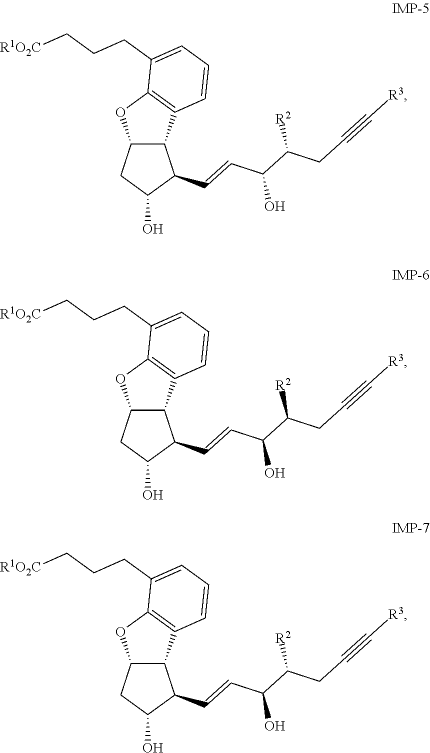

and a combination thereof.

Embodiment 31. The method of embodiment 1, wherein the impurity is selected from:

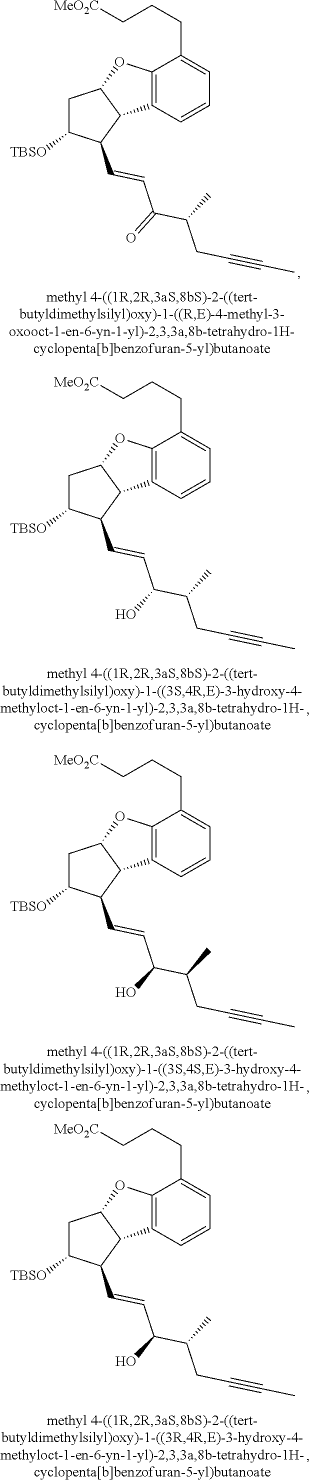 [SMILES: CC#CC[C@@H](C)C(=O)/C=C/[C@@H]1[C@H]2c3cccc(CCCC(=O)OC)c3O[C@H]2C[C@H]1O[Si](C)(C)C(C)(C)C ; CC#CC[C@@H](C)[C@H](O)/C=C/[C@@H]1[C@H]2c3cccc(CCCC(=O)OC)c3O[C@H]2C[C@H]1O[Si](C)(C)C(C)(C)C ; CC#CC[C@H](C)[C@@H](O)/C=C/[C@@H]1[C@H]2c3cccc(CCCC(=O)OC)c3O[C@H]2C[C@H]1O[Si](C)(C)C(C)(C)C ; CC#CC[C@@H](C)[C@@H](O)/C=C/[C@@H]1[C@H]2c3cccc(CCCC(=O)OC)c3O[C@H]2C[C@H]1O[Si](C)(C)C(C)(C)C]

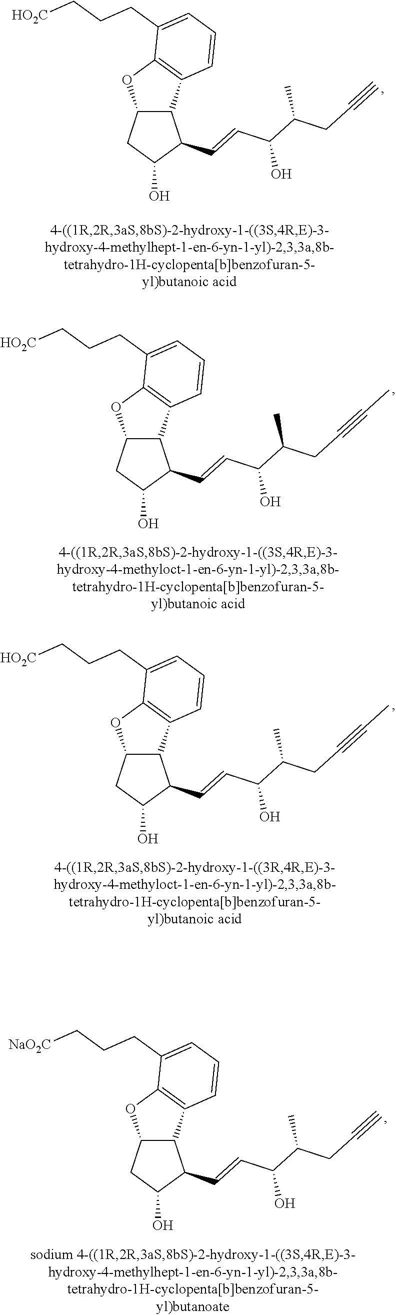

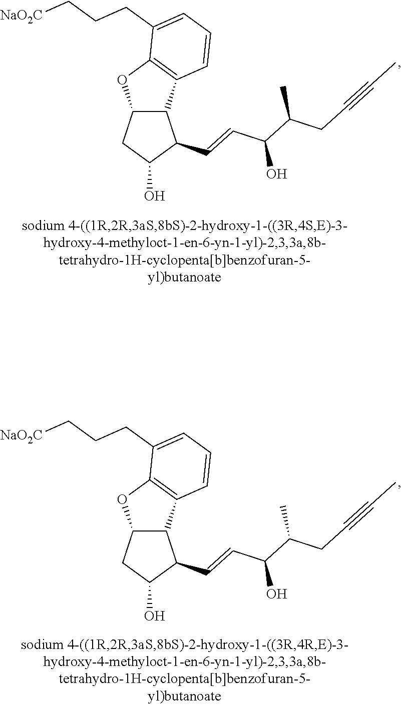

and a combination thereof.

Embodiment 32. A compound of the formula:

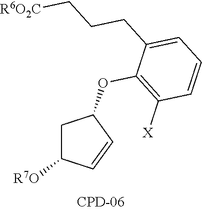

wherein
R⁶ is C₁₋₆ alkyl;
R⁷ is a hydroxy protecting group; and
X is halide.

Embodiment 33. The compound of embodiment 32, wherein the compound is:

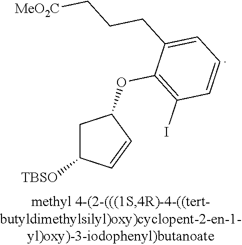 [SMILES: COC(=O)CCCc1cccc(I)c1O[C@@H]1C=C[C@H](O[Si](C)(C)C(C)(C)C)C1]

Embodiment 34. A composition comprising a compound of Formula I, or a pharmaceutically acceptable salt thereof, prepared by a process comprising the steps of any one of embodiments 1-31.

Embodiment 35. A method for preparing a solid oral dosage form satisfying content uniformity of 95% to 105% of active ingredients comprising; an active agent containing an effective amount of BPS-314d or a pharmaceutically acceptable salt thereof; and a solid oral dosage form prepared by wet granulation containing pharmaceutically acceptable additives.

Embodiment 36. The method of embodiment 35, wherein the active agent is present in an amount of 0.5% wt % or less based on the total weight of the solid oral dosage form.

Embodiment 37. The method of embodiment 35 or 36, wherein the active agent is present in an amount of 0.005 to 0.1% wt %.

Embodiment 38. The method of embodiment 35 or 37, wherein the solid oral dosage form is a tablet and the core of the tablet is a pharmaceutically acceptable additive, which includes one or more substances selected from excipients, disintegrants, binder, and lubricant.

Embodiment 39. The method of embodiment 38, wherein the excipient is at least one of lactose hydrate or microcrystalline cellulose.

Embodiment 40. The method of embodiment 38, wherein the disintegrant is at least one of starch, pregelatinized starch (starch 1500@).

Embodiment 41. The method of embodiment 38, wherein the binder includes one of either hyroxypropylcellulose or hydroxypropylmethylcellulose.

Embodiment 42. The method of embodiment 35 or 37, further comprising a coating base and wherein the coating base comprises hydroxypropylmethylcellulose.

Embodiment 43. A composition comprising a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0%.

Embodiment 44. The composition of embodiment 42, wherein the compound of Formula I is:

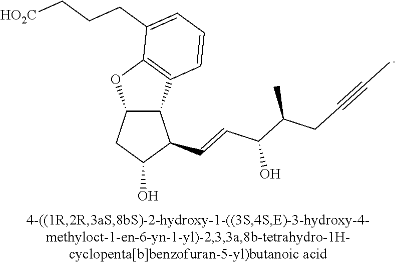 [SMILES: CC#CC[C@H](C)[C@H](O)/C=C/[C@@H]1[C@H]2c3cccc(CCCC(=O)O)c3O[C@H]2C[C@H]1O]

Embodiment 45. The composition of embodiment 42, wherein the pharmaceutically acceptable salt of a compound of Formula I is:

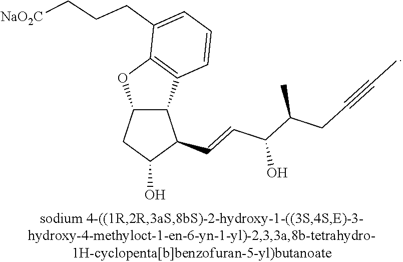

Embodiment 46. The composition of embodiment 43, wherein the composition does not contain more than about 1.0% of an isomer other than the compound of Formula I.

Embodiment 47. The composition of embodiment 43, wherein the composition does not contain more than about 0.15% of an isomer other than the compound of Formula I.

Embodiment 48. The composition of embodiment 43, wherein the composition does not contain more than about 0.1% of an isomer other than the compound of Formula I.

Embodiment 49. The composition of embodiment 43, wherein the composition does not contain more than about 0.05% of an isomer other than the compound of Formula I.

Embodiment 50. The composition of embodiment 43, wherein the compound of Formula I is at least about 95% pure.

Embodiment 51. The composition of embodiment 43, wherein the compound of Formula I is at least about 99% pure.

Embodiment 52. The composition of embodiment 43, wherein the compound of Formula I is at least about 99.8% pure.

Embodiment 53. The composition of embodiment 43, wherein any individual impurity is present in an amount of less than about 0.15%.

Embodiment 54. The composition of embodiment 43, wherein any individual impurity is present in an amount of less than about 0.1%.

Embodiment 55. The composition of embodiment 43, wherein any individual impurity is present in an amount of less than about 0.05%.

Embodiment 56. The composition of embodiment 43, wherein the total amount of the impurities is in an amount of less than about 10.0%.

Embodiment 57. The composition of embodiment 43, wherein the total amount of the impurities is in an amount of less than about 5.0%.

Embodiment 58. The composition of embodiment 43, wherein the total amount of the impurities is in an amount of less than about 1.0%.

Embodiment 59. The composition of embodiment 43, wherein the total amount of the impurities is in an amount of less than about 0.2%.

Embodiment 60. The composition of embodiment 43, wherein the impurity is selected from:

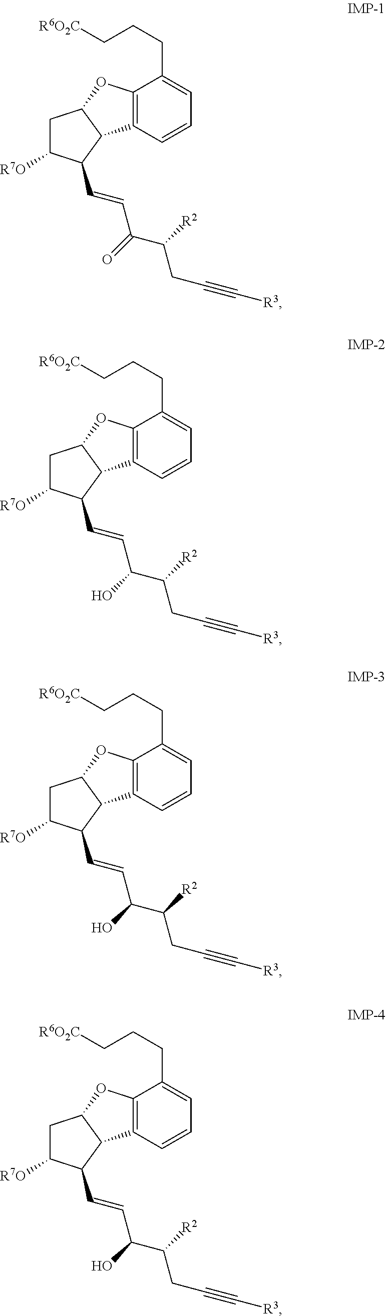

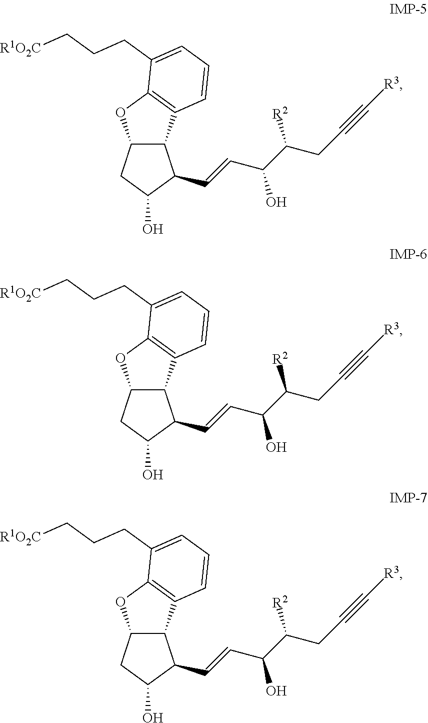

and a combination thereof.

Embodiment 61. The composition of embodiment 43, wherein the impurity is selected from:

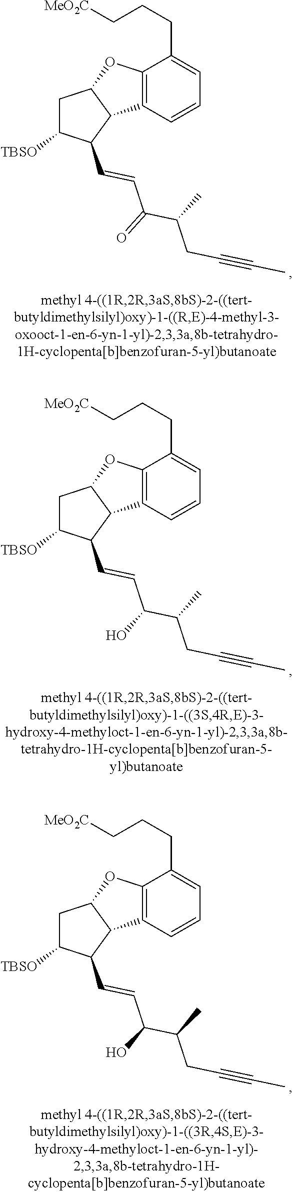 [SMILES: CC#CC[C@@H](C)C(=O)/C=C/[C@@H]1[C@H]2c3cccc(CCCC(=O)OC)c3O[C@H]2C[C@H]1O[Si](C)(C)C(C)(C)C ; CC#CC[C@@H](C)[C@H](O)/C=C/[C@@H]1[C@H]2c3cccc(CCCC(=O)OC)c3O[C@H]2C[C@H]1O[Si](C)(C)C(C)(C)C ; CC#CC[C@H](C)[C@@H](O)/C=C/[C@@H]1[C@H]2c3cccc(CCCC(=O)OC)c3O[C@H]2C[C@H]1O[Si](C)(C)C(C)(C)C]

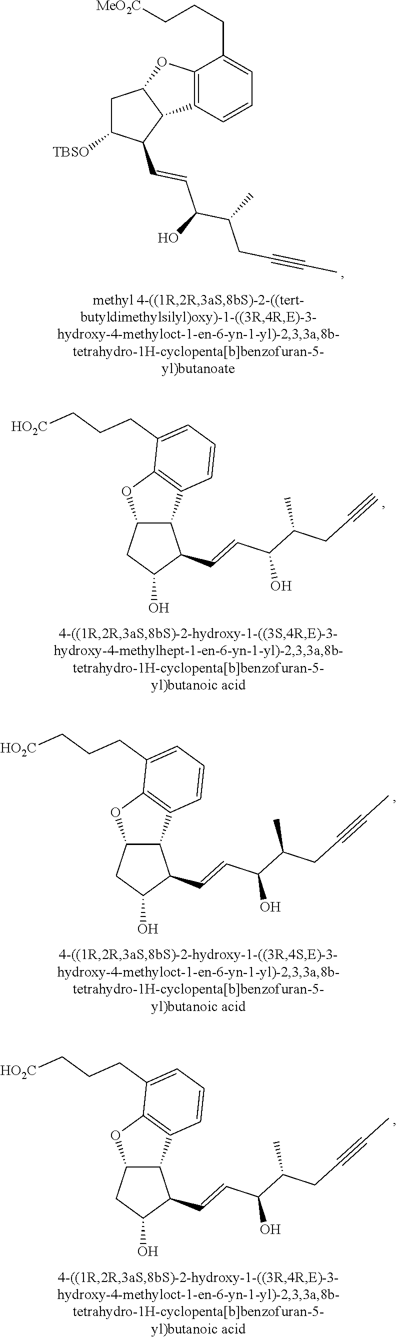 [SMILES: CC#CC[C@@H](C)[C@@H](O)/C=C/[C@@H]1[C@H]2c3cccc(CCCC(=O)OC)c3O[C@H]2C[C@H]1O[Si](C)(C)C(C)(C)C ; C#CC[C@@H](C)[C@H](O)/C=C/[C@@H]1[C@H]2c3cccc(CCCC(=O)O)c3O[C@H]2C[C@H]1O ; CC#CC[C@H](C)[C@@H](O)/C=C/[C@@H]1[C@H]2c3cccc(CCCC(=O)O)c3O[C@H]2C[C@H]1O ; CC#CC[C@@H](C)[C@@H](O)/C=C/[C@@H]1[C@H]2c3cccc(CCCC(=O)O)c3O[C@H]2C[C@H]1O]

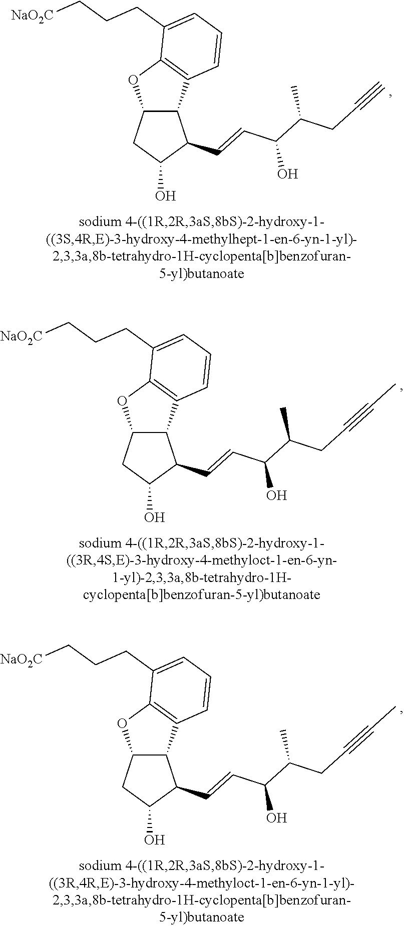

and a combination thereof.

Embodiment 62. The composition of embodiment 43, wherein the composition contains not more than about 0.5 wt % degradation product.

Embodiment 63. The composition of embodiment 43, wherein the composition contains not more than about 0.25 wt % degradation product.

Embodiment 64. The composition of embodiment 43, wherein the composition contains not more than about 0.1 wt % degradation product.

Embodiment 65. The composition of embodiment 43, formulated as a tablet, capsule, granule, powder, liquid, suspension, gel, syrup, slurry, suppository, patch, nasal spray, aerosol, injectable, implantable sustained-release formulation, or mucoadherent film.

Embodiment 66. The composition of embodiment 43, formulated as a tablet, a bi-layer tablet, a capsule, a multiparticulate, a drug coated sphere, a matrix tablet, or a multicore tablet.

Embodiment 67. The composition of embodiment 43, formulated as a tablet.

Embodiment 68. The composition of embodiment 43, formulated as a capsule.

Embodiment 69. The composition of embodiment 43, wherein the composition comprises about 1 mg to about 500 mg of a compound of Formula I.

Embodiment 70. The composition of embodiment 43, wherein the composition comprises about 20 mg of a compound of Formula I.

Embodiment 71. The composition of embodiment 43, wherein the composition comprises about 10 mg of a compound of Formula I.

Embodiment 72. The composition of embodiment 43, wherein the composition comprises about 5 mg of a compound of Formula I.

Embodiment 73. A method of treating cytokine release syndrome (CRS) in a subject, the method comprising administering to the subject a composition comprising an effective amount of the compound of Formula I or a pharmaceutically acceptable salt thereof according to any one of embodiments 32 to 72; wherein the CRS is treated in the subject.

Embodiment 74. The method of embodiment 73, wherein the compound of Formula I is:

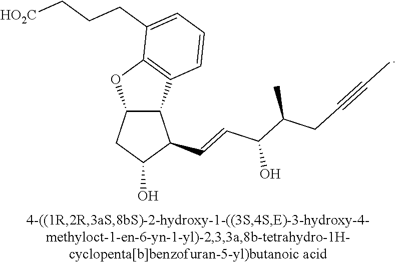 [SMILES: CC#CC[C@H](C)[C@H](O)/C=C/[C@@H]1[C@H]2c3cccc(CCCC(=O)O)c3O[C@H]2C[C@H]1O]

Embodiment 75. The method of embodiment 73, wherein the pharmaceutically acceptable salt of a compound of Formula I is:

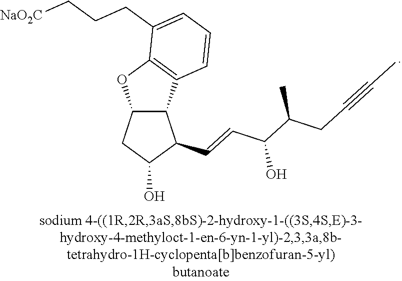

Embodiment 76. The method of any one of embodiments 73 to 75, wherein the composition does not contain more than about 1.0% of an isomer other than the compound of Formula I.

Embodiment 77. The method of any one of embodiments 73 to 76, wherein the composition does not contain more than about 0.15% of an isomer other than the compound of Formula I.

Embodiment 78. The method of any one of embodiments 73 to 77, wherein the composition does not contain more than about 0.1% of an isomer other than the compound of Formula I.

Embodiment 79. The method of any one of embodiments 73 to 78, wherein the composition does not contain more than about 0.05% of an isomer other than the compound of Formula I.

Embodiment 80. The method of any one of embodiments 73 to 79, wherein the compound of Formula I is at least about 95% pure.

Embodiment 81. The method of any one of embodiments 73 to 80, wherein the compound of Formula I is at least about 99% pure.

Embodiment 82. The method of any one of embodiments 73 to 81, wherein the compound of Formula I is at least about 99.8% pure.

Embodiment 83. The method of any one of embodiments 73 to 82, wherein any individual impurity is present in an amount of less than about 0.15%.

Embodiment 84. The method of any one of embodiments 73 to 83, wherein any individual impurity is present in an amount of less than about 0.1%.

Embodiment 85. The method of any one of embodiments 73 to 84, wherein any individual impurity is present in an amount of less than about 0.05%.

Embodiment 86. The method of any one of embodiments 73 to 85, wherein the composition contains not more than about 0.5 wt % degradation product.

Embodiment 87. The method of any one of embodiments 73 to 86, wherein the composition contains not more than about 0.25 wt % degradation product.

Embodiment 88. The method of any one of embodiments 73 to 87, wherein the composition contains not more than about 0.1 wt % degradation product.

Embodiment 89. The method of any one of embodiments 73 to 90, wherein the compound of Formula I or pharmaceutically acceptable salt thereof is present in an effective amount of the composition, wherein the effective amount is about 1 microgram to about 100 micrograms.

Embodiment 90. The method of any one of embodiments 73 to 89, wherein the effective amount of the compound of Formula I or a pharmaceutically acceptable salt thereof is about 0.1 microgram to about 5000 micrograms.

Embodiment 91. The method of any one of embodiments 73 to 90, wherein the effective amount of the compound of Formula I or a pharmaceutically acceptable salt thereof is about 15 micrograms to about 90 micrograms.

Embodiment 92. The method of any one of embodiments 73 to 91, wherein the effective amount of the compound of Formula I or a pharmaceutically acceptable salt thereof is about 60 micrograms to about 360 micrograms.

Embodiment 93. The method of any one of embodiments 73 to 92, wherein the composition is administered once per day.

Embodiment 94. The method of any one of embodiments 73 to 93, wherein the composition is administered twice per day.

Embodiment 95. The method of any one of embodiments 73 to 93, wherein the composition is administered three times per day.

Embodiment 96. The method of embodiment 90, wherein the effective amount of the compound of Formula I or a pharmaceutical salt thereof is 10 micrograms and wherein the effective amount is administered three times per day.

Embodiment 97. The method of embodiment 90, wherein the effective amount of the compound of Formula I or a pharmaceutical salt thereof is 20 micrograms and wherein the effective amount is administered three times per day.

Embodiment 98. The method of embodiment 90, wherein the effective amount of the compound of Formula I or a pharmaceutical salt thereof is 20 micrograms and wherein the effective amount is administered three times per day.

Embodiment 99. The method of any one of embodiments 73 to 93, wherein the composition is administered 4 times per day.

Embodiment 100. The method of any one of embodiments 73 to 99, wherein the composition is administered for a period of about 1 day to about 30 days.

Embodiment 101. The method of any one of embodiments 73 to 99, wherein the composition is administered for a period of about 7 days to about 14 days.

Embodiment 102. The method of any one of embodiments 73 to 101, further comprising administering a second composition comprising at least one corticosteroid, tocilizumab, IL-6 receptor blocker, or combinations thereof.

Embodiment 103. The method of embodiment 102, wherein the composition and the second composition are administered concurrently.

Embodiment 104. The method of any one of embodiments 102 to 103, wherein the subject requires reduced treatment with the second composition relative to a subject who does not receive the composition comprising Formula I.

Embodiment 105. The method of any one of embodiments 73 to 104, wherein the composition further comprises at least one excipient selected from at least one filler, at least one disintegrant, at least one binder, at least one wetting agent, at least one lubricant, at least one glidant, at least one preservative agent, at least one flavoring agent, at least one antioxidant, and combinations thereof.

Embodiment 106. The method of any one of embodiments 73 to 105, wherein the composition is in a formulation selected from as a tablet, a capsule, a granule, a powder, a liquid, a suspension, a gel, a syrup, a slurry, a suppository, a patch, a nasal spray, an aerosol, an injectable, an implantable sustained-release formulation, and a mucoadherent film.

Embodiment 107. The method of any one of embodiments 73 to 106, wherein the administration of the composition is selected from topical delivery, subcutaneous delivery, intravenous injection (IV) delivery, intramuscular injection (IM) delivery, intrathecal injection (IT) delivery, intraperitoneal injection (IP) delivery, transdermal delivery, subcutaneous delivery, oral delivery, transmucosal oral delivery, pulmonary delivery, inhalation delivery, intranasal delivery, buccal delivery, rectal delivery, vaginal delivery, and combinations thereof.

Embodiment 108. The method of any one of embodiments 73 to 107, wherein the administering comprises oral delivery or intravenous injection (IV) delivery.

Embodiment 109. The method of any one of embodiments 73 to 108, wherein the subject is receiving Chimeric Antigen Receptor (CAR) T-cells to treat cancer; and the cancer is selected from B-cell lymphoma, aggressive, relapsed or refractory diffuse large B cell lymphoma, primary mediastinal B-cell lymphoma, high grade B-cell lymphoma, transformed follicular lymphoma, relapsed or refractory mantle cell lymphoma, acute lymphoblastic leukemia, Hodgkin lymphoma, non-Hodgkin lymphoma, acute myeloid leukemia, and multiple myeloma.

Embodiment 110. The method of any one of embodiments 73 to 108, wherein the subject is receiving CAR T-cells to treat cancer; and the cancer is selected from brain cancer, breast cancer, glioblastoma, lung cancer, non-small-cell lung cancer, multiple myeloma, ovarian cancer, neuroblastoma, colorectal, biliary, pancreatic, mesothelioma, hepatoblastoma, embryonal sarcoma, prostate, sarcoma, and liver metastases.

Embodiment 111. The method of any one of embodiments 73 to 108, wherein the subject is receiving CAR T-cells to treat cancer; and the cancer is B-cell lymphoma.

Embodiment 112. The method of any one of embodiments 109 to 111, wherein the CAR T-cells are autologous CAR T-cells.

Embodiment 113. The method of any one of embodiments 109 to 111, wherein the CAR T-cells are allogeneic CAR T-cells.

Embodiment 114. The method of any one of embodiments 109 to 113, wherein the composition does not reduce a cell killing mediated by the CAR T cells by more than about 5% compared to cell killing without the composition.

Embodiment 115. The method of any one of embodiments 109 to 114, wherein the CAR T-cells are selected from BCMA CAR-T cells, CD19 CAR-T cells, CD19-CD3 bispecific CAR-T cells, and combinations thereof.

Embodiment 116. The method of any one of embodiments 109 to 115, wherein the composition is administered to the subject before the CAR T-cells are administered to the subject.

Embodiment 117. The method of any one of embodiments 109 to 116, wherein the second composition is administered to the subject after the CAR T-cells are administered to the subject.

Embodiment 118. The method of any one of embodiments 109 to 117, wherein the composition and the second composition are administered to the subject after the CAR T-cells are administered to the subject.

Embodiment 119. The method of any one of embodiments 109 to 118, wherein the composition and the second composition are administered to the subject before the CAR T-cells are administered to the subject.

Embodiment 120. The method of any one of embodiments 109 to 119, wherein the composition is administered to the subject starting about 1 day, about 2 days, about 3 days, about 4 days, about 5 days, about 6 days, or about 7 days after the CAR T-cells are administered to the subject.

Embodiment 121. The method of any one of embodiments 1099 to 119, wherein the composition is administered to the subject starting about 3 days to about 7 days after the CAR T-cells are administered to the subject.

Embodiment 122. The method of any one of embodiments 109 to 119, wherein the composition is administered starting one day before administration of the CAR T-cells and continued for a period of at least about 14 days.

Embodiment 123. The method of any one of embodiments 73 to 108, wherein the subject is receiving at least one bispecific antibody to treat cancer; and the cancer is selected from B-cell lymphoma, aggressive, relapsed or refractory diffuse large B cell lymphoma, primary mediastinal B-cell lymphoma, high grade B-cell lymphoma, transformed follicular lymphoma, relapsed or refractory mantle cell lymphoma, acute lymphoblastic leukemia, Hodgkin lymphoma, non-Hodgkin lymphoma, acute myeloid leukemia, and multiple myeloma.

Embodiment 124. The method of any one of embodiments 73 to 108, wherein the subject is receiving at least one bispecific antibody to treat cancer; and the cancer is selected from brain cancer, breast cancer, glioblastoma, lung cancer, non-small-cell lung cancer, multiple myeloma, ovarian cancer, neuroblastoma, colorectal, biliary, pancreatic, mesothelioma, hepatoblastoma, embryonal sarcoma, prostate, sarcoma, and liver metastases.

Embodiment 125. The method of any one of embodiments 73 to 108, wherein the subject is receiving at least one bispecific antibody to treat cancer; and the cancer is B-cell lymphoma.

Embodiment 126. The method of any one of embodiments 123 to 125 wherein the at least one bispecific antibody has a mechanism of action selected from recruitment and activation of immune cells, blocking of immune checkpoints, blocking of inflammatory factors, blocking of dual signal pathways, and combinations thereof.

Embodiment 127. The method of any one of embodiments 123 to 126, wherein the at least one bispecific antibody is selected from Blincyto (Blinatumomab), Hemlibra (emicizumab-kxwh), Rybrevant (amivantamab-vmjw), Kimmtrak (tebentafusp-tebn), and combinations thereof.

Embodiment 128. The method of any one of embodiments 123 to 126, wherein the at least one bispecific antibody is selected from a CD19-CD3 bispecific antibody, a Factor IX-Factor X bispecific antibody, an EGFR-MET bispecific antibody, a gp100-CD3 bispecific antibody, a BCMA-CD3 bispecific antibody, a CD20-CD3 bispecific antibody, a GPRC5D-CD3 bispecific antibody, and combinations thereof.

Embodiment 129. The method of any one of embodiments 123 to 128, wherein the composition is administered to the subject before the at least one bispecific antibody is administered to the subject.

Embodiment 130. The method of any one of embodiments 123 to 129, wherein the second composition is administered to the subject after the at least one bispecific antibody is administered to the subject.

Embodiment 131. The method of any one of embodiments 123 to 103, wherein the composition and the second composition are administered to the subject after the at least one bispecific antibody is administered to the subject.

Embodiment 132. The method of any one of embodiments 123 to 131, wherein the composition and the second composition are administered to the subject before the at least one bispecific antibody is administered to the subject.

Embodiment 133. The method of any one of embodiments 123 to 132, wherein the composition is administered to the subject starting about 1 day, about 2 days, about 3 days, about 4 days, about 5 days, about 6 days, or about 7 days after the at least one bispecific antibody is administered to the subject.

Embodiment 134. The method of any one of embodiments 123 to 132, wherein the composition is administered to the subject starting about 3 days to about 7 days after the at least one bispecific antibody is administered to the subject.

Embodiment 135. The method of any one of embodiments 123 to 132, wherein the composition is administered starting one day before administration of the at least one bispecific antibody and continued for a period of at least about 14 days.

Embodiment 136. The method of any one of embodiments 73 to 135, wherein the CRS is a grade 1 CRS, a grade 2 CRS, a grade 3 CRS, or a grade 4 CRS.

Embodiment 137. The method of any one of embodiments 73 to 136, wherein the subject experiences reduced severity measurements associated with CRS upon receiving the composition relative to subject who does not receive the composition.

Embodiment 138. The method of any one of embodiments 73 to 137, wherein treatment results in a decrease in an incidence of adverse events relative to a subject who is not administered the composition.

Embodiment 139. The method of any one of embodiments 73 to 138, wherein treatment of CRS with the composition is selected from a reduction in an incidence of hospitalizations, reduction in the use of anticytokine therapies other than the composition, reduction in a concentration of at least one proinflammatory cytokine in a blood sample, and combinations thereof relative to a subject who is not administered the composition.

Embodiment 140. The method of any one of embodiments 73 to 139, wherein the subject experiences reduced Parkinsonism effects upon receiving the composition relative to a subject who does not receive the composition.

Embodiment 141. The method of any one of embodiments 73 to 140, wherein treatment of CRS with the composition reduces levels of one or more of cytokines selected from IL-1α, IL-1β, IL-2, IL-4, IL-6, IL-7, IL-8, IL-10, IL-12, IFN-γ, TNF-α, IP-10, MCP-1, MIP-1, RANTES, GM-CSF, and combinations thereof in the subject relative to a subject who is not administered the composition.

Embodiment 142. The method of any one of embodiments 73 to 141, wherein the composition is administered once onset of CRS is detected by an increased level of one or more cytokines selected from IL-6, IL-10, IFN-γ, TNF-α, MIF, IL-5, IL-17A, IL-23, CXCL9/MIG, GCSF, VEGF-A, TGF-β, one or more of inflammatory biomarker C-reactive protein (CRP) and ferritin, and combinations thereof.

Embodiment 143. The method of any one of embodiments 73 to 142, wherein the subject is 18 years or older.

Embodiment 144. The method of any one of embodiments 73 to 143, wherein the subject has received leukapheresis.

Embodiment 145. The method of any one of embodiments 73 to 144, wherein the subject has adequate organ function, wherein adequate organ function is: Estimated Creatinine Clearance per Cockroft Gault formula in an amount of greater than or equal to about 60 mL/min; serum alanine aminotransferase/aspartate aminotransferase in an amount of less than or equal to about 2.5×upper limit normal (ULN); total bilirubin in an amount of less than or equal to about 1.5×ULN; left ventricular ejection fraction ≥40% on echocardiogram (ECG) or multigated acquisition and no clinically significant pericardial effusion; platelets in an amount of greater than or equal to about 50,000/mm3; absolute neutrophil count in an amount of greater than about 1000/μL; and Absolute lymphocyte count in an amount of greater than about 100/μL.

Embodiment 146. The method of any one of embodiments 73 to 145, wherein the subject has Eastern Cooperative Group performance status of 0 to 1.

Embodiment 147. The method of any one of embodiments 73 to 146, wherein the subject does not receive chemotherapy within about 14 days prior to leukapheresis.

Embodiment 148. The method of any one of embodiments 73 to 147, wherein the subject does not have Grade 2 or greater electrolyte imbalance, selected from potassium at less than about 3.0 mmol/L or greater than about 5.5 mmol/L; sodium at less than about 130 mmol/L or greater than about 150 mmol/L; calcium at less than about 8.0 mg/dL or greater than about 11.5 mg/dL; magnesium at less than about 0.5 mmol/L or greater than about 1.23 mmol/L, and combinations thereof.

Embodiment 149. The method of any one of embodiments 73 to 148, wherein the subject does not have a significant ECG abnormality, selected from a corrected QT interval (QTcF) value of greater than about 470 msec, conduction block disorder, atrial arrythmia, ventricular arrythmia, and combinations thereof.

Embodiment 150. The method of any one of embodiments 73 to 149, wherein the subject does not have any clinically significant cardiovascular disease selected from cerebral vascular accident, cerebral vascular stroke, myocardial infarction, unstable angina, congestive heart failure greater than or equal to New York Heart Associate Classification Class III, and combinations thereof.

Embodiment 151. The method of any one of embodiments 73 to 150, wherein the subject has not had an uncontrolled thromboembolic event, recent severe hemorrhage within about 6 months, and combinations thereof.

Embodiment 152. The method of any one of embodiments 73 to 151, wherein the subject does not have a known history of a bleeding disorder.

Embodiment 153. The method of any one of embodiments 73 to 152, wherein the subject has a baseline systolic blood pressure of greater than about 100 mmHg.

Embodiment 154. The method of any one of embodiments 73 to 153, wherein the subject does not have a history of autoimmune disease, graft versus host disease, or combinations thereof.

Embodiment 155. The method of embodiment 154, wherein the subject has not received immunosuppressive therapy other than steroids at a dose of about 5 mg or less within about 2 years.

Embodiment 156. A method of treating immune effector cell-associated neurotoxicity syndrome (ICANS) in a subject, the method comprising administering to the subject a composition comprising an effective amount of the compound of Formula I or a pharmaceutically acceptable salt thereof according to any one of embodiments 34 to 73; wherein the ICANS is treated.

Embodiment 157. The method of embodiment 156, wherein the compound of Formula I is:

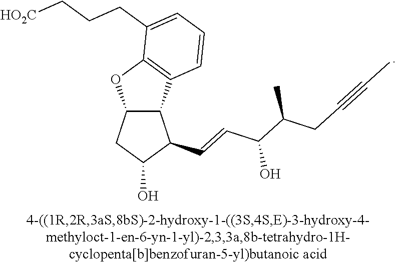 [SMILES: CC#CC[C@H](C)[C@H](O)/C=C/[C@@H]1[C@H]2c3cccc(CCCC(=O)O)c3O[C@H]2C[C@H]1O]

Embodiment 158. The method of embodiment 156, wherein the pharmaceutically acceptable salt of a compound of Formula I is:

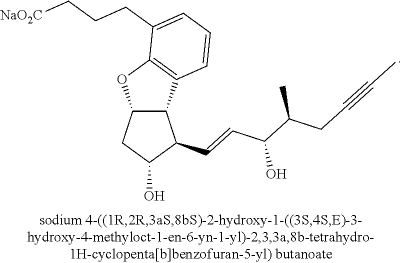

Embodiment 159. The method of any one of embodiments 156 to 158, wherein the composition does not contain more than about 1.0% of an isomer other than the compound of Formula I.

Embodiment 160. The method of any one of embodiments 156 to 159, wherein the composition does not contain more than about 0.15% of an isomer other than the compound of Formula I.

Embodiment 161. The method of any one of embodiments 156 to 160, wherein the composition does not contain more than about 0.1% of an isomer other than the compound of Formula I.

Embodiment 162. The method of any one of embodiments 156 to 161, wherein the composition does not contain more than about 0.05% of an isomer other than the compound of Formula I.

Embodiment 163. The method of any one of embodiments 156 to 162, wherein the compound of Formula I is at least about 95% pure.

Embodiment 164. The method of any one of embodiments 156 to 163, wherein the compound of Formula I is at least about 99% pure.

Embodiment 165. The method of any one of embodiments 156 to 164, wherein the compound of Formula I is at least about 99.8% pure.

Embodiment 166. The method of any one of embodiments 156 to 165, wherein any individual impurity is present in an amount of less than about 0.15%.

Embodiment 167. The method of any one of embodiments 156 to 166, wherein any individual impurity is present in an amount of less than about 0.1%.

Embodiment 168. The method of any one of embodiments 156 to 167, wherein any individual impurity is present in an amount of less than about 0.05%.

Embodiment 169. The method of any one of embodiments 156 to 168, wherein the composition contains not more than about 0.5 wt % degradation product.

Embodiment 170. The method of any one of embodiments 156 to 169, wherein the composition contains not more than about 0.25 wt % degradation product.

Embodiment 171. The method of any one of embodiments 156 to 170, wherein the composition contains not more than about 0.1 wt % degradation product.

Embodiment 172. The method of any one of embodiments 156 to 171, wherein the compound of Formula I or pharmaceutically acceptable salt thereof is present in a unit dose of the composition in an amount of about 1 microgram to about 100 micrograms.

Embodiment 173. The method of any one of embodiments 156 to 172, wherein the effective amount of the compound of Formula I or a pharmaceutically acceptable salt thereof is at least about 0.1 microgram.

Embodiment 174. The method of any one of embodiments 156 to 173, wherein the effective amount of the compound of Formula I or a pharmaceutically acceptable salt thereof is about 0.1 microgram to about 5000 micrograms.

Embodiment 175. The method of any one of embodiments 156 to 174, wherein the effective amount of the compound of Formula I or a pharmaceutically acceptable salt thereof is about 15 micrograms to about 90 micrograms.

Embodiment 176. The method of any one of embodiments 156 to 175, wherein the effective amount of the compound of Formula I or a pharmaceutically acceptable salt thereof is about 60 micrograms to about 360 micrograms.

Embodiment 177. The method of any one of embodiments 156 to 176, wherein the composition is administered once per day.

Embodiment 178. The method of any one of embodiments 156 to 175, wherein the composition is administered twice per day.

Embodiment 179. The method of any one of embodiments 156 to 179, wherein the composition is administered three times per day.

Embodiment 180. The method of embodiment 179, wherein the effective amount of the compound of Formula I or a pharmaceutical salt thereof is 10 micrograms and wherein the effective amount is administered three times per day.

Embodiment 181. The method of embodiment 179, wherein the effective amount of the compound of Formula I or a pharmaceutical salt thereof is 20 micrograms and wherein the effective amount is administered three times per day.

Embodiment 182. The method of embodiment 179, wherein the effective amount of the compound of Formula I or a pharmaceutical salt thereof is 20 micrograms and wherein the effective amount is administered three times per day.

Embodiment 183. The method of any one of embodiments 156 to 177, wherein the composition is administered 4 times per day.

Embodiment 184. The method of any one of embodiments 156 to 183, wherein the composition is administered for a period of about 1 day to about 30 days.

Embodiment 185. The method of embodiment 184, wherein the composition is administered for 15 days.

Embodiment 186. The method of any one of embodiments 156 to 184, wherein the composition is administered for a period of about 7 days to about 14 days.

Embodiment 187. The method of any one of embodiments 156 to 186, further comprising administering a second composition comprising at least one corticosteroid, tocilizumab, IL-6 receptor blocker, or combinations thereof.

Embodiment 188. The method of embodiment 187, wherein the composition and the second composition are administered concurrently.

Embodiment 189. The method of any one of embodiments 187 to 188, wherein the subject requires reduced treatment with the second composition relative to a subject who does not receive the composition comprising Formula I.

Embodiment 190. The method of any one of embodiments 156 to 189, wherein the composition further comprises at least one excipient, at least one filler, at least one disintegrant, at least one binder, at least one wetting agent, at least one lubricant, at least one glidant, at least one preservative agent, at least one flavoring agent, at least one antioxidant, or combinations thereof.

Embodiment 191. The method of any one of embodiments 156 to 190, wherein the composition is in a formulation selected from as a tablet, a capsule, a granule, a powder, a liquid, a suspension, a gel, a syrup, a slurry, a suppository, a patch, a nasal spray, an aerosol, an injectable, an implantable sustained-release formulation, and a mucoadherent film.

Embodiment 192. The method of any one of embodiments 156 to 191, wherein the administration of the composition is selected from topical delivery, subcutaneous delivery, intravenous injection (IV) delivery, intramuscular injection (IM) delivery, intrathecal injection (IT) delivery, intraperitoneal injection (IP) delivery, transdermal delivery, subcutaneous delivery, oral delivery, transmucosal oral delivery, pulmonary delivery, inhalation delivery, intranasal delivery, buccal delivery, rectal delivery, vaginal delivery, and combinations thereof.

Embodiment 193. The method of any one of embodiments 156 to 192, wherein the administering comprises oral delivery or intravenous injection (IV) delivery.

Embodiment 194. The method of any one of embodiments 156 to 193, wherein the subject is receiving Chimeric Antigen Receptor (CAR) T-cells to treat cancer; and the cancer is selected from B-cell lymphoma, aggressive, relapsed or refractory diffuse large B cell lymphoma, primary mediastinal B-cell lymphoma, high grade B-cell lymphoma, transformed follicular lymphoma, relapsed or refractory mantle cell lymphoma, acute lymphoblastic leukemia, Hodgkin lymphoma, non-Hodgkin lymphoma, acute myeloid leukemia, and multiple myeloma.

Embodiment 195. The method of any one of embodiments 156 to 194, wherein the subject is receiving CAR T-cells to treat cancer; and the cancer is selected from brain cancer, breast cancer, glioblastoma, lung cancer, non-small-cell lung cancer, multiple myeloma, ovarian cancer, neuroblastoma, colorectal, biliary, pancreatic, mesothelioma, hepatoblastoma, embryonal sarcoma, prostate, sarcoma, and liver metastases.

Embodiment 196. The method of any one of embodiments 156 to 195, wherein the subject is receiving CAR T-cells to treat cancer; and the cancer is B-cell lymphoma.

Embodiment 197. The method of any one of embodiments 194 to 196, wherein the CAR T-cells are autologous CAR T-cells.

Embodiment 198. The method of any one of embodiments 194 to 196, wherein the CAR T-cells are allogeneic CAR T-cells.

Embodiment 199. The method of any one of embodiments 194 to 198, wherein the composition does not reduce a cell killing mediated by the CAR T cells by more than about 5% compared to cell killing without the composition.

Embodiment 200. The method of any one of embodiments 194 to 199, wherein the CAR T-cells are selected from BCMA CAR-T cells, CD19 CAR-T cells, CD19-CD3 bispecific CAR-T cells, and combinations thereof.

Embodiment 201. The method of any one of embodiments 194 to 200, wherein the composition is administered to the subject before the CAR T-cells are administered to the subject.

Embodiment 202. The method of any one of embodiments 194 to 201, wherein the second composition is administered to the subject after the CAR T-cells are administered to the subject.

Embodiment 203. The method of any one of embodiments 194 to 202, wherein the composition and the second composition are administered to the subject after the CAR T-cells are administered to the subject.

Embodiment 204. The method of any one of embodiments 194 to 203, wherein the composition and the second composition are administered to the subject before the CAR T-cells are administered to the subject.

Embodiment 205. The method of any one of embodiments 194 to 204, wherein the composition is administered to the subject starting about 1 day, about 2 days, about 3 days, about 4 days, about 5 days, about 6 days, or about 7 days after the CAR T-cells are administered to the subject.

Embodiment 206. The method of any one of embodiments 194 to 205, wherein the composition is administered to the subject starting about 3 days to about 7 days after the CAR T-cells are administered to the subject.

Embodiment 207. The method of any one of embodiments 194 to 206, wherein the composition is administered starting one day before administration of the CAR T-cells and continued for a period of at least about 14 days.

Embodiment 208. The method of any one of embodiments 156 to 207, wherein the subject is receiving at least one bispecific antibody to treat cancer; and the cancer is selected from B-cell lymphoma, aggressive, relapsed or refractory diffuse large B cell lymphoma, primary mediastinal B-cell lymphoma, high grade B-cell lymphoma, transformed follicular lymphoma, relapsed or refractory mantle cell lymphoma, acute lymphoblastic leukemia, Hodgkin lymphoma, non-Hodgkin lymphoma, acute myeloid leukemia, and multiple myeloma.

Embodiment 209. The method of any one of embodiments 156 to 207, wherein the subject is receiving at least one bispecific antibody to treat cancer; and the cancer is selected from brain cancer, breast cancer, glioblastoma, lung cancer, non-small-cell lung cancer, multiple myeloma, ovarian cancer, neuroblastoma, colorectal, biliary, pancreatic, mesothelioma, hepatoblastoma, embryonal sarcoma, prostate, sarcoma, and liver metastases.

Embodiment 210. The method of any one of embodiments 156 to 207, wherein the subject is receiving at least one bispecific antibody to treat cancer; and the cancer is B-cell lymphoma.

Embodiment 211. The method of any one of embodiments 208 to 210, wherein the at least one bispecific antibody has a mechanism of action selected from recruitment and activation of immune cells, blocking of immune checkpoints, blocking of inflammatory factors, blocking of dual signal pathways, and combinations thereof.

Embodiment 212. The method of any one of embodiments 208 to 211, wherein the at least one bispecific antibody is selected from Blincyto (Blinatumomab), Hemlibra (emicizumab-kxwh), Rybrevant (amivantamab-vmjw), Kimmtrak (tebentafusp-tebn), and combinations thereof.

Embodiment 213. The method of any one of embodiments 208 to 212, wherein the at least one bispecific antibody is selected from a CD19-CD3 bispecific antibody, a Factor IX-Factor X bispecific antibody, an EGFR-MET bispecific antibody, a gp100-CD3 bispecific antibody, a BCMA-CD3 bispecific antibody, a CD20-CD3 bispecific antibody, a GPRC5D-CD3 bispecific antibody, and combinations thereof.

Embodiment 214. The method of any one of embodiments 208 to 213, wherein the composition is administered to the subject before the at least one bispecific antibody is administered to the subject.

Embodiment 215. The method of any one of embodiments 208 to 214, wherein the second composition is administered to the subject after the at least one bispecific antibody is administered to the subject.

Embodiment 216. The method of any one of embodiments 208 to 215, wherein the composition and the second composition are administered to the subject after the at least one bispecific antibody is administered to the subject.

Embodiment 217. The method of any one of embodiments 208 to 216, wherein the composition and the second composition are administered to the subject before the at least one bispecific antibody is administered to the subject.

Embodiment 218. The method of any one of embodiments 208 to 217, wherein the composition is administered to the subject starting about 1 day, about 2 days, about 3 days, about 4 days, about 5 days, about 6 days, or about 7 days after the at least one bispecific antibody is administered to the subject.

Embodiment 219. The method of any one of embodiments 208 to 217, wherein the composition is administered to the subject starting about 3 days to about 7 days after the at least one bispecific antibody is administered to the subject.

Embodiment 220. The method of any one of embodiments 208 to 217, wherein the composition is administered starting one day before administration of the at least one bispecific antibody and continued for a period of at least about 14 days.

Embodiment 221. The method of any one of embodiments 156 to 220, wherein the ICANS is a grade 1 ICANS, a grade 2 ICANS, a grade 3 ICANS, or a grade 4 ICANS.

Embodiment 222. The method of any one of embodiments 156 to 221, wherein the subject experiences reduced severity measurements associated with ICANS upon receiving the composition relative to subject who does not receive the composition.

Embodiment 223. The method of any one of embodiments 156 to 222, wherein treatment results in a decrease in an incidence of adverse events relative to a subject who is not administered the composition.

Embodiment 224. The method of any one of embodiments 156 to 223, wherein treatment of ICANS with the composition is selected from a reduction in an incidence of hospitalizations, reduction in the use of anticytokine therapies other than the composition, reduction in a concentration of at least one proinflammatory cytokine in a blood sample, and combinations thereof relative to a subject who is not administered the composition.

Embodiment 225. The method of any one of embodiments 156 to 224, wherein the subject experiences reduced Parkinsonism effects upon receiving the composition relative to a subject who does not receive the composition.

Embodiment 226. The method of any one of embodiments 156 to 225, wherein treatment of ICANS with the composition reduces levels of one or more of cytokines selected from IL-1α, IL-1β, IL-2, IL-4, IL-6, IL-7, IL-8, IL-10, IL-12, IFN-γ, TNF-α, IP-10, MCP-1, MIP-1, RANTES, GM-CSF, and combinations thereof in the subject relative to a subject who is not administered the composition.

Embodiment 227. The method of any one of embodiments 156 to 226, wherein the composition is administered once onset of ICANS is detected by an increased level of one or more cytokines selected from IL-6, IL-10, IFN-7, TNF-α, MIF, IL-5, IL-17A, IL-23, CXCL9/MIG, GCSF, VEGF-A, TGF-β, one or more of inflammatory biomarker C-reactive protein (CRP) and ferritin, and combinations thereof.

Embodiment 228. The method of any one of embodiments 156 to 227, wherein the subject is 18 years or older.

Embodiment 229. The method of any one of embodiments 156 to 228, wherein the subject has received leukapheresis.

Embodiment 230. The method of any one of embodiments 156 to 229, wherein the subject has adequate organ function, wherein adequate organ function is: Estimated Creatinine Clearance per Cockroft Gault formula in an amount of greater than or equal to about 60 mL/min; serum alanine aminotransferase/aspartate aminotransferase in an amount of less than or equal to about 2.5×upper limit normal (ULN); total bilirubin in an amount of less than or equal to about 1.5×ULN; left ventricular ejection fraction ≥40% on echocardiogram (ECG) or multigated acquisition and no clinically significant pericardial effusion; platelets in an amount of greater than or equal to about 50,000/mm3; absolute neutrophil count in an amount of greater than about 1000/μL; and Absolute lymphocyte count in an amount of greater than about 100/μL.

Embodiment 231. The method of any one of embodiments 156 to 230, wherein the subject has Eastern Cooperative Group performance status of 0 to 1.

Embodiment 232. The method of any one of embodiments 156 to 231, wherein the subject does not receive cytotoxic chemotherapy within about 14 days prior to leukapheresis.

Embodiment 233. The method of any one of embodiments 156 to 232, wherein the subject does not have Grade 2 or greater electrolyte imbalance, including potassium at less than about 3.0 mmol/L or greater than about 5.5 mmol/L; sodium at less than about 130 mmol/L or greater than about 150 mmol/L; calcium at less than about 8.0 mg/dL or greater than about 11.5 mg/dL; magnesium at less than about 0.5 mmol/L or greater than about 1.23 mmol/L, and combinations thereof.

Embodiment 234. The method of any one of embodiments 156 to 233, wherein the subject does not have a significant ECG abnormality, selected from a corrected QT interval (QTcF) value of greater than about 470 msec, conduction block disorder, atrial arrythmia, ventricular arrythmia, and combinations thereof.

Embodiment 235. The method of any one of embodiments 156 to 234, wherein the subject does not have any clinically significant cardiovascular disease selected from cerebral vascular accident, cerebral vascular stroke, myocardial infarction, unstable angina, congestive heart failure greater than or equal to New York Heart Associate Classification Class III, and combinations thereof.

Embodiment 236. The method of any one of embodiments 156 to 235, wherein the subject has not had an uncontrolled thromboembolic event, recent severe hemorrhage within about 6 months, and combinations thereof.

Embodiment 237. The method of any one of embodiments 156 to 236, wherein the subject does not have a known history of a bleeding disorder.

Embodiment 238. The method of any one of embodiments 156 to 237, wherein the subject has a baseline systolic blood pressure of greater than about 100 mmHg.

Embodiment 239. The method of any one of embodiments 156 to 238, wherein the subject does not have a history of autoimmune disease, graft versus host disease, or combinations thereof.

Embodiment 240. The method of embodiment 239, wherein the subject has not received immunosuppressive therapy other than steroids at a dose of about 5 mg or less within about 2 years.

Embodiment 241. A method for preparing a compound of Formula I having the structure:

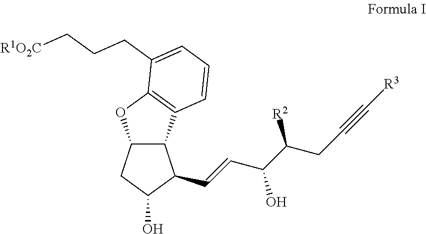

wherein
R¹ is selected from H and a cation;
R² and R³ are independently C₁₋₆ alkyl;
the method comprising the steps of:
(a) performing a condensation reaction on the compound

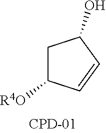

to form the compound

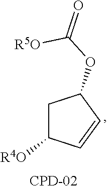

wherein R⁴ is C(O)C₁₋₆ alkyl and R⁵ is C₁₋₆ alkyl;
(b) coupling the carbonate of CPD-02 with

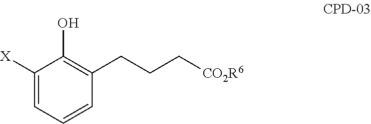

to form the compound

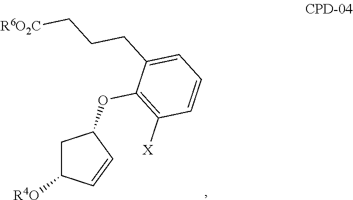

wherein is R⁶ is C₁₋₆ alkyl and X is halide;
(c) hydrolyzing CPD-04 to form the compound

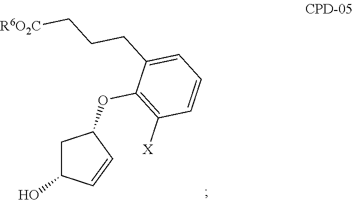

(d) protecting the alcohol of CPD-05 to form the compound

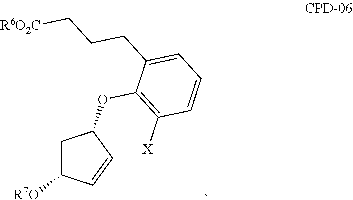

wherein R⁷ is a hydroxy protecting group;
(e) performing a radical cyclization and trapping reaction on CPD-06 with

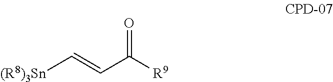

to form the compound

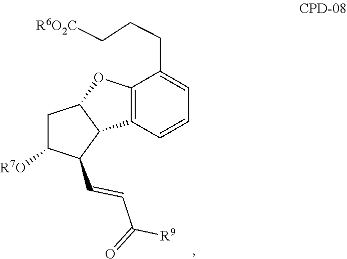

wherein R⁸ and R⁹ are independently C₁₋₆ alkyl; and
(f) converting CPD-08 to a compound of Formula I;
wherein the compound of Formula I is at least about 90% pure and wherein any individual impurity is present in an amount of less than about 1.0%.

Embodiment 242. The method of embodiment 241, further comprising cleaving the double bond of CPD-08 to from the compound

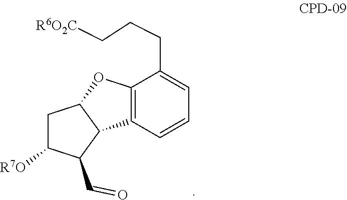

Embodiment 243. The method of embodiment 242, further comprising coupling CPD-09 with

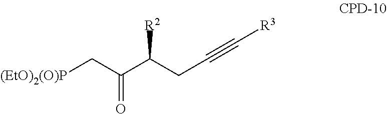

to form the compound

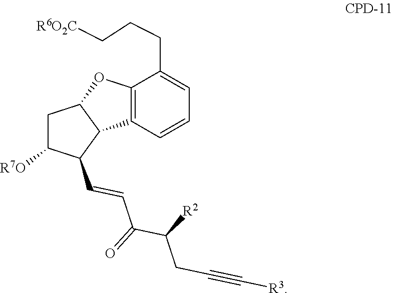

Embodiment 244. The method of embodiment 243, further comprising reduction of the ketone of CPD-11 to form the compound

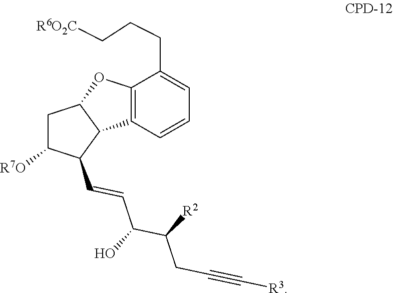

Embodiment 245. The method of embodiment 244, further comprising the deprotection of CPD-12 to form the compound

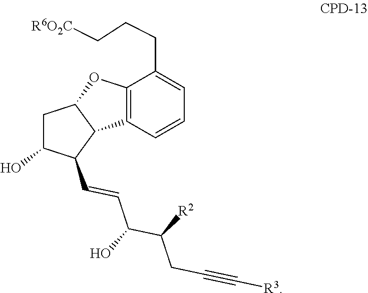

Embodiment 246. The method of embodiment 245, further comprising hydrolyzing CPD-13 to form the compound Formula I.

Embodiment 247. The method of embodiment 241, wherein R¹ is Na⁺.

Embodiment 248. The method of embodiment 241, wherein R², R³, and R⁶ are methyl.

Embodiment 249. The method of embodiment 241, wherein R⁴ is C(O)Me.

Embodiment 250. The method of embodiment 241, wherein R⁵ is ethyl.

Embodiment 251. The method of embodiment 241, wherein R⁷ is tert-butyldimethylsilyl.

Embodiment 252. The method of embodiment 241, wherein R⁸ is butyl.

Embodiment 253. The method of embodiment 241, wherein R⁹ is pentyl.

Embodiment 254. The method of embodiment 241, wherein the compound of Formula I is:

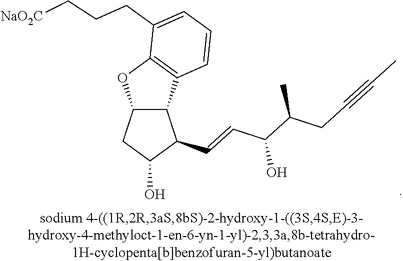

Embodiment 255. The method of embodiment 241, wherein the method does not produce more than about 1.0% of an isomer other than the compound of Formula I.

Embodiment 256. The method of embodiment 241, wherein the method does not produce more than about 0.15% of an isomer other than the compound of Formula I.

Embodiment 257. The method of embodiment 241, wherein the method does not produce more than about 0.1% of an isomer other than the compound of Formula I.

Embodiment 258. The method of embodiment 241, wherein the method does not produce more than about 0.05% of an isomer other than the compound of Formula I.

Embodiment 259. The method of embodiment 241, wherein the compound of Formula I is at least about 95% pure.

Embodiment 260. The method of embodiment 241, wherein the compound of Formula I is at least about 99% pure.

Embodiment 261. The method of embodiment 241, wherein the compound of Formula I is at least about 99.8% pure.

Embodiment 262. The method of embodiment 241, wherein any individual impurity is present in an amount of less than about 0.15%.

Embodiment 263. The method of embodiment 241, wherein any individual impurity is present in an amount of less than about 0.1%.

Embodiment 264. The method of embodiment 241, wherein any individual impurity is present in an amount of less than about 0.05%.

Embodiment 265. The method of embodiment 241, wherein the compound of Formula I is prepared in an overall yield of at least about 5%.

Embodiment 266. The method of embodiment 241, wherein the overall yield of the compound of Formula I is at least about 10%.

Embodiment 267. A compound of the formula:

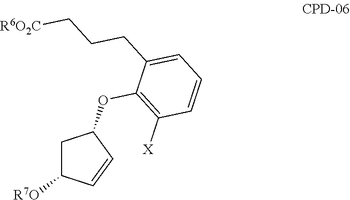

wherein
R⁶ is C₁₋₆ alkyl;
R⁷ is a hydroxy protecting group; and
X is halide.

Embodiment 268. The compound of embodiment 267, wherein the compound is:

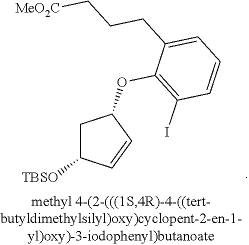 [SMILES: COC(=O)CCCc1cccc(I)c1O[C@@H]1C=C[C@H](O[Si](C)(C)C(C)(C)C)C1]

Embodiment 269. A method for preparing a solid oral dosage form satisfying content uniformity of 95% to 105% of active ingredients comprising; (a) an active agent containing an effective amount of BPS-314d or a pharmaceutically acceptable salt thereof; and (b) a solid oral dosage form prepared by wet granulation containing pharmaceutically acceptable additives.

Embodiment 270. The method of embodiment 269, wherein the active agent is present in an amount of 0.5% wt % or less based on the total weight of the solid oral dosage form.

Embodiment 271. The method according to embodiment 269 or 270, wherein the active agent is present in an amount of 0.005 to 0.1% wt %.

Embodiment 272. The method of embodiment 269 or 271, wherein the solid oral dosage form is a tablet and the core of the tablet is a pharmaceutically acceptable additive, which includes one or more substances selected from excipients, disintegrants, binder, and lubricant.

Embodiment 273. The method of embodiment 272, wherein the excipient is at least one of lactose hydrate or microcrystalline cellulose.

Embodiment 274. The method of embodiment 272, wherein the disintegrant is at least one of starch, pregelatinized starch (Starch 1500®).

Embodiment 275. The method of embodiment 272, wherein the binder includes one of either hyroxypropylcellulose or hydroxypropylmethylcellulose.

Embodiment 276. The method of embodiment 269 or 271, further comprising a coating base and wherein the coating base comprises hydroxypropylmethylcellulose.

Embodiment 277. A composition comprising a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0%.

Embodiment 278. The composition of embodiment 277, wherein the pharmaceutically acceptable salt of a compound of Formula I is:

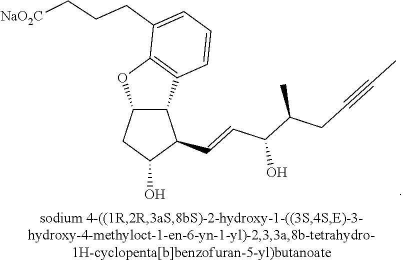

Embodiment 279. The composition of embodiment 277, wherein the composition does not contain more than about 1.0% of an isomer other than the compound of Formula I.

Embodiment 280. The composition of embodiment 277, wherein the composition does not contain more than about 0.15% of an isomer other than the compound of Formula I.

Embodiment 281. The composition of embodiment 277, wherein the composition does not contain more than about 0.1% of an isomer other than the compound of Formula I.

Embodiment 282. The composition of embodiment 277, wherein the composition does not contain more than about 0.05% of an isomer other than the compound of Formula I.

Embodiment 283. The composition of embodiment 277, wherein the compound of Formula I is at least about 95% pure.

Embodiment 284. The composition of embodiment 277, wherein the compound of Formula I is at least about 99% pure.

Embodiment 285. The composition of embodiment 277, wherein the compound of Formula I is at least about 99.8% pure.

Embodiment 286. The composition of embodiment 277, wherein any individual impurity is present in an amount of less than about 0.15%.

Embodiment 287. The composition of embodiment 277, wherein any individual impurity is present in an amount of less than about 0.1%.

Embodiment 288. The composition of embodiment 277, wherein any individual impurity is present in an amount of less than about 0.05%.

Embodiment 289. The composition of embodiment 277, wherein the composition contains not more than about 0.5 wt % degradation product.

Embodiment 290. The composition of embodiment 277, wherein the composition contains not more than about 0.25 wt % degradation product.

Embodiment 291. The composition of embodiment 277, wherein the composition contains not more than about 0.1 wt % degradation product.

Embodiment 292. The composition of embodiment 277, formulated as a tablet, capsule, granule, powder, liquid, suspension, gel, syrup, slurry, suppository, patch, nasal spray, aerosol, injectable, implantable sustained-release formulation, or mucoadherent film.

Embodiment 293. The composition of embodiment 277, formulated as a tablet, a bi-layer tablet, a capsule, a multiparticulate, a drug coated sphere, a matrix tablet, or a multicore tablet.

Embodiment 294. The composition of embodiment 277, formulated as a tablet.

Embodiment 295. The composition of embodiment 277, formulated as a capsule.

Embodiment 296. The composition of embodiment 277, wherein the composition comprises about 1 mg to about 500 mg of a compound of Formula I.

Embodiment 297. The composition of embodiment 277, wherein the composition comprises about 20 mg of a compound of Formula I.

Embodiment 298. The composition of embodiment 277, wherein the composition comprises about 10 mg of a compound of Formula I.

Embodiment 299. The composition of embodiment 277, wherein the composition comprises about 5 mg of a compound of Formula I.

Embodiment 300. A method of treating cancer in a subject, the method comprising administering at least one bispecific antibody and a first composition to the subject, wherein the first composition comprises at least an effective amount of a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0%.

Embodiment 301. The method of embodiment 300, wherein the first composition does not contain more than about 1.0% of an isomer other than the compound of Formula I.

Embodiment 302. The method of embodiment 300, wherein the first composition does not contain more than about 0.15% of an isomer other than the compound of Formula I.

Embodiment 303. The method of embodiment 300, wherein the first composition does not contain more than about 0.1% of an isomer other than the compound of Formula I.

Embodiment 304. The method of embodiment 300, wherein the first composition does not contain more than about 0.05% of an isomer other than the compound of Formula I.

Embodiment 305. The method of embodiment 300, wherein the compound of Formula I is at least about 95% pure.

Embodiment 306. The method of embodiment 300, wherein the compound of Formula I is at least about 99% pure.

Embodiment 307. The method of embodiment 300, wherein the compound of Formula I is at least about 99.8% pure.

Embodiment 308. The method of embodiment 300, wherein any individual impurity is present in an amount of less than about 0.15%.

Embodiment 309. The method of embodiment 300, wherein any individual impurity is present in an amount of less than about 0.1%.

Embodiment 310. The method of embodiment 300, wherein any individual impurity is present in an amount of less than about 0.05%.

Embodiment 311. The method of embodiment 300, wherein the first composition contains not more than about 0.5 wt % degradation product.

Embodiment 312. The method of embodiment 300, wherein the first composition contains not more than about 0.25 wt % degradation product.

Embodiment 313. The method of embodiment 300, wherein the first composition contains not more than about 0.1 wt % degradation product.

Embodiment 314. The method of embodiment 300, wherein the compound of Formula I is:

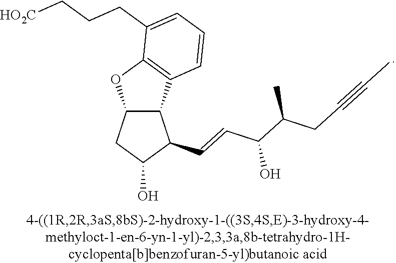 [SMILES: CC#CC[C@H](C)[C@H](O)/C=C/[C@@H]1[C@H]2c3cccc(CCCC(=O)O)c3O[C@H]2C[C@H]1O]

Embodiment 315. The method of embodiment 300, wherein the pharmaceutically acceptable salt of a compound of Formula I is:

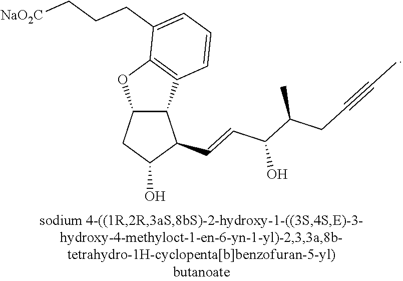

Embodiment 316. The method of embodiment 300, further comprising administering to the subject a second composition comprising at least one corticosteroid, tocilizumab, IL-6 receptor blocker, or combinations thereof.

Embodiment 317. The method of embodiment 300, further comprising administering to the subject a second composition comprising at least one corticosteroid, tocilizumab, IL-6 receptor blocker, or combinations thereof; wherein the subject requires reduced treatment with the second composition relative to a subject who does not receive the first composition.

Embodiment 318. The method of embodiment 300, wherein the first composition reduces levels of one or more of cytokines of IL-1α, IL-1SYMBOL, IL-2, IL-4, IL-6, IL-7, IL-8, IL-10, IL-12, IFN-SYMBOL, TNF-α, IP-10, MCP-1, MIP-1, RANTES, and GM-CSF in the subject.

Embodiment 319. The method of embodiment 300, wherein the first composition does not reduce a T cell-mediated killing of a cancer cell by more than about 5%.

Embodiment 320. The method of embodiment 300, wherein the cancer is B-cell lymphoma, aggressive, relapsed, or refractory diffuse large B cell lymphoma, primary mediastinal B-cell lymphoma, high grade B-cell lymphoma, transformed follicular lymphoma, relapsed or refractory mantle cell lymphoma, acute lymphoblastic leukemia, Hodgkin's lymphoma, non-Hodgkin's lymphoma, acute myeloid leukemia, or multiple myeloma.

Embodiment 321. The method of embodiment 300, wherein the cancer is brain cancer, breast cancer, glioblastoma, lung cancer, non-small-cell lung cancer, multiple myeloma, ovarian cancer, neuroblastoma, colorectal, biliary, pancreatic, mesothelioma, hepatoblastoma, embryonal sarcoma, prostate, sarcoma, or liver metastases.

Embodiment 322. The method of embodiment 300, wherein the cancer is a B-cell lymphoma.

Embodiment 323. The method of embodiment 300, wherein the subject is a mammal.

Embodiment 324. The method of embodiment 300, wherein the subject is a non-human primate, cat, dog, pig, cow, goat, horse, sheep, or rabbit.

Embodiment 325. The method of embodiment 300, wherein the subject is a human.

Embodiment 326. The method of embodiment 300, wherein the at least one bispecific antibody and the first composition are administered to the subject concurrently.

Embodiment 327. The method of embodiment 300, wherein the first composition is administered to the subject after the at least one bispecific antibody is administered to the subject.

Embodiment 328. The method of embodiment 300, wherein the first composition is administered to the subject starting about 1 day, about 2 days, about 3 days, about 4 days, about 5 days, about 6 days, or about 7 days after the at least one bispecific antibody is administered to the subject.

Embodiment 329. The method of embodiment 300, wherein the first composition is administered to the subject starting about 3 days to about 7 days after the at least one bispecific antibody is administered to the subject.

Embodiment 330. The method of embodiment 300, wherein the first composition is administered to the subject: after the at least one bispecific antibody is administered to the subject; and once onset of CRS is detected.

Embodiment 331. The method of embodiment 300, wherein the first composition is administered to the subject: after the at least one bispecific antibody is administered to the subject; and once onset of CRS is detected by an increased level of one or more of cytokine IL-1a, IL-b, IL-2, IL-4, IL-6, IL-7, IL-8, IL-10, IL-12, IFN-g, TNF-a, IP-10, MCP-1, MIP-1, RANTES, and GM-CSF.

Embodiment 332. The method of embodiment 300, wherein the first composition is administered to the subject: after the at least one bispecific antibody is administered to the subject; and once onset of CRS is detected by an increased level of one or more of cytokine IL-6, IL-10, IFN-g, and TNF-a.

Embodiment 333. The method of embodiment 300, wherein the first composition is administered for a period of about 1 day to about 30 days.

Embodiment 334. The method of embodiment 300, wherein the first composition is administered for a period of about 7 days to about 14 days.

Embodiment 335. The method of embodiment 300, further comprising administering the first composition to the subject before administration of the at least one bispecific antibody.

Embodiment 336. The method of embodiment 300, wherein the subject experiences reduced CRS relative to a similar subject receiving administered at least one bispecific antibody but not receiving the administered first composition.

Embodiment 337. The method of embodiment 300, wherein the subject does not experience CRS.

Embodiment 338. The method of embodiment 300, wherein the administering comprises oral delivery or intravenous injection (IV) delivery.

Embodiment 339. The method of embodiment 300, wherein the at least one bispecific antibody has a mechanism of action of recruitment and activation of immune cells, blocking of immune checkpoints, blocking of inflammatory factors, blocking of dual signal pathways, or combinations thereof.

Embodiment 340. The method of embodiment 300, wherein the at least one bispecific antibody is Blincyto (Blinatumomab), Hemlibra (emicizumab-kxwh), Rybrevant (amivantamab-vmjw), Kimmtrak (tebentafusp-tebn), or combinations thereof.

Embodiment 341. The method of embodiment 300, wherein the at least one bispecific antibody is a CD19-CD3 bispecific antibody, a Factor IX-Factor X bispecific antibody, an EGFR-MET bispecific antibody, a gp100-CD3 bispecific antibody, a BCMA-CD3 bispecific antibody, a CD20-CD3 bispecific antibody, a GPRC5D-CD3 bispecific antibody, or combinations thereof.

Embodiment 342. A method of treating cytokine release syndrome (CRS), immune effector cell-associated neurotoxicity syndrome (ICANS), or both associated with administration of at least one bispecific antibody in a subject, the method comprising administering at least one bispecific antibody to the subject, and administering a first composition to the subject; wherein the first composition comprises at least an effective amount of a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0%.

Embodiment 343. The method of embodiment 342, wherein the first composition does not contain more than about 1.0% of an isomer other than the compound of Formula I.

Embodiment 344. The method of embodiment 342, wherein the first composition does not contain more than about 0.15% of an isomer other than the compound of Formula I.

Embodiment 345. The method of embodiment 342, wherein the first composition does not contain more than about 0.1% of an isomer other than the compound of Formula I.

Embodiment 346. The method of embodiment 342, wherein the first composition does not contain more than about 0.05% of an isomer other than the compound of Formula I.

Embodiment 347. The method of embodiment 342, wherein the compound of Formula I is at least about 95% pure.

Embodiment 348. The method of embodiment 342, wherein the compound of Formula I is at least about 99% pure.

Embodiment 349. The method of embodiment 342, wherein the first compound of Formula I is at least about 99.8% pure.

Embodiment 350. The method of embodiment 342, wherein any individual impurity is present in an amount of less than about 0.15%.

Embodiment 351. The method of embodiment 342, wherein any individual impurity is present in an amount of less than about 0.1%.

Embodiment 352. The method of embodiment 342, wherein any individual impurity is present in an amount of less than about 0.05%.

Embodiment 353. The method of embodiment 342, wherein the first composition contains not more than about 0.5 wt % degradation product.

Embodiment 354. The method of embodiment 342, wherein the first composition contains not more than about 0.25 wt % degradation product.

Embodiment 355. The method of embodiment 342, wherein the first composition contains not more than about 0.1 wt % degradation product.

Embodiment 356. The method of embodiment 342, wherein the compound of Formula I is:

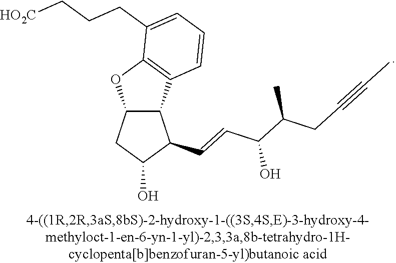 [SMILES: CC#CC[C@H](C)[C@H](O)/C=C/[C@@H]1[C@H]2c3cccc(CCCC(=O)O)c3O[C@H]2C[C@H]1O]

Embodiment 357. The method of embodiment 342, wherein the pharmaceutically acceptable salt of a compound of Formula I is:

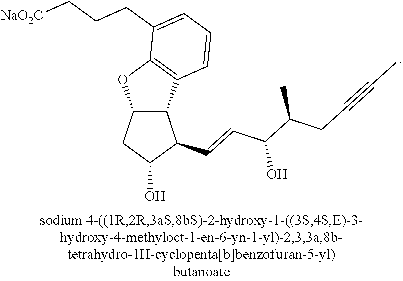

Embodiment 358. The method of embodiment 342, further comprising administering to the subject a second composition comprising at least one corticosteroid, tocilizumab, IL-6 receptor blocker, or combinations thereof.

Embodiment 359. The method of embodiment 342, further comprising administering to the subject a second composition comprising at least one corticosteroid, tocilizumab, IL-6 receptor blocker, or combinations thereof; wherein the subject requires reduced treatment with the second composition relative to a subject who does not receive the first composition.

Embodiment 360. The method of embodiment 342, wherein the first composition reduces levels of one or more of cytokines of IL-1α, IL-1SYMBOL, IL-2, IL-4, IL-6, IL-7, IL-8, IL-10, IL-12, IFN-SYMBOL, TNF-α, IP-10, MCP-1, MIP-1, RANTES, and GM-CSF in the subject.

Embodiment 361. The method of embodiment 342, wherein the CRS is a grade 1 CRS, a grade 2 CRS, a grade 3 CRS or a grade 4 CRS.

Embodiment 362. The method of embodiment 342, wherein the ICANS is a grade 1 ICANS, a grade 2 ICANS, a grade 3 ICANS or a grade 4 ICANS.

Embodiment 363. The method of embodiment 342, wherein the subject experiences reduced severity measurements associated with CRS, ICANS or both upon receiving the first composition relative to subject who does not receive the first composition.

Embodiment 364. The method of embodiment 342, wherein the subject is a mammal.

Embodiment 365. The method of embodiment 342, wherein the subject is a non-human primate, cat, dog, pig, cow, goat, horse, sheep, or rabbit.

Embodiment 366. The method of embodiment 342, wherein the subject is a human.

Embodiment 367. The method of embodiment 342, wherein the at least one bispecific antibody and the first composition are administered to the subject concurrently.

Embodiment 368. The method of embodiment 342, wherein the first composition is administered to the subject after the at least one bispecific antibody is administered to the subject.

Embodiment 369. The method of embodiment 342, wherein the first composition is administered to the subject starting about 1 day, about 2 days, about 3 days, about 4 days, about 5 days, about 6 days, or about 7 days after the at least one bispecific antibody is administered to the subject.

Embodiment 370. The method of embodiment 342, wherein the first composition is administered to the subject starting about 3 days to about 7 days after the at least one bispecific antibody is administered to the subject.

Embodiment 371. The method of embodiment 342, wherein the first composition is administered to the subject: after the at least one bispecific antibody is administered to the subject; and once onset of CRS is detected.

Embodiment 372. The method of embodiment 342, wherein the first composition is administered to the subject: after the at least one bispecific antibody is administered to the subject; and once onset of CRS is detected by an increased level of one or more of cytokine IL-1α, IL-1SYMBOL, IL-2, IL-4, IL-6, IL-7, IL-8, IL-10, IL-12, IFN-SYMBOL, TNF-α, IP-10, MCP-1, MIP-1, RANTES, and GM-CSF.

Embodiment 373. The method of embodiment 342, wherein the first composition is administered to the subject: after the at least one bispecific antibody is administered to the subject; and once onset of CRS is detected by an increased level of one or more of cytokine IL-6, IL-10, IFN-SYMBOL, and TNF-α.

Embodiment 374. The method of embodiment 342, wherein the first composition is administered for a period of about 1 day to about 30 days.

Embodiment 375. The method of embodiment 342, wherein the first composition is administered for a period of about 7 days to about 14 days.

Embodiment 376. The method of embodiment 342, further comprising administering the first composition to the subject before administration of the at least one bispecific antibody.

Embodiment 377. The method of embodiment 342, wherein the subject experiences reduced CRS relative to a similar subject receiving administered at least one bispecific antibody but not receiving the administered first composition.

Embodiment 378. The method of embodiment 342, wherein the subject does not experience CRS.

Embodiment 379. The method of embodiment 342, wherein the administering comprises oral delivery or intravenous injection (IV) delivery.

Embodiment 380. The method of embodiment 342, wherein the at least one bispecific antibody has a mechanism of action of recruitment and activation of immune cells, blocking of immune checkpoints, blocking of inflammatory factors, blocking of dual signal pathways, or combinations thereof.

Embodiment 381. The method of embodiment 342, wherein the at least one bispecific antibody is Blincyto (Blinatumomab), Hemlibra (emicizumab-kxwh), Rybrevant (amivantamab-vmjw), Kimmtrak (tebentafusp-tebn), or combinations thereof.

Embodiment 382. The method of embodiment 342, wherein the at least one bispecific antibody is a CD19-CD3 bispecific antibody, a Factor IX-Factor X bispecific antibody, an EGFR-MET bispecific antibody, a gp100-CD3 bispecific antibody, a BCMA-CD3 bispecific antibody, a CD20-CD3 bispecific antibody, a GPRC5D-CD3 bispecific antibody or combinations thereof.

Embodiment 383. A method of treating cytokine release syndrome (CRS), immune effector cell-associated neurotoxicity syndrome (ICANS), or both associated with CAR T-cell administration in a subject, the method comprising administering to the subject a population of CAR T-cells, and a first composition; wherein: the first composition comprises at least an effective amount of a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0%; and the first composition does not reduce a cell killing mediated by the population of CAR T-cells by more than about 5%.

Embodiment 384. The method of embodiment 383, wherein the first composition does not contain more than about 1.0% of an isomer other than the compound of Formula I.

Embodiment 385. The method of embodiment 383, wherein the first composition does not contain more than about 0.15% of an isomer other than the compound of Formula I.

Embodiment 386. The method of embodiment 383, wherein the first composition does not contain more than about 0.1% of an isomer other than the compound of Formula I.

Embodiment 387. The method of embodiment 383, wherein the first composition does not contain more than about 0.05% of an isomer other than the compound of Formula I.

Embodiment 388. The method of embodiment 383, wherein the compound of Formula I is at least about 95% pure.

Embodiment 389. The method of embodiment 383, wherein the compound of Formula I is at least about 99% pure.

Embodiment 390. The method of embodiment 383, wherein the first compound of Formula I is at least about 99.8% pure.

Embodiment 391. The method of embodiment 383, wherein any individual impurity is present in an amount of less than about 0.15%.

Embodiment 392. The method of embodiment 383, wherein any individual impurity is present in an amount of less than about 0.1%.

Embodiment 393. The method of embodiment 383, wherein any individual impurity is present in an amount of less than about 0.05%.

Embodiment 394. The method of embodiment 383, wherein the first composition contains not more than about 0.5 wt % degradation product.

Embodiment 395. The method of embodiment 383, wherein the first composition contains not more than about 0.25 wt % degradation product.

Embodiment 396. The method of embodiment 383, wherein the first composition contains not more than about 0.1 wt % degradation product.

Embodiment 397. The method of embodiment 383, wherein the compound of Formula I is:

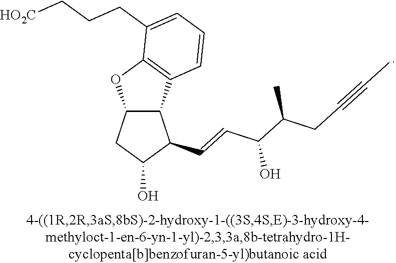 [SMILES: CC#CC[C@H](C)[C@H](O)/C=C/[C@@H]1[C@H]2c3cccc(CCCC(=O)O)c3O[C@H]2C[C@H]1O]

Embodiment 398. The method of embodiment 383, wherein the pharmaceutically acceptable salt of a compound of Formula I is:

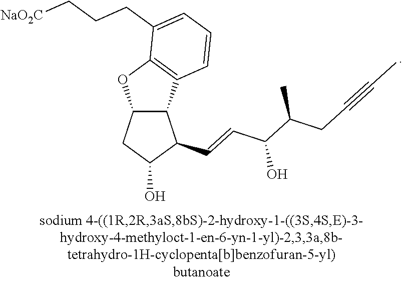

Embodiment 399. The method of embodiment 383, wherein the CRS is a grade 1 CRS, a grade 2 CRS, a grade 3 CRS, or a grade 4 CRS.

Embodiment 400. The method of embodiment 383, wherein the ICANS is a grade 1 ICANS, a grade 2 ICANS, a grade 3 ICANS, or a grade 4 ICANS.

Embodiment 401. The method of embodiment 383, wherein the first composition reduces levels of one or more of cytokines of IL-1α, IL-1SYMBOL, IL-2, IL-4, IL-6, IL-7, IL-8, IL-10, IL-12, IFN-SYMBOL, TNF-α, IP-10, MCP-1, MIP-1, RANTES, and GM-CSF in the subject.

Embodiment 402. The method of embodiment 383, further comprising administering a second composition comprising at least one corticosteroid, tocilizumab, IL-6 receptor blocker, or combinations thereof.

Embodiment 403. The method of embodiment 383, wherein the subject requires reduced treatment with the second composition relative to a subject who does not receive the first composition.

Embodiment 404. The method of embodiment 383, wherein the subject experiences reduced Parkinsonism effects upon receiving the first composition relative to a subject who does not receive the first composition.

Embodiment 405. The method of embodiment 383, wherein the subject experiences reduced severity measurements associated with CRS, ICANS or both upon receiving the first composition relative to subject who does not receive the first composition.

Embodiment 406. The method of embodiment 383, wherein the first composition is administered once onset of CRS is detected by an increased level of one or more cytokines of IL-6, IL-10, IFN-SYMBOL, TNF-α, MIF, IL-5, IL-17A, IL-23, CXCL9/MIG, GCSF, VEGF-A, and TGF-SYMBOL, or one or more of inflammatory biomarker C-reactive protein (CRP) and ferritin.

Embodiment 407. The method of embodiment 383, wherein: the CAR T-cell administration is performed to treat cancer; and the cancer is B-cell lymphoma, aggressive, relapsed or refractory diffuse large B cell lymphoma, primary mediastinal B-cell lymphoma, high grade B-cell lymphoma, transformed follicular lymphoma, relapsed or refractory mantle cell lymphoma, acute lymphoblastic leukemia, Hodgkin lymphoma, non-Hodgkin lymphoma, acute myeloid leukemia, or multiple myeloma.

Embodiment 408. The method of embodiment 383, wherein: the CAR T-cell administration is performed to treat cancer; and the cancer is brain cancer, breast cancer, glioblastoma, lung cancer, non-small-cell lung cancer, multiple myeloma, ovarian cancer, neuroblastoma, colorectal, biliary, pancreatic, mesothelioma, hepatoblastoma, embryonal sarcoma, prostate, sarcoma, or liver metastases.

Embodiment 409. The method of embodiment 383, wherein: the CAR T-cell administration is performed to treat cancer; and the cancer is B-cell lymphoma.

Embodiment 410. The method of embodiment 383, wherein the subject is a mammal.

Embodiment 411. The method of embodiment 383, wherein the subject is a non-human primate, cat, dog, pig, cow, goat, horse, sheep, or rabbit.

Embodiment 412. The method of embodiment 383, wherein the subject is a human.

Embodiment 413. The method of embodiment 383, wherein the first composition is administered to the subject before the population of CAR T-cells are administered to the subject.

Embodiment 414. The method of embodiment 402, wherein the second composition is administered to the subject after the population of CAR T-cells are administered to the subject.

Embodiment 415. The method of embodiment 402, wherein the first composition and the second composition are administered to the subject after the population of CAR T-cells are administered to the subject.

Embodiment 416. The method of embodiment 415, wherein the first composition and the second composition are administered concurrently.

Embodiment 417. The method of embodiment 402, wherein the first composition and the second composition are administered to the subject before the population of CAR T-cells are administered to the subject.

Embodiment 418. The method of embodiment 383, wherein the first composition is administered to the subject starting about 1 day, about 2 days, about 3 days, about 4 days, about 5 days, about 6 days, or about 7 days after the CAR T-cells are administered to the subject.

Embodiment 419. The method of embodiment 383, wherein the first composition is administered to the subject starting about 3 days to about 7 days after the population of CAR T-cells are administered to the subject.

Embodiment 420. The method of embodiment 383, wherein the first composition is administered for a period of about 1 day to about 30 days.

Embodiment 421. The method of embodiment 383, wherein the first composition is administered for a period of about 7 days to about 14 days.

Embodiment 422. The method of embodiment 383, wherein the first composition is administered starting one day before administration of the CAR T-cells and continued for a period of at least about 14 days.

Embodiment 423. The method of embodiment 383, wherein the CAR T-cells are autologous CAR T-cells.

Embodiment 424. The method of embodiment 383, wherein the CAR T-cells are allogeneic CAR T-cells.

Embodiment 425. The method of embodiment 383, wherein the first composition further comprises at least one excipient, at least one filler, at least one disintegrant, at least one binder, at least one wetting agent, at least one lubricant, at least one glidant, at least one preservative agent, at least one flavoring agent, at least one antioxidant, or combinations thereof.

Embodiment 426. The method of embodiment 383, wherein the first composition is formulated as a tablet, capsule, granule, powder, liquid, suspension, gel, syrup, slurry, suppository, patch, nasal spray, aerosol, injectable, implantable sustained-release formulation, or mucoadherent film.

Embodiment 427. The method of embodiment 383, wherein the administering comprises topical delivery, subcutaneous delivery, intravenous injection (IV) delivery, intramuscular injection (IM) delivery, intrathecal injection (IT) delivery, intraperitoneal injection (IP) delivery, transdermal delivery, subcutaneous delivery, oral delivery, transmucosal oral delivery, pulmonary delivery, inhalation delivery, intranasal delivery, buccal delivery, rectal delivery, vaginal delivery, or combinations thereof.

Embodiment 428. The method of embodiment 383, wherein the administering comprises oral delivery or intravenous injection (IV) delivery.

Embodiment 429. The method of embodiment 383, wherein the compound of Formula I, or pharmaceutically acceptable salt thereof is present in a unit dose of the first composition in an amount of about 1 microgram to about 100 micrograms.

Embodiment 430. The method of embodiment 383, wherein the administering comprises delivering the first composition to the subject at an amount of a compound of Formula I, or a pharmaceutically acceptable salt thereof, at least about 0.1 microgram.

Embodiment 431. The method of embodiment 383, wherein the administering comprises delivering the first composition to the subject at an amount of a compound of Formula I, or a pharmaceutically acceptable salt thereof at about 0.1 microgram to about 5000 micrograms.

Embodiment 432. The method of embodiment 383, wherein the administering comprises delivering the first composition to the subject at an amount of a compound of Formula I, or a pharmaceutically acceptable salt thereof at about 15 micrograms to about 90 micrograms.

Embodiment 433. The method of embodiment 383, wherein the administering comprises delivering the first composition to the subject at an amount of a compound of Formula I, or a pharmaceutically acceptable salt thereof at about 60 micrograms to about 360 micrograms.

Embodiment 434. The method of embodiment 383, wherein the population of CAR T-cells comprises a population of BCMA CAR-T cells, a population of CD19 CAR-T cells, a population of CD19-CD3 bispecific CAR-T cells, or combinations thereof.

Embodiment 435. A kit for the treatment of cancer in a subject, the kit comprising: a first container containing a first composition comprising a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0%; a second container containing at least one bispecific antibody; and instructions for the administration of the first composition to the subject.

Embodiment 436. The kit of embodiment 435, wherein the first composition does not contain more than about 1.0% of an isomer other than the compound of Formula I.

Embodiment 437. The kit of embodiment 435, wherein the first composition does not contain more than about 0.15% of an isomer other than the compound of Formula I.

Embodiment 438. The kit of embodiment 435, wherein the first composition does not contain more than about 0.1% of an isomer other than the compound of Formula I.

Embodiment 439. The kit of embodiment 435, wherein the first composition does not contain more than about 0.05% of an isomer other than the compound of Formula I.

Embodiment 440. The kit of embodiment 435, wherein the compound of Formula I is at least about 95% pure.

Embodiment 441. The kit of embodiment 435, wherein the compound of Formula I is at least about 99% pure.

Embodiment 442. The kit of embodiment 435, wherein the first compound of Formula I is at least about 99.8% pure.

Embodiment 443. The kit of embodiment 435, wherein any individual impurity is present in an amount of less than about 0.15%.

Embodiment 444. The kit of embodiment 435, wherein any individual impurity is present in an amount of less than about 0.1%.

Embodiment 445. The kit of embodiment 435, wherein any individual impurity is present in an amount of less than about 0.05%.

Embodiment 446. The kit of embodiment 435, wherein the first composition contains not more than about 0.5 wt % degradation product.

Embodiment 447. The kit of embodiment 435, wherein the first composition contains not more than about 0.25 wt % degradation product.

Embodiment 448. The kit of embodiment 435, wherein the first composition contains not more than about 0.1 wt % degradation product.

Embodiment 449. The kit of embodiment 435, wherein the compound of Formula I is:

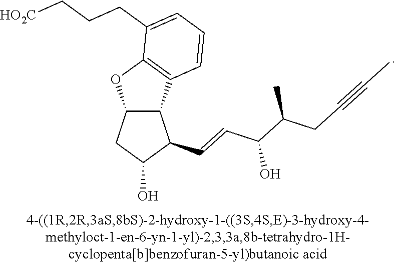 [SMILES: CC#CC[C@H](C)[C@H](O)/C=C/[C@@H]1[C@H]2c3cccc(CCCC(=O)O)c3O[C@H]2C[C@H]1O]

Embodiment 450. The kit of embodiment 435, wherein the pharmaceutically acceptable salt of a compound of Formula I is:

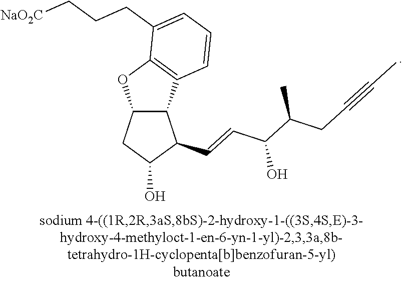

Embodiment 451. The kit of embodiment 435, further comprising a third container containing at least one corticosteroid, tocilizumab, IL-6 receptor blocker, or combinations thereof.

Embodiment 452. The kit of embodiment 435, further comprising a fourth container containing at least one solvent.

Embodiment 453. A kit for the treatment of cytokine release syndrome (CRS), immune effector cell-associated neurotoxicity syndrome (ICANS), or both in a subject, the kit comprising: a first container containing a first composition comprising a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0%; a second container containing at least one bispecific antibody; and instructions for the administration of the first composition to the subject.

Embodiment 454. The kit of embodiment 453, wherein the first composition does not contain more than about 1.0% of an isomer other than the compound of Formula I.

Embodiment 455. The kit of embodiment 453, wherein the first composition does not contain more than about 0.15% of an isomer other than the compound of Formula I.

Embodiment 456. The kit of embodiment 453, wherein the first composition does not contain more than about 0.1% of an isomer other than the compound of Formula I.

Embodiment 457. The kit of embodiment 453, wherein the first composition does not contain more than about 0.05% of an isomer other than the compound of Formula I.

Embodiment 458. The kit of embodiment 453, wherein the compound of Formula I is at least about 95% pure.

Embodiment 459. The kit of embodiment 453, wherein the compound of Formula I is at least about 99% pure.

Embodiment 460. The kit of embodiment 453, wherein the first compound of Formula I is at least about 99.8% pure.

Embodiment 461. The kit of embodiment 453, wherein any individual impurity is present in an amount of less than about 0.15%.

Embodiment 462. The kit of embodiment 453, wherein any individual impurity is present in an amount of less than about 0.1%.

Embodiment 463. The kit of embodiment 453, wherein any individual impurity is present in an amount of less than about 0.05%.

Embodiment 464. The kit of embodiment 453, wherein the first composition contains not more than about 0.5 wt % degradation product.

Embodiment 465. The kit of embodiment 453, wherein the first composition contains not more than about 0.25 wt % degradation product.

Embodiment 466. The kit of embodiment 453, wherein the first composition contains not more than about 0.1 wt % degradation product.

Embodiment 467. The kit of embodiment 453, wherein the compound of Formula I is:

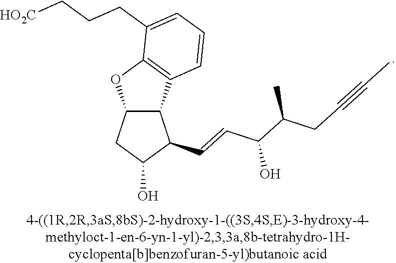 [SMILES: CC#CC[C@H](C)[C@H](O)/C=C/[C@@H]1[C@H]2c3cccc(CCCC(=O)O)c3O[C@H]2C[C@H]1O]

Embodiment 468. The kit of embodiment 453, wherein the pharmaceutically acceptable salt of a compound of Formula I is:

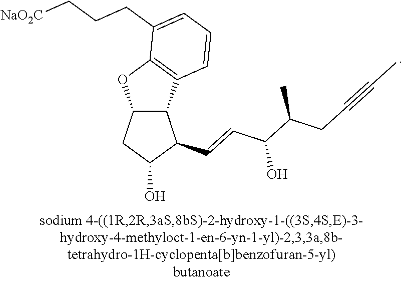

Embodiment 469. The kit of embodiment 453, further comprising a third container containing at least one corticosteroid, tocilizumab, IL-6 receptor blocker, or combinations thereof.

Embodiment 470. The kit of embodiment 453, further comprising a fourth container containing at least one solvent.

Embodiment 471. A kit for treating a subject with CAR T-cells, the kit comprising: a first container containing a first composition comprising a compound of Formula I, or a pharmaceutically acceptable salt thereof, wherein the compound of Formula I, or a pharmaceutically acceptable salt thereof, is at least about 90% pure and wherein any individual impurity is present in an amount of less than about 1.0%; a second container containing a second composition comprising at least one corticosteroid, tocilizumab, IL-6 receptor blocker, or combinations thereof; and instructions for the administration of the first composition, the second composition, and CAR T-cells to a subject.

Embodiment 472. The kit of embodiment 471, wherein the first composition does not contain more than about 1.0% of an isomer other than the compound of Formula I.

Embodiment 473. The kit of embodiment 471, wherein the first composition does not contain more than about 0.15% of an isomer other than the compound of Formula I.

Embodiment 474. The kit of embodiment 471, wherein the first composition does not contain more than about 0.1% of an isomer other than the compound of Formula I.

Embodiment 475. The kit of embodiment 471, wherein the first composition does not contain more than about 0.05% of an isomer other than the compound of Formula I.

Embodiment 476. The kit of embodiment 471, wherein the compound of Formula I is at least about 95% pure.

Embodiment 477 The kit of embodiment 471, wherein the compound of Formula I is at least about 99% pure.

Embodiment 478. The kit of embodiment 471, wherein the first compound of Formula I is at least about 99.8% pure.

Embodiment 479. The kit of embodiment 471, wherein any individual impurity is present in an amount of less than about 0.15%.

Embodiment 480. The kit of embodiment 471, wherein any individual impurity is present in an amount of less than about 0.1%.

Embodiment 481. The kit of embodiment 471, wherein any individual impurity is present in an amount of less than about 0.05%.

Embodiment 482. The kit of embodiment 471, wherein the first composition contains not more than about 0.5 wt % degradation product.

Embodiment 483. The kit of embodiment 471, wherein the first composition contains not more than about 0.25 wt % degradation product.

Embodiment 484. The kit of embodiment 471, wherein the first composition contains not more than about 0.1 wt % degradation product.

Embodiment 485. The kit of embodiment 471, wherein the compound of Formula I is:

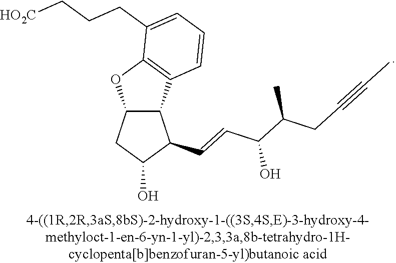 [SMILES: CC#CC[C@H](C)[C@H](O)/C=C/[C@@H]1[C@H]2c3cccc(CCCC(=O)O)c3O[C@H]2C[C@H]1O]

Embodiment 486. The kit of embodiment 471, wherein the pharmaceutically acceptable salt of a compound of Formula I is:

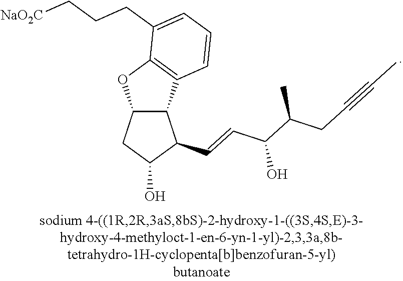

Embodiment 487. The kit of embodiment 471, further comprising a third container containing CAR T-cells.

Embodiment 488. The kit of embodiment 471, further comprising a fourth container containing water or an aqueous solution.

Embodiment 489: A compound, composition, or method as substantially described or substantially illustrated herein.

Embodiment 490: The method of embodiment 1, wherein, Formula I is prepared without the use of chiral chromatography.

Embodiment 491: The method of embodiment 14, wherein, sodium 4-((1R,2R,3aS,8bS)-2-hydroxy-1-((3S,4S,E)-3-hydroxy-4-methyloct-1-en-6-yn-1-yl)-2,3,3a,8b-tetrahydro-1H-cyclopenta[b]benzofuran-5-yl)butanoate is prepared without the use of chiral chromatography.

Embodiment 492: The method of embodiment 241, wherein, Formula I is prepared without the use of chiral chromatography.

Embodiment 493: The method of embodiment 254, wherein, sodium 4-((1R,2R,3aS,8bS)-2-hydroxy-1-((3S,4S,E)-3-hydroxy-4-methyloct-1-en-6-yn-1-yl)-2,3,3a,8b-tetrahydro-1H-cyclopenta[b]benzofuran-5-yl)butanoate is prepared without the use of chiral chromatography.

### Experimental Section

The compound of the present invention can, but are not limited to being prepared using the methods illustrated in the experimental procedures detailed below. The starting materials used to prepare the compounds of the present invention are commercially available or can be prepared using routine methods known in the art. Solvents and reagents, whose synthetic preparations are not described below, can be purchased at Sigma-Aldrich or Fisher Scientific.

Representative procedures for the preparation of compounds of this disclosure are outlined below.

The preparation of the present invention is described in more detail referring to the following Scheme 1. The process depicted in the following Scheme 1 represents merely a typical example, and various changes may be made to reagents and reaction conditions without limitation.

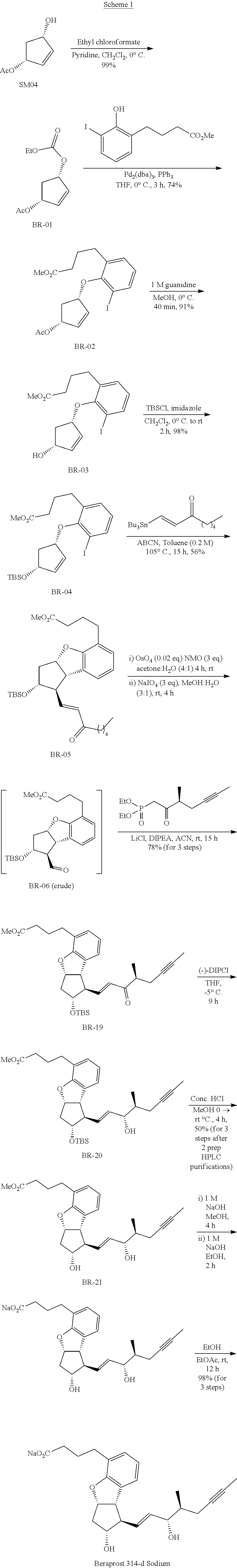

Stork and co-workers have previously reported a radical cyclization-trapping method for the construction of the PGF2a framework shown in Scheme 2 (J. Am. Chem. Soc. 1986, 108, 6384-6385; J. Am. Chem. Soc. 1986, 108, 304-305).

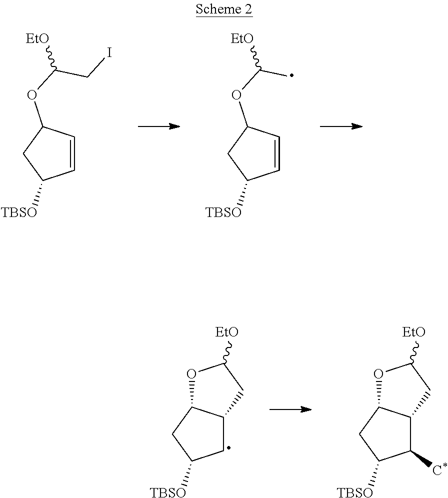

Keck and Burnett later improved upon the Stork procedure by employing a 3-stannyl enone as a radical trapping reagent shown in Scheme 3 (J. Org. Chem. 1987, 52, 2960-2962).

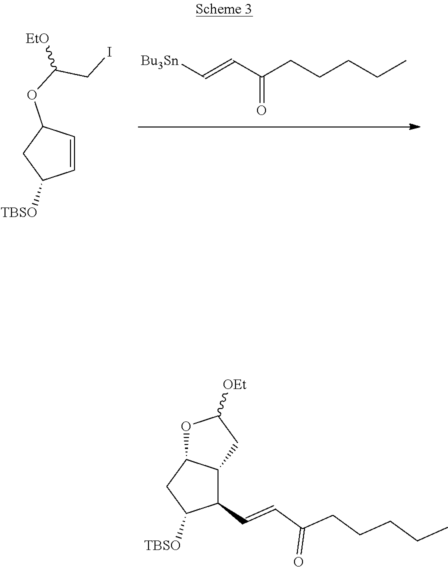

Employment of this strategy to benzoprostacyclins using the radical precursor (no protecting group at C11) shown in Scheme 4 had been regarded as a successful route by Larock and co-workers (J. Org. Chem. 1991, 56, 6253-6254; Bull. Korean Chem. Soc. 2001, 22, 857).

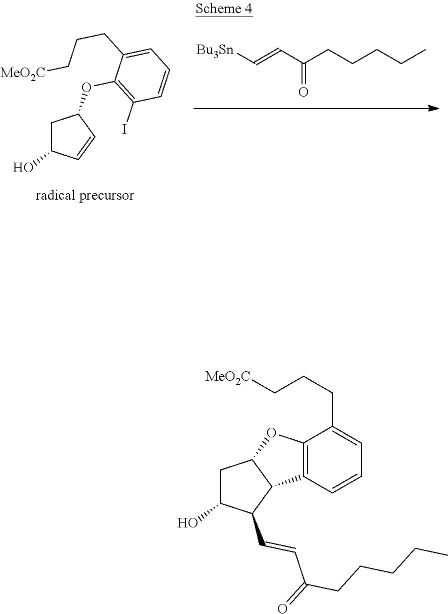

Surprisingly, during our process improvement to KR Pat. No. 10-1777633, we obtained unrequired 12-epimer as a major product in radical cyclization shown in Scheme 5.

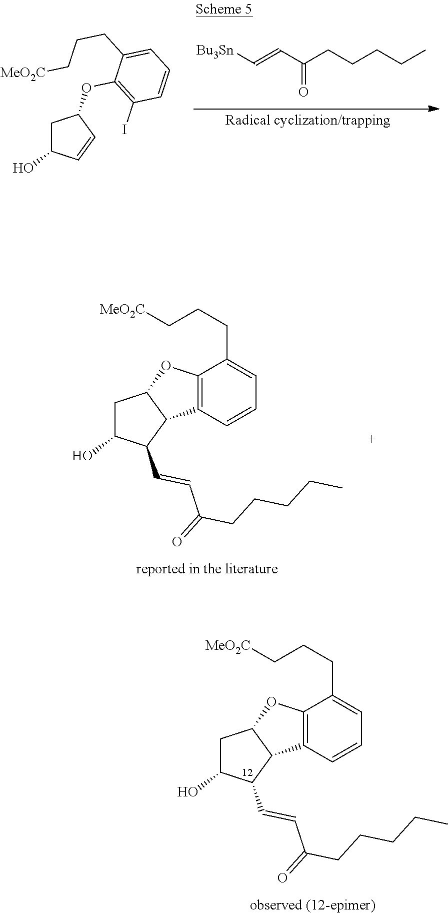

A thorough analysis of the data (nOe analysis) from the literature reported by Larock and co-workers (J. Org. Chem. 1991, 56, 6253-6254; Bull. Korean Chem. Soc. 2001, 22, 857) revealed that radical cyclization occurs under chelation-controlled fashion (Scheme 6), which produces only 12-epimer as a single diastereomer. Therefore, the reactions in both Scheme 4 and Scheme 5 were revised.

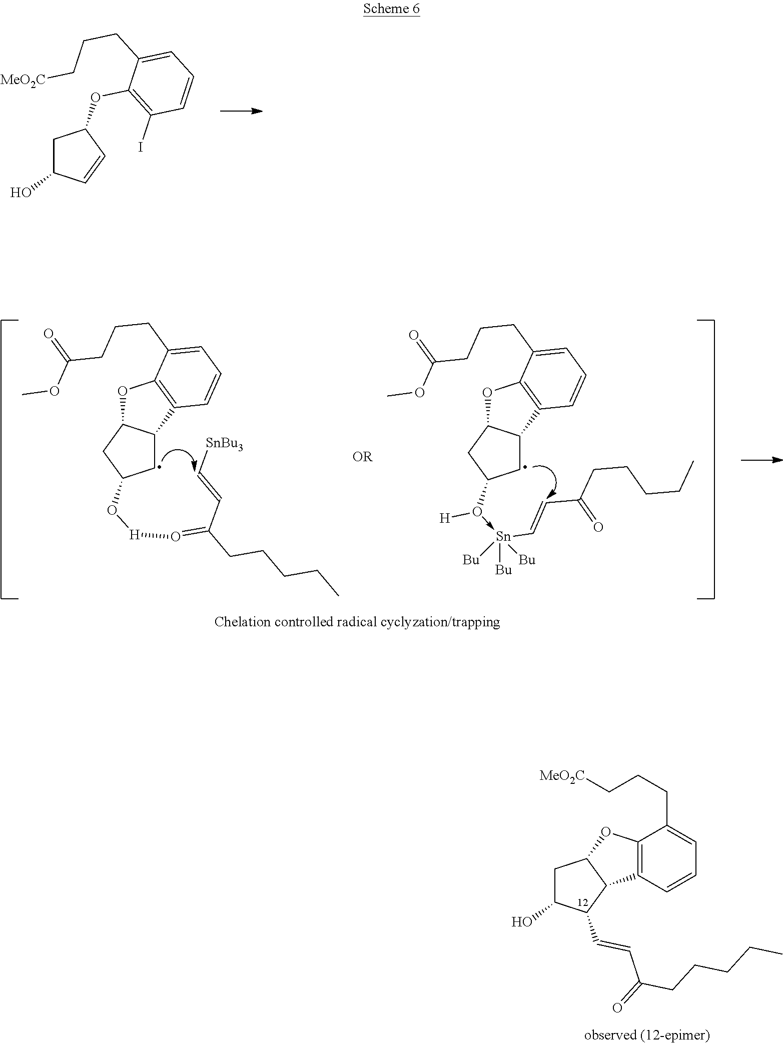

After treating the radical precursor, 10-20% of the desired product is formed (Scheme 7). Further optimizing the radical cyclization by changing the parameters like the radical initiator, reaction concentration, equivalents of β-stannylenone, and temperature resulted in a satisfactory yield (Table 1).

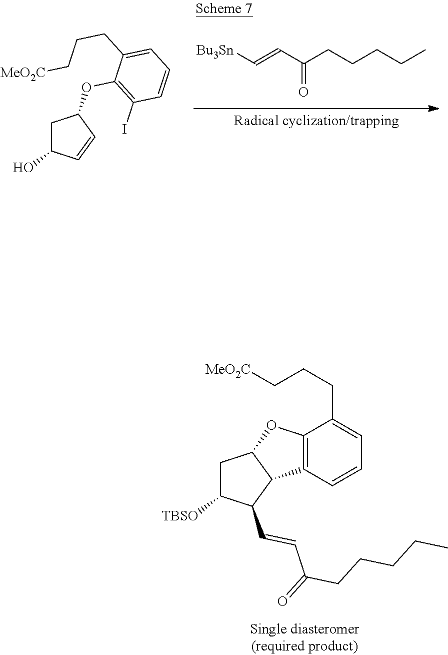

### Optimization of the Radical Cyclization Reaction

TABLE 1

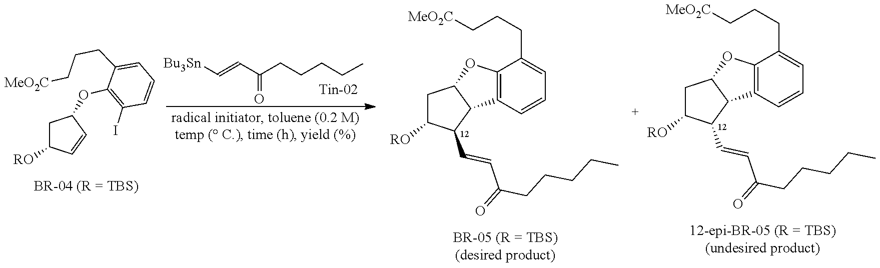

| En- | | Tin-02 | Radical initiator | | | Desired | 12-Epimer |
|---|---|---|---|---|---|---|---|
| try | R | (equiv.) | (equiv.) | Temp [° C.] | Time [h] | product [%]¹ | [%]¹ |
| 1² | H | 2.0 | AIBN (0.08) | 80 | no info | ND | 95 |
| 2 | H | 3.0 | AIBN (0.1) | 90 | 3 | ND | 84 |
| 3 | TBS | 4.0 | AIBN (0.1) | 90 | 24 | Trace³ | ND |
| 4 | TBS | 4.0 | AIBN (0.2) | 110 | 24 | 31³ | ND |
| 5 | TBS | 4.0 | ABCN (0.1) | 110 | 15 | 45³ | ND |
| 6 | TBS | 4.0 | ABCN (0.1) | 110 | 15 | 54 | ND |
| 7 | TBS | 4.0 | ABCN (0.1) | 95-100 | 15 | 55 | ND |
| 8 | TBS | 4.0 | ABCN (0.1) | 84-87 | 15 | 62⁴ | ND |
| 9 | TBS | 4.0 | ABCN (0.1) | 84-87 | 20 | 61 | ND |
| 10 | TBS | 4.0 | ABCN (0.1) | 74-78 | 15 | NR | ND |
| 11 | TBS | 4.0 | ABCN (0.1) | 84-87 | 15 | 66⁵ | ND |
| 12 | TBS | 4.0 | ABCN (0.1) | 102-104 | 15 | 57 | ND |

Reaction conditions: Reactions were performed on a 0.2 mmol scale in a sealed tube (except entry 1). [1] Isolated yield after column chromatography; [2] Refer the synthesis of compound 13 in KR10-1777633 (see [0087] and [0147]-[0149] of KR10-1777633). ¹H-NMR spectra of [0149] of KR10-1777633 imply 12-epimer, however, relative stereochemistry was incorrectly assigned there; [3] Toluene (0.1 M); [4] Sparging toluene for 5 mins; [5] Sparging toluene for 15 mins. ND=Not Detected.

The key radical cyclization-trapping sequence proved to be more sensitive to reaction conditions (particularly temperature) than expected. Heating a toluene solution (0.1 M in substrate) of the iodo benzene (BR-04) containing 4 equiv. of β-stannylenone (tin-02) and 0.1 equiv. of azobisisobutyronitrile (AIBN) at 90° C. for extended periods gave no more than trace amounts of the desired product BR-05 (entry 3). Conducting the reaction at 110° C. with 0.2 equiv. of AIBN for 24 h afforded the desired material, but in only 31% isolated yield after purification by column chromatography (entry 4). However, simply performing the reaction at 110° C. (toluene reflux) with azobiscyclohexylnitrile (ABCN) as the radical initiator and 4.0 equiv. of enone (tin-02) gave BR05 in a 45% isolated yield after purification by column chromatography (entry 5).

Increasing the reaction concentration from 0.1 M to 0.2 M, provided the desired product BR-05 in a 54% yield (entry 6). Further control experiments (1) sparging toluene solvent with argon and (2) at an ideal temperature range of 84-87° C. yielded a radical precursor with a yield of 66% (entry 11).

Step 1: Synthesis of Compound of Formula BR-02—The transformation of SM04 to carbonate BR-01 and subsequent Pd-catalyzed decarboxylative o-alkylation with C206 afforded the cis-product BR-02 in a 73% overall yield (for 2 steps) with a single diastereomer. It is worth commenting that the iodide functionality in compound BR-01 remained intact under the reaction conditions employed.

Step 2: Synthesis of Compound of Formula BR-03—Acetyl deprotection with guanidine of BR-02 afforded BR-03 in 91% yield.

Step 3: Synthesis of Compound of Formula BR-04—TBS protection of BR-03 provided the radical precursor BR-04 for radical cyclization in high yield.

Step 4: Synthesis of Compound of Formula BR-05—The reaction of BR-04 with trans-1-(tri-n-butylstannyl)-oct-1-en-3-one (tin-02) and 1,1′-Azobis(cyclohexanecarbonitrile) (ABCN) produced the key tricyclic intermediate BR-05 in 56% yield at 105° C. in toluene (0.2 M). Mechanistically, this process is believed to involve the formation of an aryl radical of BR-04 that in turn self-adds to produce a free radical at nascent position 12. This active species reacts in situ across the C—Sn bond of tin-02 to yield BR-05.

Step 5: Synthesis of Compound of Formula BR-19—The oxidative cleavage of key tricyclic intermediate BR-05 gave the corresponding aldehyde, which was used directly after an aqueous workup. The subsequent Horner-Wadsworth-Emmons reaction proceeded smoothly to deliver enone BR-19 in 78% yield over three steps together with perfect E-selectivity without any epimerization at C-16 chiral center.

Step 6: Synthesis of Compound of Formula BR-21—A diastereoselective 1,2-reduction of BR-19 with (−)-B-chlorodiisopinocampheylborane (DIP-Cl) (dr>90:10 at C-15) followed by TBS deprotection resulted in an allyl alcohol BR-21 with all of the required stereogenic centers.

Step 7: Synthesis of Compound of Formula (1), Beraprost 314-d Sodium—Hydrolysis of methyl ester BR-21 and sodium salt formation was afforded Formula (1), beraprost 314-d sodium in 98% yield.

Highlight of this invention is a concise, highly stereoselective, and short synthesis of beraprost 314-d sodium, which is the most biologically active isomer of beraprost sodium, from commercially available starting materials. The present synthesis has several notable features: (1) Highly diastereoselective Pd-Catalyzed decarboxylative o-alkylation; (2) Radical cyclization/trapping to get key tricyclic intermediate; (3) Highly E-selective HWE reaction without epimerization at C-16 chiral center; (4) Diastereoselective reduction with (−)-DIP-Cl; (5) Highly enantiomerically pure beraprost 314-d obtained; (6) 7 pots synthesis with only 6 column chromatographic purifications with an overall yield of 14%. The present synthesis contrasts with non-stereoselective syntheses of beraprost which require the isolation of the biologically active isomer beraprost 314-d via chiral column chromatography. The enantiomeric separation can prove difficult as chiral columns may degrade over time thus reducing the efficiency of the enantiomeric separation. The loss of efficiency in the enantiomeric separation results in material containing isomers other than beraprost 314-d. The present stereoselective synthesis avoids this problem as it does not require any enantiomeric separations via chiral chromatography.

### Example 1: Beraprost-314d Sodium Tablet Formulation

Beraprost-314d sodium tablets were prepared according to the following method with the composition shown in Table 2 below. A mixed solution was prepared by dissolving 0.05 g of beraprost-314d sodium in 90 g of purified water, and putting 40 g of microcrystalline cellulose, 271.45 g of lactose hydrate, 75 g of pregelatinized starch, and 10 g of hydroxypropylcellulose into a 2 L speed mixer at an impeller speed of 370 rpm. Granules are obtained through wet granulation by using the mixed liquid in this mixture, and then dried. After setting the dried granules to a certain size, 3.5 g of magnesium stearate was added and blended. The obtained final mixture was tableted in a standard amount of 80 mg, and the obtained uncoated tablets were put into a coater and film-coated with an HPMC-based coating agent to obtain beraprost-314d sodium tablets.

TABLE 2

| Process | Classification | Materials | g/batch |
|---|---|---|---|
| Intra-Granular | Active agent | Beraprost 314-d | 0.05 |
| Solution | | sodium | |
| | Solution | Purified water | 90 |
| Intra-Granular | Excipient | Microcrystalline | 40 |
| | | cellulose | |
| | | Lactose hydrate | 271.45 |
| | Disintegrant | Pregelatinized starch | 75 |
| | Binder | Hydroxypropylcellulose | 10 |
| Extra-Granular | Lubricant | Magnesium stearate | 3.5 |
| Coating | Coating | 03F690002 | 15 |
| | agent | | |
| | Solution | Purified water | 135 |

A content uniformity test was conducted on the tablets prepared according to Example 2 in order to confirm whether the content uniformity of the preparation was secured according to the manufacturing method. The content uniformity test was measured by liquid chromatography (HPLC), and the analysis was performed according to the following conditions:

### Assay Method:

Mobile phase: A mixture of methanol, water and acetic acid (750:250:1)
Diluent: A mixture of water and methanol (7:3)

### Chromatographic System:

Detector: UV 285 nm
Column: A stainless steel column 4.6 mm in inside diameter and 150 mm in length, packed with octadecylsilanized silica gel for liquid chromatography.
Flow rate: 0.8 mL/min
Oven: 35±2° C.
Injection volume: 100 μL
Run time: 10 min

### System Suitability:

Sample: standard solution
Suitability requirements
Tailing factor: NMT 2.0
Relative standard deviation: NMT 2.0%

Results are shown in Table 3. The L1 value of the tablet prepared by this method was within 15, proving content uniformity.

TABLE 3

| | Assay (%) |
|---|---|
| 1 | 99.0 |
| 2 | 102.5 |
| 3 | 102.1 |
| 4 | 104.8 |
| 5 | 101.2 |
| 6 | 101.8 |
| 7 | 102.4 |
| 8 | 100.9 |
| 9 | 102.0 |
| 10 | 100.4 |
| Average | 101.8 |
| S.D | 1.76 |
| RSD(%) | 1.73 |
| L1 | 4.23 |

### Example 2: Beraprost-314d Sodium Tablet Formulation with Binder Solution

Beraprost-314d sodium tablets were prepared according to the following method with the composition shown in Table 4 below. 0.05 g of Beraprost-314d sodium and 2.5 g of Hydroxypropyl methylcellulose were dissolved in 72.5 g of purified water to prepare a mixed solution, and 278.7 g of Lactose hydrate, 110 g of Corn starch and 7.5 g of Hydroxypropyl methylcellulose were put into a 2 L speed mixer and mixed at an impeller speed of 370 rpm. Granules are obtained through wet granulation by using the mixed liquid in this mixture, and then dried. After setting the dried granules to a certain size, 1.25 g of Magnesium stearate was added and blended. The obtained final mixture was tableted in a standard amount of 80 mg, and the obtained uncoated tablets were put into a coater and film-coated with an HPMC-based coating agent to obtain Beraprost-314d sodium tablets.

TABLE 4

| Process | Classification | Materials | g/batch |
|---|---|---|---|
| Binder solution | Active agent | Beraprost 314-d | 0.05 |
| Intra-Granular | | sodium | |
| | Binder | Hydroxypropyl | 2.5 |
| | | methylcellulose | |
| | Solution | Purified water | 72.5 |
| Intra-Granular | Diluent | Lactose hydrate | 278.7 |
| | Disintegrant | Corn starch | 110 |
| | Binder | Hydroxypropyl | 7.5 |
| | | methylcellulose | |
| Extra-Granular | Lubricant | Magnesium stearate | 1.25 |
| Coating | Coating | 03F690002 | 15 |
| | agent | | |
| | Solution | Purified water | 135 |

In order to confirm whether the content uniformity of the preparation according to the manufacturing method was secured, a content uniformity test was performed on the tablets prepared according to the prescription of Example 2. The content uniformity test was measured by liquid chromatography (HPLC), and the analysis was performed according to the following conditions:

### Assay Method:

Mobile phase: A mixture of methanol, water and acetic acid (750:250:1)
Diluent: A mixture of water and methanol (7:3)
   Chromatographic system
Detector: UV 285 nm
Column: A stainless steel column 4.6 mm in inside diameter and 150 mm in length, packed with octadecylsilanized silica gel for liquid chromatography
Flow rate: 0.8 mL/min
Oven: 35±2° C.
Injection volume: 100 μL
Run time: 10 min.
   System suitability
Sample: standard solution
   Suitability requirements
Tailing factor: NMT 2.0
Relative standard deviation: NMT 2.0%

The test results for Example 2 are shown in Table 5. The L1 value of the tablet prepared by the prescription of Example 2 was within 15, proving content uniformity.

TABLE 5

| | Assay (%) |
|---|---|
| 1 | 100.6 |
| 2 | 101.8 |
| 3 | 101.8 |
| 4 | 102.5 |
| 5 | 100.1 |
| 6 | 103.8 |
| 7 | 100.2 |
| 8 | 100.4 |
| 9 | 100.8 |
| 10 | 99.3 |
| average | 101.1 |
| S.D | 1.34 |
| RSD(%) | 1.33 |
| L1 | 3.22 |

### Example 3: Synthesis of Compound of Formula BR-02—methyl 4-(2-(((1S,4R)-4-acetoxycyclopent-2-en-1-yl)oxy)-3-iodophenyl)butanoate

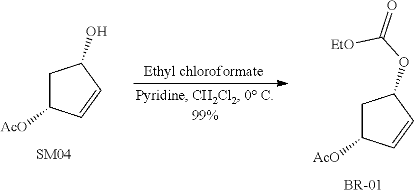 [SMILES: CC(=O)O[C@H]1C=C[C@@H](O)C1 ; CCOC(=O)O[C@@H]1C=C[C@H](OC(C)=O)C1]

150 g (1.055 mol, 1.0 eq) of (1R,4S)-4-hydroxycyclopent-2-en-1-yl acetate was dissolved in 1.5 L (10 v/w, 0.7 M) of DCM in a 3-neck 3 L RBF under an argon atmosphere and cooled to 0° C. (internal temp). After that, pyridine 426.7 mL (5.28 mol, 5.0 eq) was added dropwise over 20 mins, followed by 302.6 mL (3.16 mol, 3.0 eq) of ethyl chloroformate added dropwise using an addition funnel over 1 h (during addition, internal temperature was raised, so always maintain the internal temperature below 5° C.). Stirred for 0 to 5° C. for 2 h. After TLC check (EA:Hex=1:4), confirmed starting consumption and then quenched the reaction with 1.5 L (10 v/w) of 1N HCl at 0 to 5° C. over 1 h (during addition, internal temperature was raised, so always maintain the internal temperature below 5° C.) and after stirring for 5 min warmed to room temperature and stirred for another 30 mins. The layers were separated, and the aqueous layer was extracted with 5 V of DCM (750 mL). The combined organic layers were transferred to a RB flask, and 5 v of 1N HCl (750 mL) was added at room temperature. The mixture was stirred at room temperature for 30 minutes, and the layers were separated (this process was repeated once more). The separated organic layer was washed with saturated aq. of NaHCO₃ solution (1.2 L, 8 V). The separated organic layer was dried on Na₂SO₄, filtered and concentrated on rotavapor at 30° C. The crude (oil, quantitative yield) of BR-01 ((1R,4S)-4-((ethoxycarbonyl)oxy)cyclopent-2-en-1-yl acetate) thus obtained is used in the next reaction without additional purification after dried on high vacuum for 24 to 48 hours (NMR). (TLC: EA:Hex (1:4), R_{f}=0.55). ¹H NMR (300 MHz, CDCl₃): δ 6.16-6.08 (m, 2H), 5.57-5.43 (m, 2H), 4.20 (q, J=7.1 Hz, 2H), 2.89 (dt, J=15.0, 7.5 Hz, 1H), 2.04 (s, 3H), 1.83 (dt, J=15.0, 3.8 Hz, 1H), 1.31 (t, J=7.1 Hz, 3H); ¹³C NMR (75 MHz, CDCl₃): δ 170.77, 154.75, 135.25, 134.12, 79.95, 76.44, 64.12, 37.07, 21.14, 14.33.

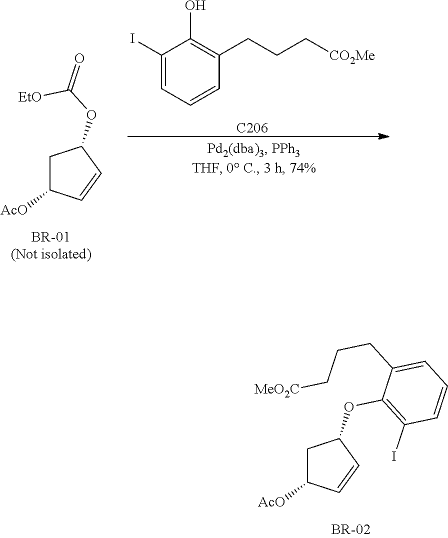 [SMILES: "C206" ; CCOC(=O)O[C@@H]1C=C[C@H](OC(C)=O)C1 ; COC(=O)CCCc1cccc(I)c1O ; COC(=O)CCCc1cccc(I)c1O[C@@H]1C=C[C@H](OC(C)=O)C1]

BR-01 (150 g, 0.70 mol) was transferred to a 5 L 4-neck RB flask, evacuated, and backfilled with argon. Degassed THF (1.05 L, 7 v/w) was added and cooled to 0° C. (internal temp). In another RB flask, C206 (336.2 g, 1.05 mol) was dissolved in degassed THF (1.5 L, 10 v/w) and added dropwise at 0° C. to the above reaction mixture. Once the internal temperature become 0° C., PPh₃ (146.6 g, 0.56 mol) followed by Pd₂(dba)₃ (64.1 g, 0.070 mol) were added in one portion. Maintain the internal temperature at 0° C. and stirred for 3 hours. Upon completion of the reaction, the cold reaction mixture was added over 1.5 L of cold saturated NaHCO₃ solution (10 V) with stirring over 5 to 10 mins, warmed to RT, diluted with 10 V of diethyl ether (1.5 L) and H₂O (1.5 L, 10 V), stirred at RT for 30 mins and the layers were separated. The aqueous layer was extracted with 10 V of diethyl ether (1.5 L) and the combined ether layers were washed with sat.NaHCO₃ solution (5 V×2), followed by brine (1.5 L, 10 V). Dried on Na₂SO₄, filtered, and concentrated under reduced pressure. Purified by column chromatography using ethyl acetate/hexane. (TLC: EA:Hex (1:4), R_{f}=0.5). ¹H NMR (300 MHz, CDCl₃): 7.67 (dd, J=7.8, 1.6 Hz, 1H), 7.17 (dd, J=7.6, 1.6 Hz, 1H), 6.79 (t, J=7.7 Hz, 1H), 6.22-6.15 (m, 1H), 6.09-6.03 (m, 1H), 5.57-5.47 (m, 1H), 5.13-5.04 (m, 1H), 3.66 (s, 3H), 2.93 (dt, J=14.7, 7.4 Hz, 1H), 2.85-2.59 (m, 2H), 2.31 (t, J=7.5 Hz, 2H), 2.19-2.07 (m, 4H), 1.98-1.85 (m, 2H).

### Example 4: Synthesis of Compound of Formula BR-03—methyl 4-(2-(((1S,4R)-4-hydroxycyclopent-2-en-1-yl)oxy)-3-iodophenyl)butanoate

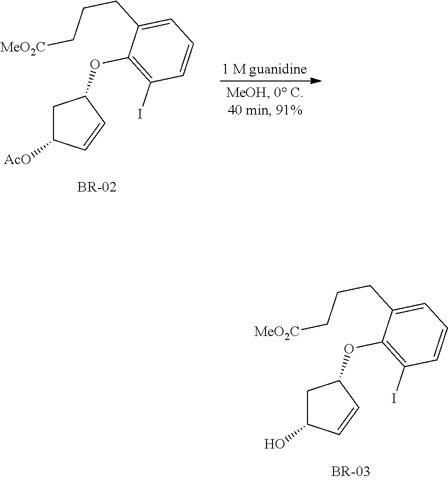 [SMILES: COC(=O)CCCc1cccc(I)c1O[C@@H]1C=C[C@H](OC(C)=O)C1 ; COC(=O)CCCc1cccc(I)c1O[C@@H]1C=C[C@H](O)C1]

A stock solution of 500 mL of 1.0 M guanidine in CH₃OH was prepared by adding 114 mL of NaOMe (25 wt %) solution dropwise (over 30 mins) to ice-cooled solution of guanidine carbonate (45 g, 0.499 mol) in CH₃OH (386 mL) under argon atmosphere. This solution was stirred at room temperature for 30 min, and the mixture was allowed to stand to settle out precipitated salts. In a separate RB flask was placed 240 g (0.540 mol) of BR-02 in 2.4 L of MeOH under argon. This was cooled to 0° C. (internal temp), and to it was added via syringe 270 mL of 1.0 M guanidine in CH₃OH (0.270 mol, 0.5 equiv.) prepared above, over 30 min. This mixture was stirred at 0° C. (internal temp) for 40 min. TLC showed complete consumption of acetate. To the reaction mixture was then added 15.46 mL (0.270 mol, 0.5 equiv.) of glacial acetic acid to neutralize the guanidine 0° C. over 10 min. After the mixture was stirred for 5 min, solvent was removed at reduced pressure to give a thick slurry. The residue was partitioned between 2.4 L of water (10 V) and 2.4 L of ethyl acetate (10 V). The aqueous layer was further extracted with 5 V of ethyl acetate. The combined organic layers were washed with 10 V of water (2.4 L) and 10 V of brine (2.4 L) and dried over sodium sulfate. Removal of solvent at reduced pressure gave a yellow oil, which was purified by flash chromatography on silica gel with ethyl acetate in hexane to give 180.1 g of BR-03 (91%) after removal of solvent. (TLC: EA:DCM (1:99), R_{f}=0.2). ¹H NMR (300 MHz, CDCl₃): δ 7.65 (dd, J=7.8, 1.6 Hz, 1H), 7.15 (dd, J=7.6, 1.6 Hz, 1H), 6.76 (t, J=7.7 Hz, 1H), 6.13-5.58 (m, 2H), 5.14-5.05 (m, 1H), 4.68 (brs, 1H), 3.66 (s, 3H), 2.91-2.77 (m, 2H), 2.73-2.53 (m, 2H), 2.40-2.21 (m, 2H), 2.05 (dt, J=14.4, 4.0 Hz, 1H), 1.99-1.75 (m, 2H); ¹³C NMR (75 MHz, CDCl₃): δ 174.25, 156.31, 138.18, 138.06, 136.61, 133.61, 130.62, 125.96, 92.53, 85.85, 74.94, 51.78, 41.33, 33.25, 30.83, 25.78.

### Example 5: Synthesis of Compound of Formula BR-04—methyl 4-(2-(((1S,4R)-4-((tert-butyldimethylsilyl)oxy)cyclopent-2-en-1-yl)oxy)-3-iodophenyl)butanoate

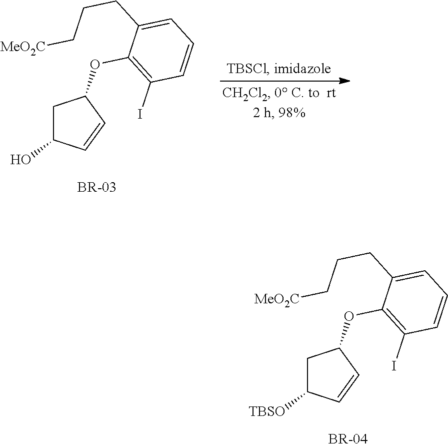 [SMILES: COC(=O)CCCc1cccc(I)c1O[C@@H]1C=C[C@H](O)C1 ; COC(=O)CCCc1cccc(I)c1O[C@@H]1C=C[C@H](O[Si](C)(C)C(C)(C)C)C1]

BR-03 (206 g, 0.512 mol) was transferred to a 3 L 4-neck RB flask, evacuated, backfilled with argon and 2 L of anhydrous DCM (10 V) was added and cooled to 0˜5° C. (internal temp). 52.3 g (0.768 mol, 1.5 eq) of imidazole was added in one portion. Then, 115.8 g of (0.768 mol, 1.5 eq) of TBSCl was added portion wise over 30 mins (during addition, internal temperature was raised, so always maintain the internal temperature below 5° C.). Warmed to RT and stirred at RT for 2 hours. After completion of the reaction, cooled to 0-5° C. (internal temp) and quenched with H₂O over 30 mins (during addition, internal temperature was raised, so always maintain the internal temperature below 5° C.). The organic layer was separated, and the aqueous layer was extracted with 1 L (5 v/w) of DCM. The combined organic layers were washed with brine (1 L, 10 V) and dried on Na₂SO₄, filtered, and concentrated under reduced pressure. The crude product was purified by column chromatography using ethyl acetate/hexane. (BR-04, 258 g, 98% yield; colorless liquid). (TLC: EA:Hex (1:4), R_{f}=0.8). ¹H NMR (300 MHz, CDCl₃): δ 7.66 (dd, J=7.8, 1.3 Hz, 1H), 7.16 (dd, J=7.5, 1.1 Hz, 1H), 6.77 (t, J=7.7 Hz, 1H), 5.96 (dd, J=17.3, 5.7 Hz, 2H), 5.02 (t, J=6.2 Hz, 1H), 4.68 (t, J=5.8 Hz, 1H), 3.65 (s, 3H), 2.90-2.59 (dt, J=16.5, 7.5 Hz, 2H), 2.74-2.58 (m, 1H), 2.31 (t, J=7.5 Hz, 2H), 2.06-1.83 (m, 3H), 0.92 (s, 9H), 0.11 (s, 6H). ¹³C NMR (75 MHz, CDCl₃): δ 173.99, 156.58, 138.14 (2C), 136.69, 132.67, 130.52, 125.88, 92.58, 86.05, 74.74, 51.66, 42.23, 33.61, 30.57, 26.04, 25.59, 18.33, −4.42, −4.48.

### Example 6: Synthesis of Compound of Formula BR-05—methyl 4-((1R,2R,3aS,8bS)-2-((tert-butyldimethylsilyl)oxy)-1-((E)-3-oxooct-1-en-1-yl)-2,3,3a,8b-tetrahydro-1H-cyclopenta[b]benzofuran-5-yl)butanoate

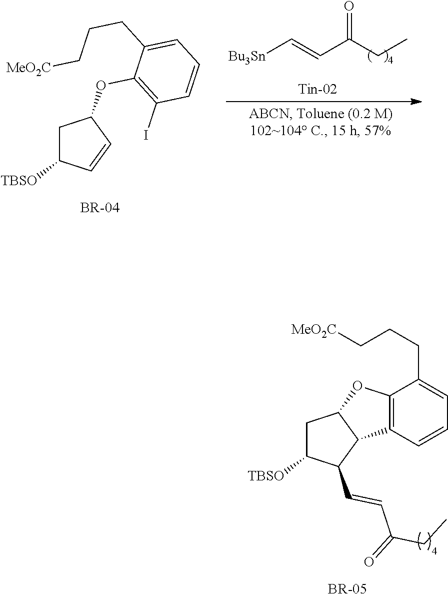

BR-04 (100 g, 0.194 mol) and 321 g of Tin-02 (0.774 mol, 4 equiv.) was transferred to a 2 L 3-neck RB flask, evacuated, backfilled with argon and 1 L of degassed (degassed for 1 hour prior to use) toluene (10 V, 0.2 M) was added. The resulting mixture was sparged with argon for 1 hour and ABCN (0.0194 mol, 0.1 equiv.) wad added. The reaction mixture was heated (reflux) to 102˜104° C. (internal temperature must be maintain above 102° C.) for 15 hours (don't use rubber septum during reflux). TLC indicating complete consumption of BR-04. Cooled to RT, toluene was evaporated on rotavapor at 45° C. and dried for 30 mins. The crude product was directly loaded (used small amount of toluene for loading) to column and purified using ethyl acetate/hexane to deliver 56.8 g of BR-05 (57%) as a light-yellow liquid. (TLC: EA:Hex (7:93), R_{f}=0.4; 2 times eluted). ¹H NMR (300 MHz, CDCl₃): δ 6.92 (t, J=7.0 Hz, 2H), 6.86-6.68 (m, 2H), 6.21 (dd, J=15.7, 0.8 Hz, 1H), 5.15-5.04 (m, 1H), 4.05-3.93 (m, 1H), 3.65 (s, 3H), 3.52 (t, J=8.7 Hz, 1H), 2.69-2.48 (m, 6H), 2.34 (t, J=7.6 Hz, 2H), 2.05-1.87 (m, 3H), 1.71-1.55 (m, 2H), 1.43-1.23 (m, 4H), 0.90 (t, J=6.9 Hz, 3H), 0.79 (s, 9H), 0.01 (s, 3H), −0.04 (s, 3H); ¹³C NMR (75 MHz, CDCl₃): δ 200.29, 174.13, 157.33, 145.93, 131.40, 129.27, 128.99, 123.44, 121.96, 120.58, 84.12, 76.50, 58.61, 51.55, 49.39, 42.69, 40.92, 33.63, 31.59, 29.34, 25.68, 24.85, 24.04, 22.59, 17.98, 14.04, −4.57, −4.72.

### Example 7: Synthesis of Compound of Formula BR-19—methyl 4-((1R,2R,3aS,8bS)-2-((tert-butyldimethylsilyl)oxy)-1-((S,E)-4-methyl-3-oxooct-1-en-6-yn-1-yl)-2,3,3a,8b-tetrahydro-1H-cyclopenta[b]benzofuran-5-yl)butanoate

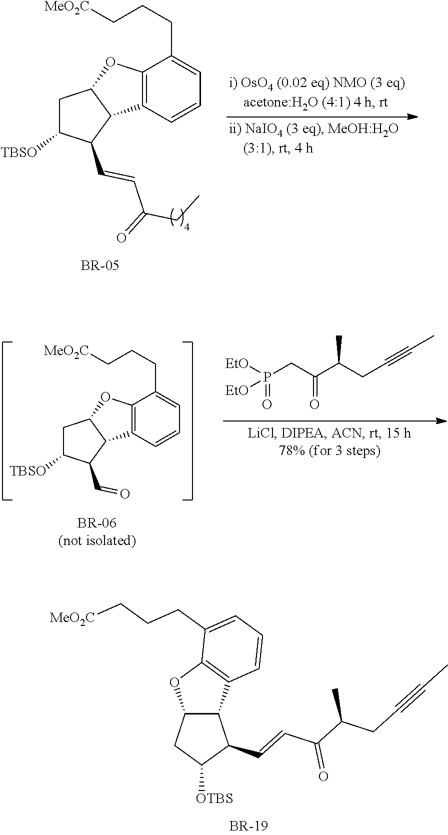

90 g (0.174 mmol, 1.0 eq) of BR-05 was dissolved in acetone (1.44 L, 16 V) and H₂O (0.36 L, 4 V) (4:1) under argon atmosphere at RT and 109 mL (0.524 mol, 3.0 eq) of NMO (50 wt % in water) was added in one portion and protected from light. OsO₄ (0.16 M solution in water, 21.9 mL, 0.0035 mol, 0.02 eq) was added dropwise over 5 mins at RT and stirred at rt for 4 hours under argon atmosphere. TLC showed complete consumption of BR-05 (EA/Hex (1:3)). Solvent (acetone) was removed at reduced pressure to give a thick oil and then 900 mL (10 v/w) of DCM and 900 mL (10 v/w) of H₂O were added thereto, followed by layer separation. The water layer was extracted again with DCM (10 v/w×2). The separated organic layers are combined, dried on Na₂SO₄, filtered followed by solvent evaporation. The crude diol was used in the next step without further purification after drying for 30 mins on high vacuum. Crude diol was dissolved in 1.21 L (13.5 v/w) of MeOH and 0.40 L (4.5 V) of H₂O (3:1). Then, 112.2 g (0.524 mol, 3.0 eq with respect to BR-05) of NaIO₄ was added portion wise over 1 hour (during addition, internal temperature was raised from 25 to 45° C., so added portion wise) and stirred at rt for 4 hours. TLC showed complete consumption of diol (EA/Hex (1:3)). After evaporation of MeOH on rotavapor at 30° C., to the resulting slurry 900 mL (10 v/w) of ethyl acetate and 900 mL (10 v/w) of H₂O were added and stirred at RT for 30 mins. The organic layer was separated, and the aqueous layer was extracted again with ethyl acetate (900 mL, 10 v/w). The separated organic layers are combined, dried on Na₂SO₄, filtered followed by solvent evaporation. The crude aldehyde was used in the next step without further purification after drying for 5 hours on high vacuum (Make sure to eliminate ethyl acetate, crude aldehyde was analyzed by ¹H NMR to check residual solvents). Lithium chloride (14.8 g, 0.35 mol, 2 equivalents with respect to BR-05) was transferred to 5 L 4-neck RB flask under argon, dried on high vacuum for 2 hours (heating RBF with hot gun for 10 mins) and anhydrous acetonitrile (592 mL, 40 V with respect to LiCl)) was added. In another RB flask, 91 g of ω-chain (0.35 mol, 2 equivalents with respect to BR-05) was dissolved in acetonitrile (1.09 L, 12 V with respect to ω-chain) under argon and added N,N-diisopropylethylamine (DIPEA) (54.82 mL, 0.314 mole, 1.8 equivalents with respect to BR-05) dropwise at RT over 5 mins and stirred for 10 to 20 mins. This solution was added using cannula to above prepared LiCl solution at rt over 30 mins (no temp raise). A solution of the crude aldehyde (90 g, BR-06) in acetonitrile (1.08 L, with respect to crude aldehyde) was added dropwise at RT and the mixture was stirred at room temperature for 15 h (Aldehyde and product have same R_{f} on TLC, so reaction was monitored by using ¹H NMR signals of aldehyde). The reaction mixture was cooled to 0˜5° C. added ethyl acetate (900 mL, 10 V) and saturated aqueous ammonium chloride solution (450 mL, 5 V) and the mixture was partitioned by diluting with water (450 mL, 5V). The organic layer was collected, and the aqueous layer was extracted with ethyl acetate (5 V). The combined organic layers were washed with brine (0.9 L, 10 V) and dried on Na₂SO₄, filtered, and concentrated under reduced pressure. The crude product was purified by column chromatography using ethyl acetate/hexane (BR-19, 71 g, 78% yield; light yellow liquid). (TLC: EA:Hex (1:4), R_{f}=0.5). ¹H NMR (300 MHz, CDCl₃): δ 7.00-6.80 (m, 3H), 6.73 (t, J=7.4 Hz, 1H), 6.28 (dd, J=15.5, 0.8 Hz, 1H), 5.15-5.03 (m, 1H), 4.06-3.94 (m, 1H), 3.65 (s, 3H), 3.52 (t, J=8.8 Hz, 1H), 2.97-2.80 (m, 1H), 2.70-2.52 (m, 4H), 2.52-2.39 (m, 1H), 2.39-2.19 (m, 3H), 2.05-1.85 (m, 3H), 1.75 (t, J=2.5 Hz, 3H), 1.20 (d, J=7.0 Hz, 3H), 0.79 (s, 9H), 0.01 (s, 3H), −0.04 (s, 3H); ¹³C NMR (75 MHz, CDCl₃): δ 201.59, 174.11, 157.33, 146.79, 129.83, 129.23, 129.00, 123.46, 121.89, 120.57, 84.11, 77.11, 76.73, 76.48, 58.80, 51.54, 49.50, 44.05, 42.71, 33.62, 29.33, 25.68, 24.84, 22.33, 17.96, 16.50, 3.59, −4.60, −4.74.

### Example 8: Synthesis of Compound of Formula BR-21—methyl 4-((1R,2R,3aS,8bS)-2-hydroxy-1-((3S,4S,E)-3-hydroxy-4-methyloct-1-en-6-yn-1-yl)-2,3,3a,8b-tetrahydro-1H-cyclopenta[b]benzofuran-5-yl)butanoate

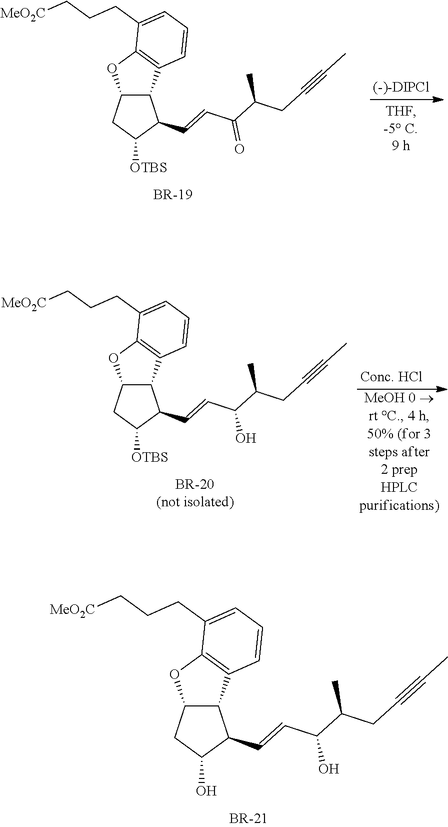

To a solution of BR-19 (70.0 g, 0.133 mol) in anhydrous THF (700 mL, 10 V) was added a solution of (−)-DIPCl (0.53 mol, 314 mL; 1.7 M in hexane) under argon at −10° C. over 45 mins. The reaction was warmed to −5° C. and stirred at this temperature for 17 h. Quenched with 10 V of saturated ammonium chloride solution (700 mL). Warmed to RT and diluted with 700 mL of EA (10 V). After stirring the mixture for 30 min at room temperature, layers were separated, and the aqueous layer was extracted with EtOAc (5 V). The combined organic layers were dried over Na₂SO₄ and concentrated in vacuo after filtration. The crude product was co-distilled with methanol (1 V×3) and used in the next step without further purification after drying for 30 mins on high vacuum. Crude BR-20 was dissolved in 1.14 L (16.24 V) of MeOH and cooled to 0˜5° C. 8.42 mL (0.1199 V) of Conc. HCl was added dropwise over 10 mins (The final concentration of HCl in MeOH is 0.1 M). Warmed to Rt and stirred for 4 hours under argon. TLC showed complete consumption of BR-20 (EA/Hex (1:9, eluted 3 times)). Methanol was evaporated at 30° C. on rotavapor and dried for 10 mins. The crude residue was dissolved in 700 mL (10 V) of ethyl acetate and the EA layer was washed with sat. NaHCO₃ solution (700 mL, 10 V). The organic layer was dried on Na₂SO₄, filtered, and concentrated under reduced pressure. DIP-Cl and its byproducts were removed by filter column chromatography using EA/hexane. Second column purification was performed using EA/Hex (10% gradient) to separate C-15 epimer. Please see Batch No. 1356-109 for more details on column fractions. After 1st (10% IPA/Hex) and 2nd (EA/Hex) prep-HPLC purification 24.33 g (50%) of BR-21 was obtained as white solid. (TLC: EA:Hex (3:1), R_{f}=0.5). ¹H NMR (300 MHz, CDCl₃): δ 6.92 (t, J=7.8 Hz, 2H), 6.74 (t, J=7.4 Hz, 1H), 5.70-5.47 (m, 2H), 5.12-5.00 (m, 1H), 4.00 (t, J=7.4 Hz, 1H), 3.93-3.77 (m, 1H), 3.64 (s, 3H), 3.49 (brs, 1H, OH), 3.38 (t, J=8.9 Hz, 1H), 3.12 (brs, 1H, OH), 2.73-2.54 (m, 3H), 2.41-2.27 (m, 3H), 2.27-2.18 (m, 2H), 2.05-1.83 (m, 3H), 1.83-1.65 (m, 4H), 0.97 (d, J=6.8 Hz, 3H); ¹³C NMR (75 MHz, CDCl₃): δ 174.31, 157.31, 134.20, 133.46, 129.69, 128.96, 123.41, 121.87, 120.55, 84.18, 77.38, 77.32, 76.43, 76.03, 58.98, 51.61, 50.26, 41.32, 38.27, 33.53, 29.29, 24.83, 22.48, 15.85, 3.62.

### Example 9: Synthesis of Compound of Beraprost 314-d Sodium (BPS-314d)—Sodium 4-((1R,2R,3aS,8bS)-2-hydroxy-1-((3S,4S,E)-3-hydroxy-4-methyloct-1-en-6-yn-1-yl)-2,3,3a,8b-tetrahydro-1H-cyclopenta[b]benzofuran-5-yl)butanoate

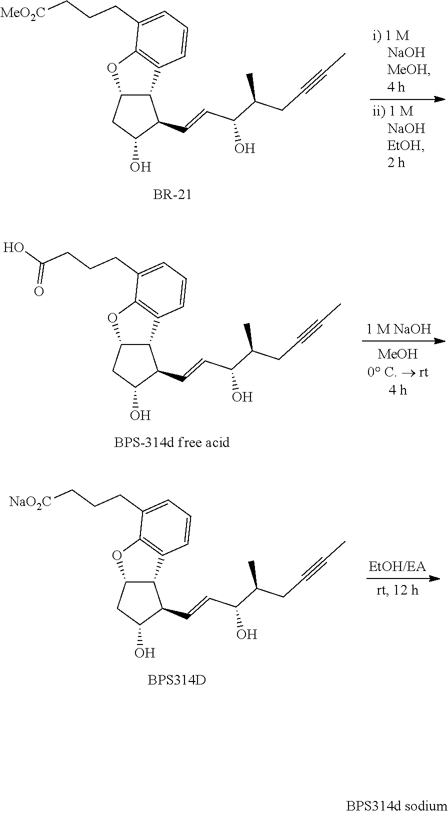

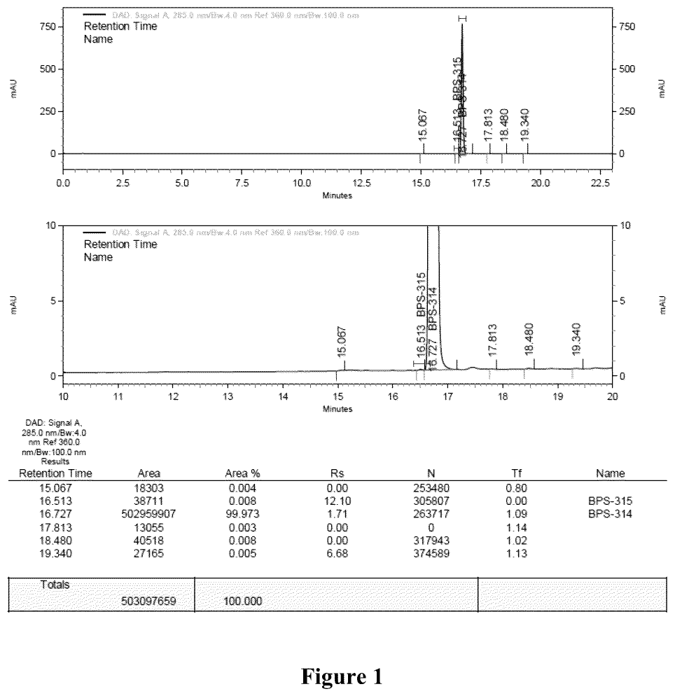

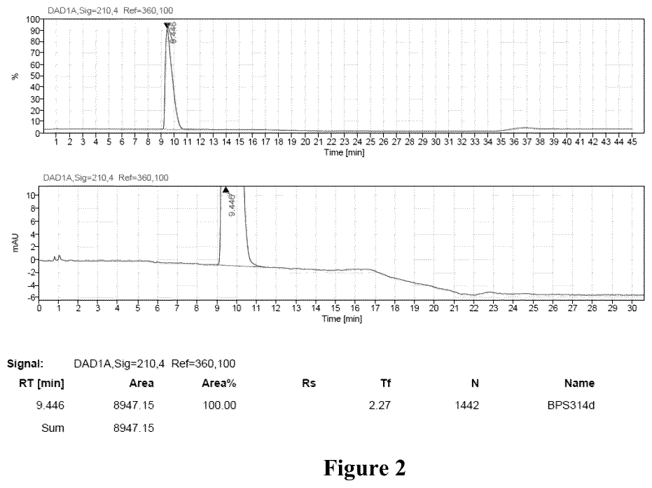

To a solution of BR-21 (24.33 g, 58.98 mmol) in MeOH (121.65 mL, 5 V) was added 1 N NaOH (123.85 mmol, 124 mL) at 0° C. over 30 mins. The reaction mixture was warmed to RT and stirred for 4 h and concentrated in vacuo. After evaporation, resulting residue was dissolved in 240 mL of H₂O (10 V) and the aqueous layer was washed with diethyl ether (10 V×2). The aqueous layer was acidified (pH 2-3) with 1 M HCl solution at 0° C. (precipitate formed). To the resulting precipitate was added 240 mL of ethyl acetate (10 V), warmed to RT and stirred for 30 mins. The separated aqueous layer was extracted with EtOAc (10V×2). The combined organic layers were washed with water (20 V), brine solution (20 V), dried over Na₂SO₄ and concentrated in vacuo after filtration. The BPS-314d free acid was used in the next step without further purification after drying for overnight on high vacuum at 35° C. (24.0 g). (TLC: EA:Hex (3:1), R_{f}=0.2). To a solution of BPS-314d free acid (24.0 g, 60.22 mmol) in EtOH (156 mL, 6.5 V) was added 1 N NaOH (60.82 mmol, 60.8 mL) solution at 0° C. over 30 mins. The reaction mixture was warmed to RT and stirred for 2 h and filtered to remove foreign particles. After solvent evaporation, dried for overnight on high vacuum at 30° C. (25 g). 25 g of BPS-314d sodium salt was dissolved in 75 mL of ethanol (3 V, not dissolved) at RT. To this was added 500 mL of ethyl acetate (20 v) and stirred at RT for overnight. The resulting precipitate was filtered and the filter cake was washed 3 times with 2 V of ethyl acetate. Dried in a vacuum oven at 60° C. for 8 hours (BR-1356-117 and NX-1297-151). Then, using a mortar and pestle, grind the solids to a powder. At 60° C., drying was continued for a further 12 hours and analyzed HPLC. 23.08 g of spec-in BPS-314d sodium was produced from BR-21 in 3 steps with a yield of 98%, a purity of 99.97% (FIG. 1) by the achiral UPLC method, and a purity of 100% by the chiral HPLC method (FIG. 2). ¹H NMR (300 MHz, DMSO-d6): δ 6.90 (t, J=6.7 Hz, 2H), 6.70 (t, J=7.4 Hz, 1H), 5.63 (dd, J=15.4, 7.7 Hz, 1H), 5.45 (dd, J=15.4, 6.9 Hz, 1H), 5.11 (brs, 1H, OH), 5.07-4.96 (m, 1H), 4.91 (brs, 1H, OH), 3.87-3.78 (m, 1H), 3.77-3.65 (m, 1H), 3.38 (t, J=9.0 Hz, 1H), 2.59-2.38 (m, 3H), 2.35-2.21 (m, 1H), 2.16 (q, J=8.5 Hz, 1H), 2.08-1.95 (m, 1H), 1.89 (t, J=7.5 Hz, 2H), 1.75 (t, J=2.4 Hz, 3H), 1.72-1.53 (m, 4H), 0.92 (d, J=6.8 Hz, 3H); ¹³C NMR (75 MHz, DMSO-d6): δ 177.27, 156.82, 133.21, 131.48, 130.16, 128.17, 124.20, 121.04, 119.85, 83.51, 78.34, 76.28, 74.84, 74.10, 58.07, 48.96, 41.92, 38.72, 38.05, 29.63, 26.64, 21.61, 15.54, 3.23.

### Example 10: In Vitro Analysis of CTO1681 Activity in CAR T-Cell Assay

The impact of CTO1681 on the efficacy of CD19-targeting CAR T-cells in vitro as well as its ability to reduce CRS-inducing pro-inflammatory cytokine levels in the presence of CAR T-cells and tumor cells in vitro were explored. The objective of the study was to determine if CTO1681 displayed an anti-CRS phenotype while preserving anti-tumor functions of CAR T-cells. CD3 T cells were sorted from one healthy donor PBMCs, stimulated for a short period of time and transduced with CD19 CAR-T lentivirus (LV) containing CD28 and CD3ζ domains. CD19 CAR T-cells were expanded for 6 days and used in the assay.

CD19-targeting CD3 T-cells were treated with 5 increasing concentrations of CTO1681 (0.36 nM, 1.8 nM, 9 nM, 45 nM, and 225 nM) or vehicle for 30 minutes, prior to, and during co-culture with CD19+ Raji lymphoma target cells. Media alone, vehicle, and positive control (dexamethasone) wells were included. Following initial treatment, CAR T-cells (Effectors) were co-cultured with fluorescently labeled CD19+ Raji tumor cells (Target cells) at 3 different Effector:Target cell ratios (10:1, 5:1 and 1:1) and treated with 5 different concentrations of CTO1681 or vehicle for 24 hours. Target cells (Raji cells) were fluorescently labelled with CPD (eBioscience™ Cell Proliferation Dye eFluor™) prior to co-culture to distinguish the Raji cells from the effector cells and allow for analysis of target viability by flow cytometry. Following incubation, supernatant was collected and levels of pro-inflammatory cytokines IL-6 and TNF-α were measured by multiplex Luminex assay and INF-7 was measured via time-resolved fluorescence resonance energy transfer (TR-FRET) assay. These cytokines were selected for quantification as they are early cytokines released following CAR T-cell infusion in vivo that causes hyperactivation of the immune system resulting in acute systemic inflammation, CRS, multiorgan failure, and possible death. In addition, flow cytometry was used to measure the level of Raji tumor/target cell death following CAR T treatment across CTO1681 conditions to measure impact of CTO1681 with CAR T-cell efficacy. Co-cultures were stained with viability dye 7-AAD for measuring viability.

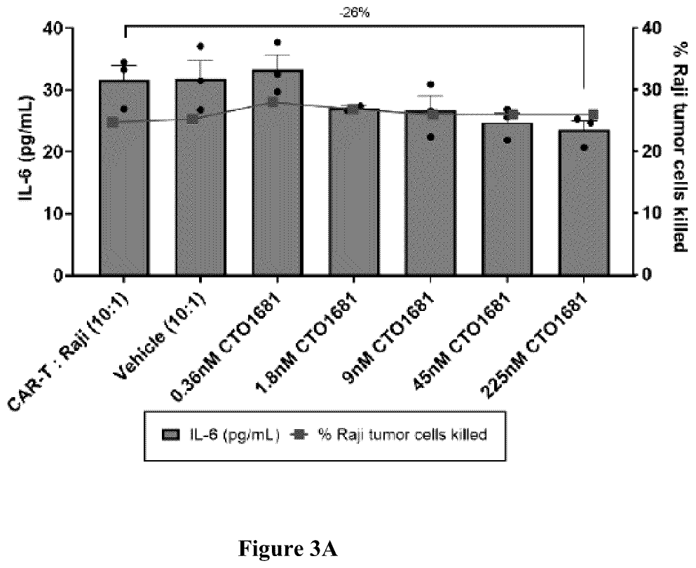

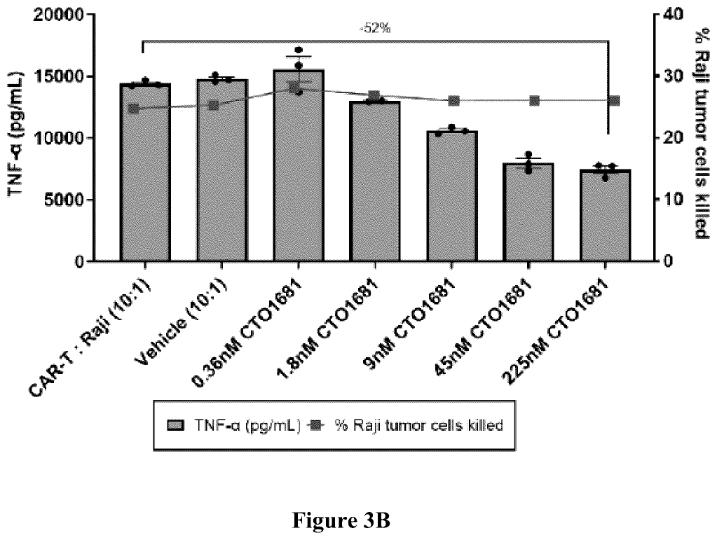

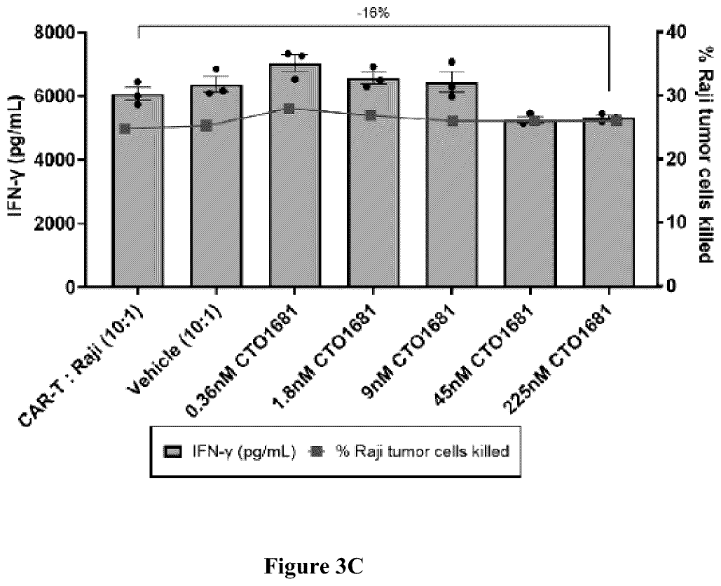

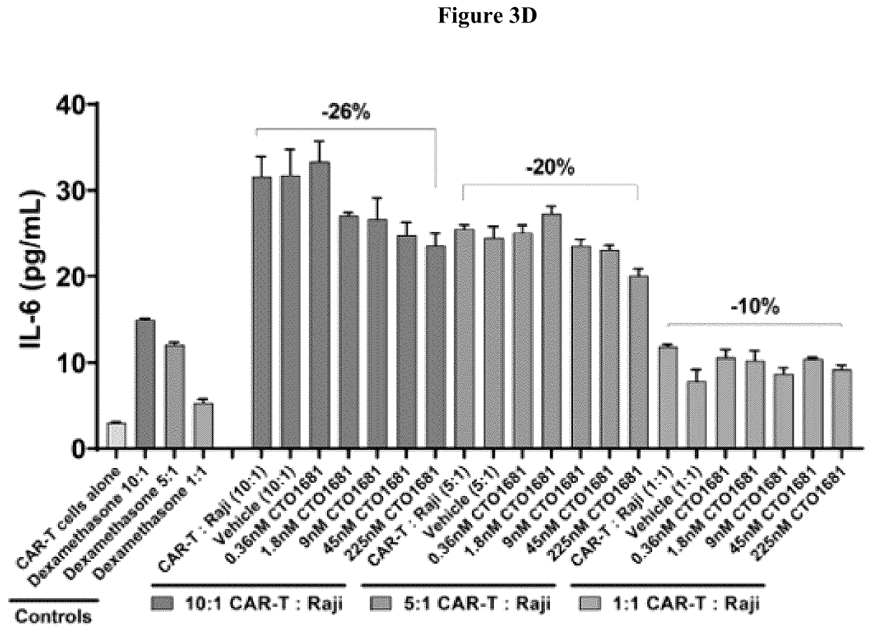

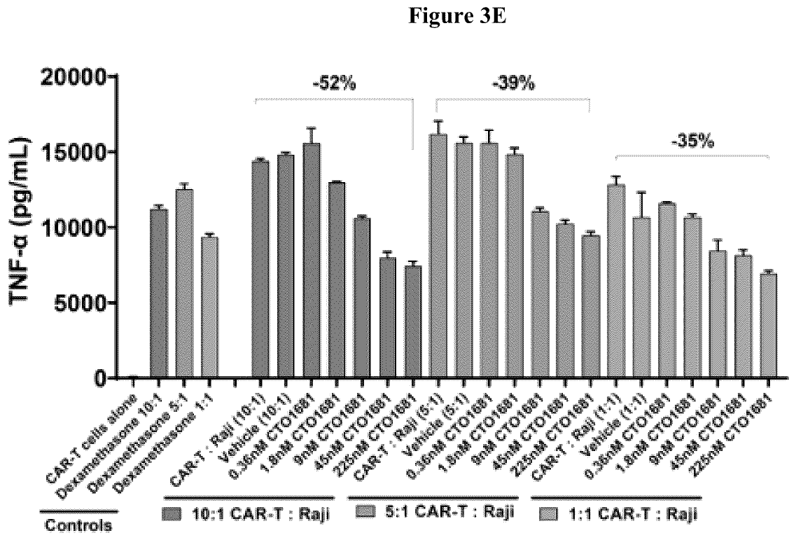

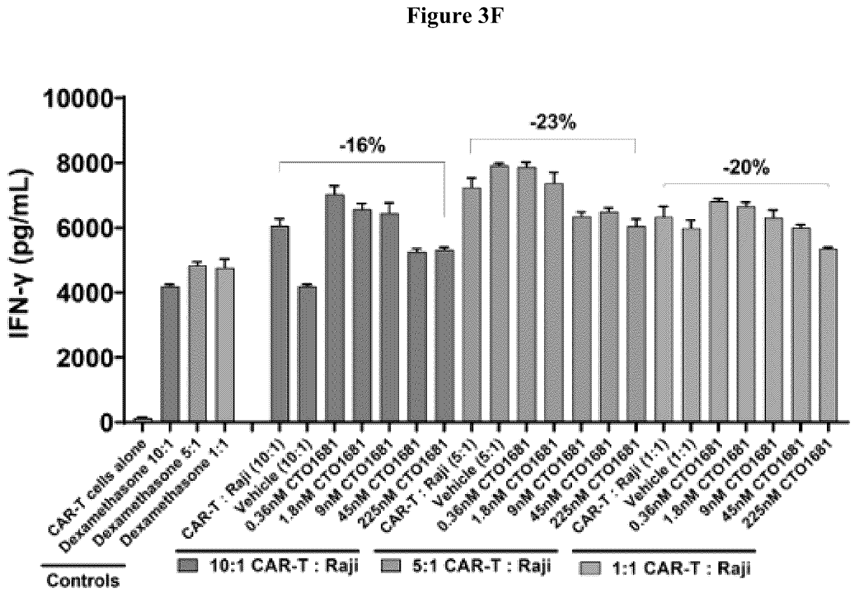

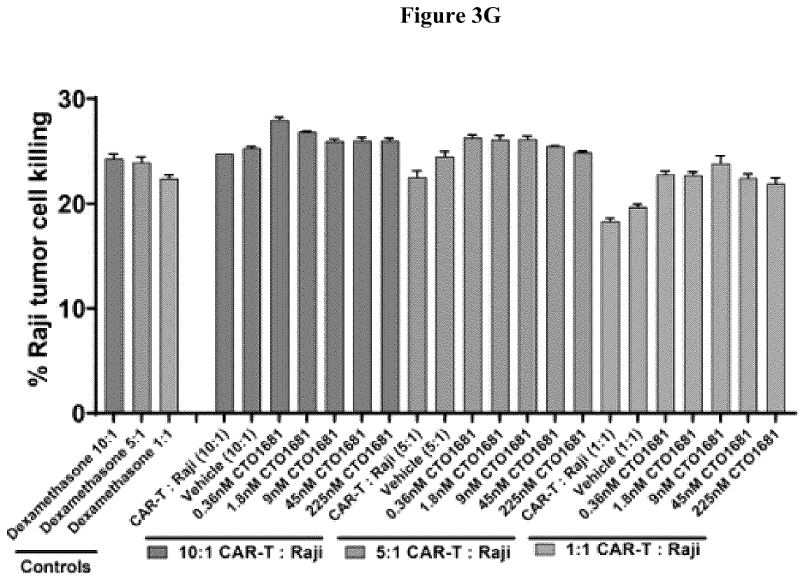

The results for 10:1 ratio of Effectors to Target cells are shown in FIG. 3A, FIG. 3B and FIG. 3C. Maximum reduction reached approximately 50% for TNF-α, 26% for IFN-7, and 23% for IL-6, respectively. The study found that CTO1681 did not impact or interfere with the tumor/target cell killing effect of CD19-directed CAR T-cells in vitro (for example, CTO1681 does not interfere with CAR T-cell efficacy). All concentrations of CTO1681 showed target cell killing. As expected, CTO1681 dose-dependently reduced pro-inflammatory and CRS-inducing cytokine levels IL-6, TNF-α, and INF-γ. Similar results were obtained at 5:1 and 1:1 ratios of Effectors to Target cells (FIG. 3D, FIG. 3E, FIG. 3F and FIG. 3G). Over a dozen cytokines have been shown to be elevated following CAR T-cell infusion in patients and CTO1681 has been shown to reduce the level of each cytokine in non-CAR T models. The results here demonstrating CTO1681 is able to significantly reduce the levels of IL-6, TNF-α, and IFN-γ aligns with previous findings that CTO1681 can reduce each of these cytokine levels in infectious disease models in vitro and in vivo. The results also provide additional validation that the CTO1681 targets an underlying source of pro-inflammatory cytokine release from immune cells. Therefore, CTO1681 is an optimal therapeutic candidate for CAR T-cell therapies. Collectively, results from this study demonstrate in vitro that CTO1681 does not interfere with CAR T-cell efficacy. It also provides strong evidence that CTO1681 acts on CAR T-cells to significantly reduce the release of pro-inflammatory cytokines critical in initiating a hyperactive immune response and life-threatening conditions including CRS.

### Example 11: In Vivo Effects of CTO1681 in CAR T-Cell Efficacy in NSG Tumor Bearing Mice

The effects if any, of CTO1681 on CAR T-cell activity in vivo were measured. Human T-cells expressing CD19-CD28-CD3Z CAR construct was used in this experiment.

6-7 week old NSG™ (NOD SCID gamma) mice were engrafted with 1×10⁶ luciferase (luc) expressing Raji tumor cells (herein referred to as “Raji-luc” tumor cells) each on Day 1 to allow for tumor growth over a 7-day period prior to the commencement of the treatment. CAR T-cell infusion was performed intravenously via lateral tail vein on (5×10⁶ CD19 CAR T-cells generated from healthy donors). NSG mice are severely immunodeficient, which facilitates ready tumor engraftment and subsequent clearance due to CAR T-cells can be measured.

Tumor growth after engraftment and subsequent clearance were monitored once weekly (starting on Day 7) using IVIS imaging to detect luciferase signal in living mice. CTO1681 administration began on Day 8, two days before CAR T-cell infusion, with injections twice a day for 14 days. Mice received total daily CTO1681 doses of 0.3 mg/kg/d, 0.2 mg/kg/d, or 0.15 mg/kg/d via two injections (BID). CTO1681 doses were selected based on the pharmacokinetic profile of the drug in mice. Control groups included mice receiving Raji-luc tumor cells without CAR T-cell treatment, mice receiving Raji-luc tumor cells and a CAR T-cell infusion, and mice receiving Raji-luc tumor cells with vehicle treatments. Mice were monitored for body temperature and health evaluations (weight, movement, posture, fur condition, etc.) throughout the entire study.

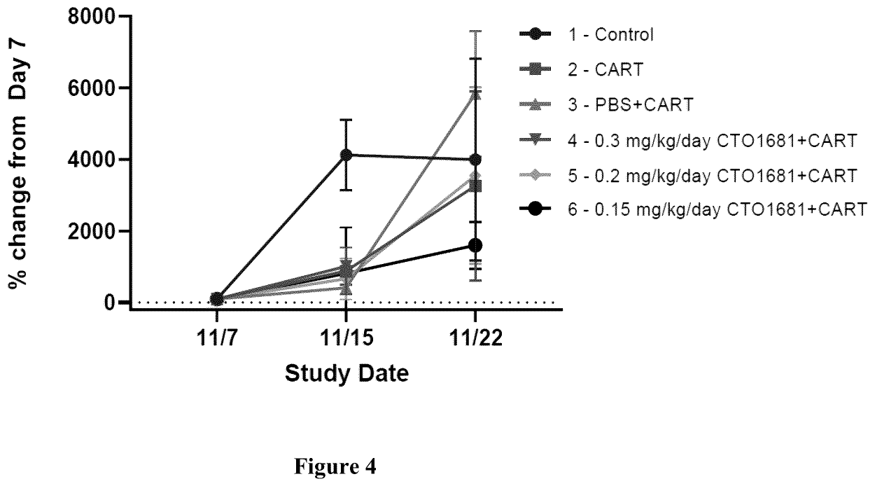

Tumor burden was confirmed prior to treatment commencement, with CTO1681 treatment groups starting BID dosing 48 hours prior to CAR T-cell administration. 2 way ANOVA for day 15 measurements found statistical difference (p=<0.0001) between Group 1 (no CAR-T treatment) and all other Groups. CD19 CAR-T cells significantly inhibited Raji tumor cell growth. All animals dosed with CD19 CAR-T cells including those that received a co-administration of CTO1681 had a significantly reduced tumor burden of between 3.9- and 9.5-fold increase. Hence treatment with CTO1681 did not affect CD19 CAR T-cell activity at any dose tested. No statistical difference (p=0.17 to 0.99) was found between Groups dosed with CAR-T cells alone (Groups 2 and 3) and animals dosed with both CAR-T cells and CTO1681 at any dose level. IVIS images and luciferin signal quantification are shown at Day 7 and Day 15 after allowing for suitable tumor growth post-engraftment, where Day 7 values represent pre-treatment baseline (FIG. 4). By Day 6 of CAR T-cell treatment (Day 15 of study), tumor burden was remarkably attenuated with no interference in CAR T-cell therapy associated reductions at all three CTO1681 dosages (FIG. 4). Follow-up observations at later timepoints showed disease progression in all experimental groups including those without CTO1681 treatment, indicating renewed replication and growth of Raji-luc cells not eliminated by CAR-T treatment, a phenomenon that is occasionally seen in the NSG tumor model. These data clearly show that CTO1681 administration together with CAR T-cell therapy does not interfere with treatment efficacy in vivo. Additionally, CTO1681 did not cause any increase in clinical morbidities, including weight loss or limb movement. In conclusion, CAR T-cell treatment with or without CTO1681 caused a drastic reduction in tumor burden as compared to mice who did not receive any treatment.

1. A method of preparing a product comprising a compound of Formula I having the structure: 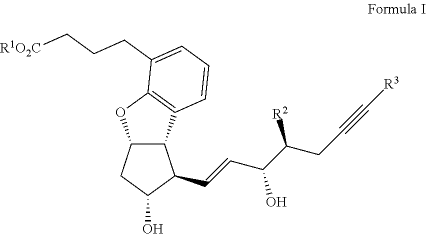
wherein
R¹ is selected from H and a cation;
R² and R³ are independently C₁₋₆ alkyl;
the method comprising the steps of:
(a) performing a condensation reaction on the compound 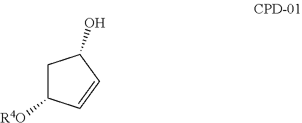
to form the compound 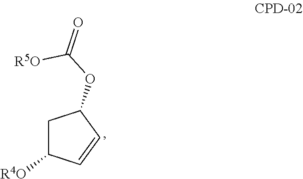
wherein R⁴ is C(O)C₁₋₆ alkyl and R⁵ is C₁₋₆ alkyl;
(b) coupling the carbonate of CPD-02 with 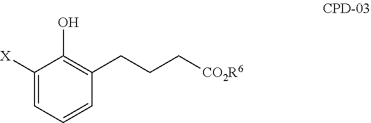
to form the compound 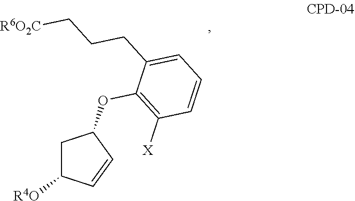
wherein is R⁶ is C₁₋₆ alkyl and X is halide;
(c) hydrolyzing CPD-04 to form the compound 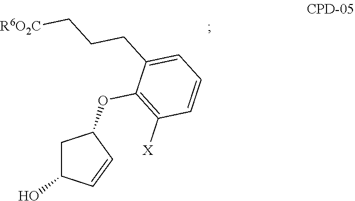
(d) protecting the alcohol of CPD-05 to form the compound 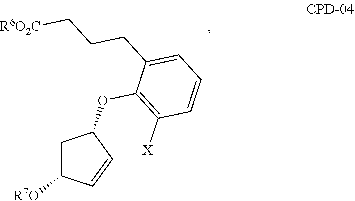
wherein R⁷ is a hydroxy protecting group;
(e) performing a radical cyclization and trapping reaction on CPD-06 with 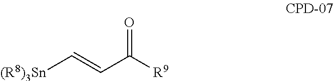
to form the compound 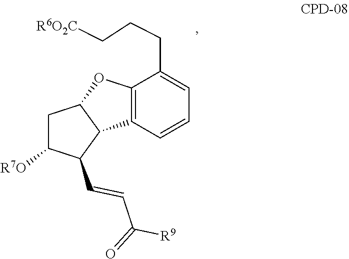
wherein R⁸ and R⁹ are independently C₁₋₆ alkyl; and
(f) converting CPD-08 to a compound of Formula I;
wherein the compound of Formula I is at least about 90% pure;
wherein the total amount of the impurities is in an amount of less than about 10.0%; and
wherein any individual impurity is present in an amount of less than about 1.0%.

2. The method of claim 1, further comprising cleaving the double bond of CPD-08 to from the compound

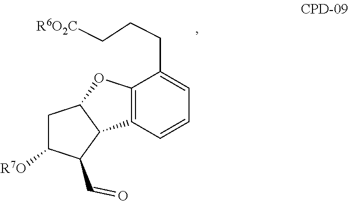

3. The method of claim 2, further comprising coupling CPD-09 with 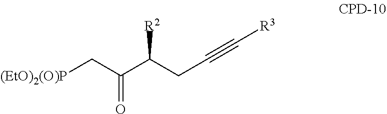
to form the compound

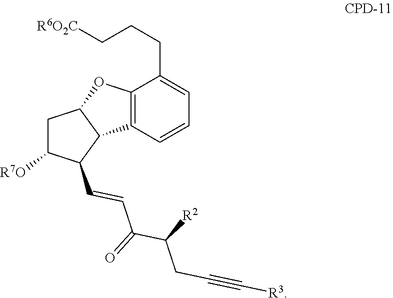

4. The method of claim 3, further comprising reduction of the ketone of CPD-11 to form the compound

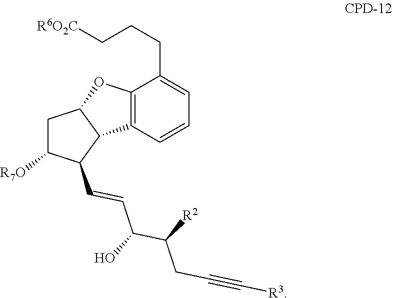

5. The method of claim 4, further comprising the deprotection of CPD-12 to form the compound

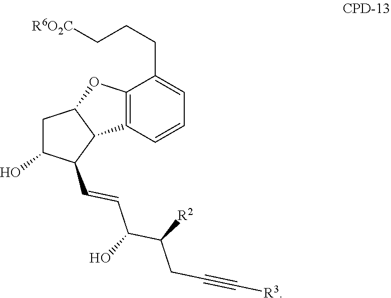

6. The method of claim 5, further comprising hydrolyzing CPD-12 to form the compound Formula I.

7. The method of claim 1, wherein R¹ is Na⁺.

8. The method of claim 1, wherein R², R³, and R⁶ are methyl.

9. The method of claim 1, wherein R⁴ is C(O)Me.

10. The method of claim 1, wherein R⁵ is ethyl.

11. The method of claim 1, wherein R⁷ is tert-butyldimethylsilyl.

12. The method of claim 1, wherein R⁸ is butyl.

13. The method of claim 1, wherein R⁹ is pentyl.

14. The method of claim 1, wherein the compound of Formula I is:

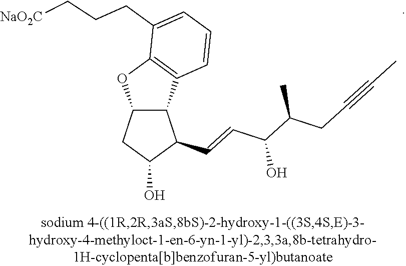

15. The method of claim 1, wherein the method does not produce more than about 1.0% of an isomer other than the compound of Formula I.

16. The method of claim 1, wherein the method does not produce more than about 0.15% of an isomer other than the compound of Formula I.

17. The method of claim 1, wherein the method does not produce more than about 0.1% of an isomer other than the compound of Formula I.

18. The method of claim 1, wherein the method does not produce more than about 0.05% of an isomer other than the compound of Formula I.

19. The method of claim 1, wherein the compound of Formula I is at least about 95% pure.

20. The method of claim 1, wherein the compound of Formula I is at least about 99% pure.

21. The method of claim 1, wherein the compound of Formula I is at least about 99.8% pure.

22. The method of claim 1, wherein any individual impurity is present in an amount of less than about 0.15%.

23. The method of claim 1, wherein any individual impurity is present in an amount of less than about 0.1%.

24. The method of claim 1, wherein any individual impurity is present in an amount of less than about 0.05%.

25. The method of claim 1, wherein the compound of Formula I is prepared in an overall yield of at least about 5%.

26. The method of claim 1, wherein the compound of Formula I is prepared in an overall yield of at least about 10%.

27. The method of claim 1, wherein the total amount of the impurities is in an amount of less than about 5.0%.

28. The method of claim 1, wherein the total amount of the impurities is in an amount of less than about 1.0%.

29. The method of claim 1, wherein the total amount of the impurities is in an amount of less than about 0.2%.

30. The method of claim 1, wherein the impurity is selected from: 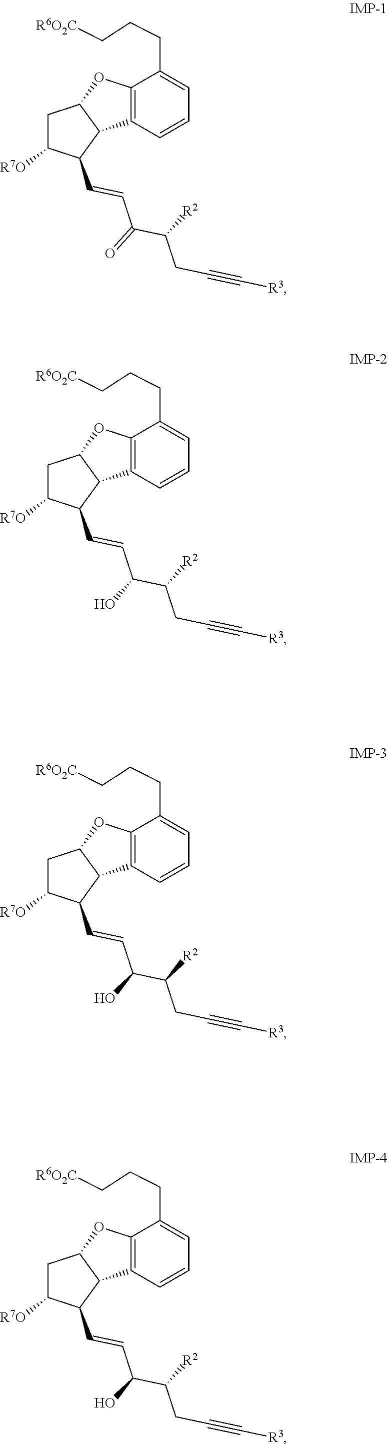 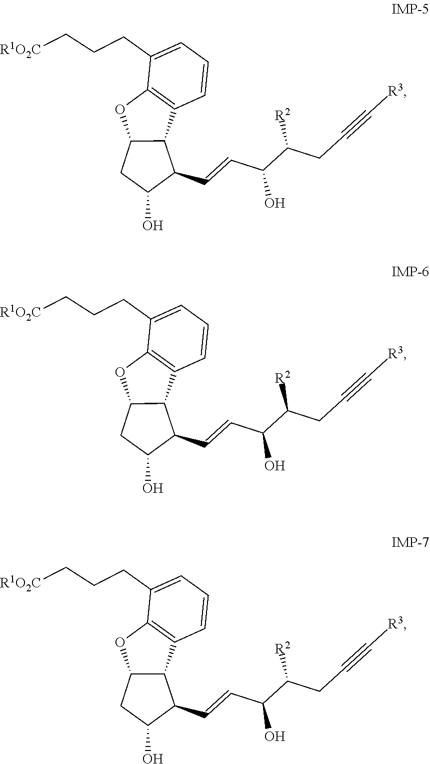
and a combination thereof.

31. The method of claim 1, wherein the impurity is selected from: 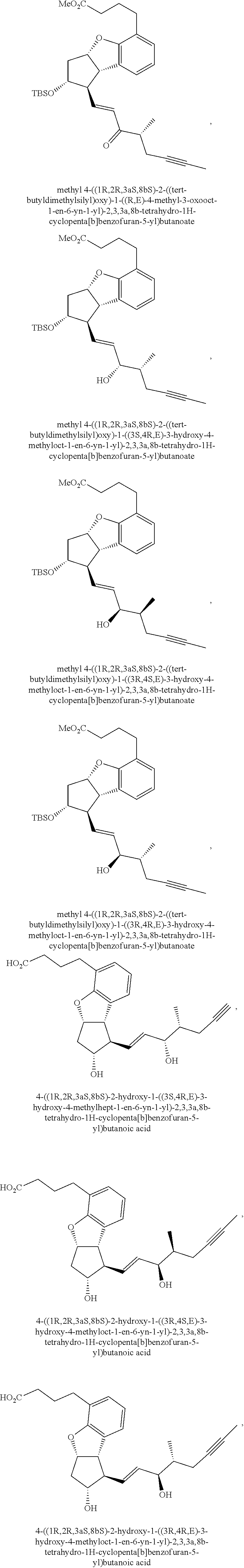 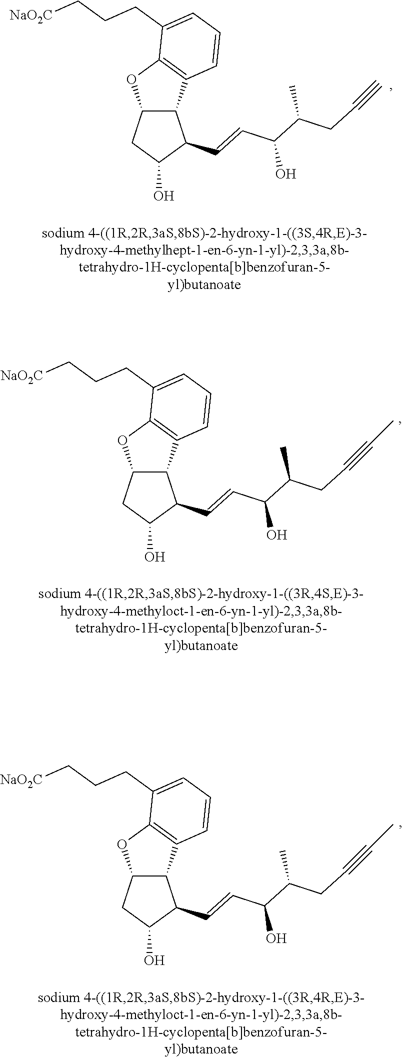
and a combination thereof.

32. The method of claim 1, wherein, Formula I is prepared without the use of chiral chromatography.

33. The compound of claim 14, wherein, sodium 4-((1R,2R,3aS,8bS)-2-hydroxy-1-((3S,4S,E)-3-hydroxy-4-methyloct-1-en-6-yn-1-yl)-2,3,3a,8b-tetrahydro-1H-cyclopenta[b]benzofuran-5-yl)butanoate is prepared without the use of chiral chromatography.

34. A compound of the formula: 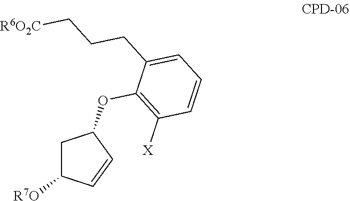
wherein
R⁶ is C₁₋₆ alkyl;
R⁷ is a hydroxy protecting group; and
X is halide.

35. The compound of claim 32, wherein the compound is:

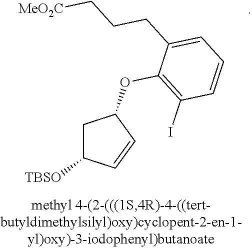 [SMILES: COC(=O)CCCc1cccc(I)c1O[C@@H]1C=C[C@H](O[Si](C)(C)C(C)(C)C)C1]

36.-37. (canceled)

38. A method of treating cytokine release syndrome (CRS) in a subject, the method comprising administering to the subject a composition comprising an effective amount of the compound of Formula I or a pharmaceutically acceptable salt thereof according to claim 1; wherein the CRS is treated.

39. (canceled)
